SEQUENCE LISTING (28 sequences)

SEQ ID NO: 1 - protein, 449 aa, Artificial Sequence
  Heavy Chain A for trastuzumab (HER_CH-_T350V_L351Y_F405A_Y407V_no_C-term_Lys)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 2 - protein, 107 aa, Artificial Sequence [sequence omitted]

SEQ ID NO: 3 - protein, 439 aa, Artificial Sequence
  Heavy Chain B with HSAdIII (HSAdIII-HET_Fc_001b_no_C-term_Lys)
GVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 4 - protein, 240 aa, Artificial Sequence
  Heavy Chain B with Lys6 (Lys6-HET_Fc_001b_no_C-term_Lys)
GKKKKKKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 5 - protein, 228 aa, Artificial Sequence
  Heavy Chain B with Gly-Cys (Fc_CH-B_N-term_GlyCys_T350V_T366L_K392L_T394W_no_C-term_Lys)
GCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 6 - protein, 227 aa, Artificial Sequence
  Heavy Chain B with Cys (Fc_CH-B_N-term_Cys__T350V_T366L_K392L_T394W_no_C-term_Lys)
CDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 7 - protein, 232 aa, Artificial Sequence
  Heavy chain B - Fc region
GEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 8 - protein, 450 aa, Artificial Sequence
  Polypeptide Heavy Chain (Variant 506)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - skipped
  Polypeptide Light Chain (Variant 506)

SEQ ID NO: 10 - protein, 585 aa, Artificial Sequence [sequence omitted]

SEQ ID NO: 11 - DNA, 1755 bp, Artificial Sequence
  Exemplary DNA Sequence Human Serum Albumin (HSA)
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 12 - protein, 449 aa, Artificial Sequence; identical to SEQ ID NO: 1
  Polypeptide Heavy Chain A (Variant 5110)

SEQ ID NO: 13 - DNA, 1347 bp, Artificial Sequence
  DNA Heavy chain A (Variant 5110)
gaagtccagctggtcgaaagcggaggaggactggtgcagccaggagggtctctgcgactgagttgcgccgcttcaggcttcaacatcaaggacacctacattcactgggtgcgccaggctcctggaaaaggcctggagtgggtggcacgaatctatccaactaatggatacacccggtatgcagacagcgtgaagggccggttcaccattagcgcagatacatccaaaaacactgcctacctgcagatgaacagcctgcgagccgaagatactgctgtgtactattgcagtcggtggggaggcgacggcttctacgctatggattattgggggcagggaaccctggtcacagtgagctccgcatctacaaaggggcctagtgtgtttccactggccccctctagtaaatccacctctgggggaacagcagccctgggatgtctggtgaaggactatttcccagagcccgtcactgtgagttggaactcaggcgccctgacatccggggtccatacttttcctgctgtgctgcagtcaagcggcctgtactctctgtcctctgtggtcaccgtgccaagttcaagcctggggactcagacctatatctgcaacgtgaatcacaagccaagcaatacaaaagtcgacaagaaagtggaacccaagagctgtgataaaacacatacttgccccccttgtcctgcaccagagctgctgggaggaccatccgtgttcctgtttccacccaagcctaaagacaccctgatgatttccaggactccagaagtcacctgcgtggtcgtggacgtgtctcacgaggaccccgaagtcaagttcaactggtacgtggatggcgtcgaggtgcataatgccaagacaaaacccagggaggaacagtacaactcaacttatcgcgtcgtgagcgtcctgaccgtgctgcaccaggactggctgaacggcaaagagtataagtgcaaagtgagcaataaggctctgcccgcacctatcgagaaaaccattagcaaggccaaagggcagcctagagaaccacaggtctacgtgtatcctccaagcagggacgagctgaccaagaaccaggtctccctgacatgtctggtgaaagggttttaccccagtgatatcgctgtggagtgggaatcaaatggacagcctgaaaacaattataagaccacaccccctgtgctggacagcgatggcagcttcgctctggtctccaagctgactgtggataaatctcggtggcagcagggcaacgtctttagttgttcagtgatgcatgaggcactgcacaatcattacacccagaagagcctgtccctgtctcccggc

SEQ ID NO: 14 - protein, 438 aa, Artificial Sequence
  Polypeptide Heavy Chain B (Variant 5110)
VEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 15 - DNA, 1314 bp, Artificial Sequence
  DNA Heavy Chain B (Variant 5110)
gtggaagaacctcagaacctgatcaaacagaattgcgagctgttcgaacagctgggcgaatacaagtttcagaacgccctgctggtgcggtataccaagaaagtcccacaggtgagtacccccacactggtcgaggtgtcaagaaatctgggaaaagtgggcagcaaatgctgtaagcaccctgaggctaagaggatgccatgcgcagaagactacctgtctgtggtcctgaaccagctgtgtgtgctgcatgaaaaaactcccgtcagcgatcgcgtgaccaagtgctgtacagagagtctggtgaaccggagaccatgcttctcagccctggaggtcgacgaaacctatgtgcccaaagagtttaatgccgaaactttcacctttcacgctgatatctgtaccctgtctgagaaggaacgacagattaagaaacagacagctctggtcgagctggtgaagcataaaccaaaggcaaccaaagaacagctgaaggccgtgatggacgatttcgccgcttttgtggagaaatgctgtaaggctgacgataaggaaacatgcttcgcagaggaagggaagaaactggtggcagcaagccaggctgcactgggactggccgctgaacccaaaagctccgacaagacacacacttgcccaccttgtccagcaccagagctgctgggaggaccatccgtgttcctgtttccacccaaacctaaggatacactgatgatctctaggactcccgaggtcacctgtgtggtcgtggacgtcagtcacgaggaccccgaagtcaagtttaactggtacgtggacggcgtcgaggtgcataatgccaaaactaagcctagggaggaacagtacaattcaacatatcgcgtcgtgagcgtcctgactgtgctgcatcaggattggctgaacgggaaggagtataaatgcaaggtgagcaacaaggcactgcctgccccaatcgagaagacaatttccaaagcaaagggacagccccgagaacctcaggtctacgtgctgcctccaagccgggacgagctgactaaaaaccaggtctccctgctgtgtctggtgaagggcttctatccttccgatattgctgtggagtgggaatctaatgggcagccagagaacaattacctgacttggccccctgtgctggactctgatggaagtttctttctgtattccaaactgaccgtggacaagtctcggtggcagcagggcaacgtcttttcttgtagtgtgatgcacgaggccctgcacaatcattacacacagaagtcactgagcctgtcccccggg

SEQ ID NO: 16 - protein, 214 aa, Artificial Sequence
  Polypeptide Light Chain (Variant 5110)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 642 bp, Artificial Sequence
  DNA LIght Chain (Variant 6265)
gatattcagatgacccagtcccctagctccctgtccgcttctgtgggcgacagggtcactatcacctgccgcgcatctcaggatgtgaacaccgcagtcgcctggtaccagcagaagcctgggaaagctccaaagctgctgatctacagtgcatcattcctgtattcaggagtgcccagccggtttagcggcagcagatctggcaccgacttcacactgactatctctagtctgcagcctgaggattttgccacatactattgccagcagcactataccacaccccctactttcggccaggggaccaaagtggagatcaagcgaactgtggccgctccaagtgtcttcatttttccacccagcgacgaacagctgaaatccggcacagcttctgtggtctgtctgctgaacaacttctaccccagagaggccaaagtgcagtggaaggtcgataacgctctgcagagtggcaacagccaggagagcgtgacagaacaggactccaaagattctacttatagtctgtcaagcaccctgacactgagcaaggcagactacgaaaagcataaagtgtatgcctgtgaggtgacccatcaggggctgtcttctcccgtgaccaagtctttcaaccgaggcgaatgt

SEQ ID NO: 18 - protein, 450 aa, Artificial Sequence
  Polypeptide Heavy Chain A (Variant 1040)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 19 - DNA, 1350 bp, Artificial Sequence
  DNA Heavy Chain A (Variant 1040)
gaagtccagctggtcgaaagcggaggaggactggtgcagccaggagggtctctgcgactgagttgcgccgcttcaggcttcaacatcaaggacacctacattcactgggtgcgccaggctcctggaaaaggcctggagtgggtggcacgaatctatccaactaatggatacacccggtatgcagacagcgtgaagggccggttcaccattagcgcagatacatccaaaaacactgcctacctgcagatgaacagcctgcgagccgaagatactgctgtgtactattgcagtcggtggggaggcgacggcttctacgctatggattattgggggcagggaaccctggtcacagtgagctccgcatctacaaaggggcctagtgtgtttccactggccccctctagtaaatccacctctgggggaacagcagccctgggatgtctggtgaaggactatttcccagagcccgtcactgtgagttggaactcaggcgccctgacatccggggtccatacttttcctgctgtgctgcagtcaagcggcctgtactctctgtcctctgtggtcaccgtgccaagttcaagcctggggactcagacctatatctgcaacgtgaatcacaagccaagcaatacaaaagtcgacaagaaagtggaacccaagagctgtgataaaacacatacttgccccccttgtcctgcaccagagctgctgggaggaccatccgtgttcctgtttccacccaagcctaaagacaccctgatgatttccaggactccagaagtcacctgcgtggtcgtggacgtgtctcacgaggaccccgaagtcaagttcaactggtacgtggatggcgtcgaggtgcataatgccaagacaaaacccagggaggaacagtacaactcaacttatcgcgtcgtgagcgtcctgaccgtgctgcaccaggactggctgaacggcaaggagtataagtgcaaagtgagcaataaggctctgcccgcacctatcgagaaaaccattagcaaggccaaagggcagcctagagaaccacaggtctacgtgtatcctccaagcagggacgagctgaccaagaaccaggtctccctgacatgtctggtgaaagggttttaccccagtgatatcgctgtggagtgggaatcaaatggacagcctgaaaacaattataagaccacaccccctgtgctggacagcgatggcagcttcgctctggtctccaagctgactgtggataaatctcggtggcagcagggcaacgtctttagttgttcagtgatgcatgaggcactgcacaatcattacacccagaagagcctgtccctgtctcccggcaaa

SEQ ID NO: 20 - protein, 232 aa, Artificial Sequence
  Polypeptide Heavy Chain B (Variant 1040)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 21 - DNA, 696 bp, Artificial Sequence
  DNA Heavy Chain B (Variant 1040)
gaacctaaaagcagcgacaagacccacacatgccccccttgtccagctccagaactgctgggaggaccaagcgtgttcctgtttccacccaagcccaaagatacactgatgatcagccgaactcccgaggtcacctgcgtggtcgtggacgtgtcccacgaggaccccgaagtcaagttcaactggtacgtggacggcgtcgaagtgcataatgcaaagactaaaccacgggaggaacagtacaactctacatatagagtcgtgagtgtcctgactgtgctgcatcaggattggctgaacggcaaagagtataagtgcaaagtgtctaataaggccctgcctgctccaatcgagaaaactattagtaaggcaaaagggcagcccagggaacctcaggtctacgtgctgcctccaagtcgcgacgagctgaccaagaaccaggtctcactgctgtgtctggtgaaaggattctatccttccgatattgccgtggagtgggaatctaatggccagccagagaacaattacctgacctggccccctgtgctggacagcgatgggtccttctttctgtattcaaagctgacagtggacaaaagcagatggcagcagggaaacgtctttagctgttccgtgatgcacgaagccctgcacaatcattacacccagaagtctctgagtctgtcacctggcaaa

SEQ ID NO: 22 - protein, 227 aa, Artificial Sequence; identical to SEQ ID NO: 6
  Polypeptide Heavy Chain B (Variant 6265)

SEQ ID NO: 23 - DNA, 681 bp, Artificial Sequence
  DNA Heavy Chain B (Variant 6265)
tgcgataaaacacacacttgccccccttgtccagctcccgaactgctgggcgggccaagtgtgttcctgtttccacccaagcccaaagacaccctgatgatcagccgaacccccgaggtcacatgcgtggtcgtggacgtgtcccacgaggaccccgaagtcaagttcaactggtacgtggatggcgtcgaagtgcataatgcaaagacaaaaccacgggaggaacagtacaactctacttatagagtcgtgagtgtcctgaccgtgctgcaccaggactggctgaacggcaaagagtataagtgcaaagtgtctaataaggccctgcctgctccaatcgagaaaaccattagcaaggcaaaagggcagcccagggaacctcaggtctacgtgctgcctccaagtcgcgacgagctgacaaagaaccaggtctcactgctgtgtctggtgaaaggattctatccttccgatattgccgtggagtgggaatctaatggccagccagagaacaattacctgacatggccccctgtgctggacagcgatgggtccttctttctgtattcaaagctgactgtggataaaagcagatggcagcagggaaacgtctttagctgttccgtgatgcatgaagccctgcacaatcattacacccagaagtctctgagtctgtcacctggc

SEQ ID NO: 24 - protein, 207 aa, Artificial Sequence
  Polypeptide (Variant 9992)
EFVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 25 - DNA, 621 bp, Artificial Sequence
  DNA (Variant 9992)
gagttcgtggaggagccccagaacctgatcaagcagaactgcgagctgttcgagcagctgggcgagtacaagttccagaacgccctgctggtgagatacaccaagaaggtgccccaggtgagcacccccaccctggtggaggtgagcagaaacctgggcaaggtgggcagcaagtgctgcaagcaccccgaggccaagagaatgccctgcgccgaggactacctgagcgtggtgctgaaccagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctg

SEQ ID NO: 26 - DNA, 1350 bp, Artificial Sequence
  DNA Heavy Chain (Variant 506)
gaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacactgccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaa

SEQ ID NO: 27 - DNA, 642 bp, Artificial Sequence
  DNA Light Chain (Variant 506)
gacatccagatgacccagtctccatcctccctgtctgcatctgtaggagacagagtcaccatcacttgccgggcaagtcaggacgttaacaccgctgtagcttggtatcagcagaaaccagggaaagcccctaagctcctgatctattctgcatcctttttgtacagtggggtcccatcaaggttcagtggcagtcgatctgggacagatttcactctcaccatcagcagtctgcaacctgaagattttgcaacttactactgtcaacagcattacactaccccacccactttcggccaagggaccaaagtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaagagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 28 - protein, 217 aa, Artificial Sequence
  Human IgG1 Fc sequence 231-447
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2016/0114057 A1
(43) Pub. Date: Apr. 28, 2016

(54) MODULAR PROTEIN DRUG CONJUGATE THERAPEUTIC

(71) Applicant: ZYMEWORKS INC., Vancouver (CA)

(72) Inventors: Surjit Bhimarao DIXIT, Richmond (CA); Gordon Yiu KONG, Vancouver (CA); Thomas C. BOONE, Newbury Park, CA (US); Michael Joseph GRESSER, Ojai, CA (US)

(73) Assignee: ZYEWORKS INC., Vancouver (CA)

(21) Appl. No.: 14/893,706

(22) PCT Filed: May 23, 2014
(86) PCT No.: PCT/CA2014/050486

§ 371 Date: Nov. 24, 2015

Related U.S. Application Data
(60) Provisional application No. 61/827,463, filed on May 24, 2013.

(51) Int. Cl. A61K 47/48; C07K 16/40

(52) U.S. Cl. CPC A61K 47/48646; A61K 47/48384; C07K 16/40; C07K 2317/55; C07K 2317/622; C07K 2317/76

(57) ABSTRACT

The invention provides modular antibody-therapeutic agent conjugates and antibody-detectable-agent conjugates, and methods of using said conjugates in therapeutic and diagnostic procedures.

### CROSS-REFERENCE TO RELATED APPLICATIONS

This application is a non-provisional filing of U.S. Provisional Patent Application No. 61/827,463, filed on May 24, 2013, the disclosure of which is incorporated herein by reference in its entirety for all purposes.

### BACKGROUND OF THE INVENTION

Over a million new cases of cancer will be diagnosed, and over half a million Americans will die from cancer this year. Although surgery can provide definitive treatment of cancer in its early stages, the eradication of metastases is crucial to the cure of more advanced disease. Chemotherapeutic drugs used in combinations provide the standard treatment for metastases and advanced disease. However, the side effects of these treatments seriously diminish the quality of life for cancer patients, and progressions and relapses following surgery and chemotherapy/radiation are common. Thus, despite the expenditure of large amounts of public and private resources over many years, better treatments for cancer are still sorely needed.

Most pharmaceuticals available for cancer therapy are small molecules which traverse cell membranes and become widely distributed through the body. Unfortunately, the systemic use of such conventional antineoplastic drugs is associated with undesirable side effects arising from the lack of specificity, and hence the concomitant toxicity to normal cells. Naturally, the lack of specificity and toxic side effects limit the doses tolerated by a patient for treatment of the disease.

Thus, developing the technology to target therapeutic drugs to cancer cells while sparing normal cells, is the obvious goal for improved treatment of cancer. Macromolecules such as monoclonal antibodies and their derivatives or fragments, which bind to highly expressed tumor antigens and not significantly to normal cells, are the best candidates for targeted therapies.

To date, antibodies have proved to be effective therapeutics. There are numerous antibodies approved for the treatment of diseases including cancer, and many more are in clinical trials. These antibody therapeutics are used in much the same way as injected small-molecule chemotherapeutics. Typical antibodies for cancer are Rituxan, which binds to the CD20 molecule on B cells, and Herceptin, which binds the Her2/neu epidermal growth factor receptor on breast cancer cells (Cragg M S, et al. Blood. 2003 Feb. 1; 101(3):1045-52; and Albanell J, et al. Adv Exp Med Biol. 2003; 532:253-68).

Radioimmunotherapy provides further examples of the successful use of antibodies in cancer therapeutics. Radiolabeled antibodies have the advantage that they can be effective even in the face of defective host immune effector function (Press O W. Semin Oncol. 2003 April; 30 (2 Suppl 4):10-21). Some potentially useful antibodies which have been conjugated to metal chelates for radioimmunotherapy include antibodies HMFG1 (Nicholson, S; et al. Oncology Reports 5, 223-226 (1998)), L6 (DeNardo, S J; et al., Journal of Nuclear Medicine 39, 842-849 (1998)), and Lym-1 (DeNardo, G L; et al., Clinical Cancer Research, 3: 71-79 (1997)). Two radiolabeled monoclonal antibodies that have been approved by the FDA for targeted radiotherapy of lymphoma (Campbell P, et al., Blood Rev. 2003; 17(3): 143-52; and Silverman D H, et al., Cancer Treat Rev. 2004; 30(2): 165-72) are ⁹⁰Y-labeled Zevalin, an IgG that targets CD20 (Li H, et al., J Biol Chem. 2003; 278(43): 42427-34; and Witzig T E, et al., J Clin Oncol. 2002 May 15; 20(10):2453-63), and ¹³¹I-labeled Bexxar, another IgG that targets CD20.

Unfortunately despite the successful use of radiation-delivery antibody vehicles such as Zevalin and Bexxar, these antibody based drugs have serious shortcomings. For example, both are whole IgG molecules that remain in the circulation for days: they pass through the highly radiation-sensitive bone marrow throughout this period, and bone-marrow toxicity limits the dose of radiation that can be tolerated by patients.

In the process of “decorating” the antibody with a therapeutic or diagnostic agent, it is often the case that amino acids essential for target recognition and/or binding affinity are derivatized by the incoming reactive therapeutic agent. Alternatively, an amino acid is derivatized, which prompts an allosteric or other change in the polypeptide structure that diminishes target recognition and/or binding affinity and/or Fc function and/or antibody stability. Thus, derivatization of antibodies with a therapeutic agent or diagnostic agent can result in conjugates that either do not recognize the desired target or do not bind to the target with sufficient affinity. The derivatization of amino acids that play either a minor or no role in target recognition and binding endures as a key difficulty in the wide-spread development and use of antibody conjugates as therapeutics and diagnostics.

Thus, there remains a need in the art for target specific molecules that bind strongly to their target, saturate the target at a 1:1 antibody:target ratio, and which carry a conjugated therapeutic or diagnostic moiety without affecting native antibody Fc activity or stability.

### SUMMARY OF THE INVENTION

The present invention provides a novel conjugate between a polypeptide and a therapeutic or diagnostic agent. The conjugates of the invention have a higher drug antibody ratio (DAR) and superior physiochemical properties, biologic activity, and manufacturability compared to classical antibody drug conjugates with a similar DAR. For simplicity, the invention is described herein in terms of the therapeutic agent conjugate, however, it will be apparent to those of skill in the art that the concept underlying the present invention is equally applicable to a conjugate with a diagnostic agent.

In an exemplary embodiment, the invention provides a conjugate between an antibody and a therapeutic agent. In an exemplary embodiment, the conjugate comprises: (a) a polypeptide therapeutic agent conjugation module (B) to which one or more of the therapeutic agent molecule (A) is covalently bound; and (b) a polypeptide targeting module (C) covalently bound to the therapeutic agent conjugation module.

In various embodiments, the therapeutic agent (A) is a small molecule or protein toxin, the drug conjugation module (B) is a polypeptide that retains favorable physico-chemical properties on chemical conjugation of the drug (A). The targeting module (C) is a polypeptide capable of recognizing and specifically binding target cells of interest.

In various embodiments, one or more component of the conjugate is linked via one or more linkers capable of undergoing cleavage under biologically relevant conditions. In an exemplary embodiment, the cleavage occurs after uptake by a cell, e.g., by endocytosis. In an exemplary embodiment, the cleavage releases the free therapeutic moiety (e.g., free of the linker and components B and C), or it releases a therapeutic moiety bearing the linker (or a fragment of the linker) from the other components (B and C) of the conjugate.

The protein drug conjugate can be categorized into 2 structurally distinct families: PDC1 and PDC2 each comprising A-B-C modules. In an exemplary embodiment of PDC1, none of the amino acids comprising the targeting/binding regions in the targeting protein module (C) are directly conjugated to any drug molecule. In PDC2, some of the amino acids comprising the targeting/binding regions in the targeting protein module (C) may also be directly conjugated to some drug molecules, while the majority of drug molecules are conjugated to the drug conjugation module (B).

In an exemplary embodiment, PDC1 and PDC2 are generated as follows. PDC1: The therapeutic agent payload (A) is synthetically coupled or conjugated to the therapeutic agent conjugation module (B) to form (A-B) which is synthetically fused to the targeting protein module (C) to form A-B-C: Therapeutic agent (A) conjugation is specific to (B). PDC2: The therapeutic agent conjugation module (B) is genetically fused to a targeting module (C) to form B-C which is then conjugated to the therapeutic agent payload (A) to form PDC2: A-B-C. Therapeutic agent (A) conjugation is specific to (B-C). Conjugation of therapeutic agent payload (A) to either compound (B) or (B-C) is capable of achieving a high therapeutic agent to antibody ratio (DAR) with an average number (>0.5) therapeutic agent (A) per B or an average number (>0.5) therapeutic agent (A) per B-C.

In addition to the conjugates, the present invention provides methods of using the conjugates. For example, in one embodiment, there is provided a method of treating a disease in a subject in need of such treatment, said method comprising: administering to said subject a therapeutically effective amount of a conjugate of the invention.

In various embodiments, the invention provides a method of diagnosing a disease by detecting a disease marker in a sample. The method includes: contacting said sample with a conjugate between an antibody and a detectable agent, said conjugate comprising: (a) a polypeptide detectable agent conjugation module (B) to which one or more of detectable agent (A) molecule is covalently bound; and (b) a polypeptide targeting module (C) covalently bound to the detectable agent conjugation module, and determining whether the antibody binds to the marker in the sample by detecting the detectable agent.

Other objects, advantages and embodiments of the invention will be apparent from the detailed description that follows.

### BRIEF DESCRIPTION OF THE DRAWINGS

FIG. 1 depicts a sequence of Albumin Domain 3, comprising of residues from position 381 (starting at VEE . . . ) to 585 (ending at . . . LGL). The 24 lysine residues in this 205 residue protein sequence are boxed.

FIG. 2 depicts a 3-dimensional structure of Albumin Domain III modeled based on the structure of Human Serum Albumin (PDB ID: 1AO6). The Lys residue sidechains are shown using “stick” representation and “ribbon” representation is employed for the Domain III backbone.

FIG. 3 depicts a schematic of a one armed antibody (OAA). It comprises of a heterodimeric Fc fused to a single Fab arm. The first heavy chain (in blue) comprises of the VH, CH1, hinge, CH2 and CH3 domains. The VH and CH1 domains of the heavy chain are paired to the VL and CL domains of the light chain correspondingly. The second heavy chain begins in the upper hinge region (EPKSSDKTHTCPPCPAPELLGGPS . . . ) and the N-termini can be cysteine or other amino acid permitting site specific ligation to the therapeutic agent protein module AB. The OAA can be employed as a targeting module in the antibody therapeutic agent conjugate PDC1.

FIG. 4 depicts a schematic of a one armed antibody (targeting module) fused to a therapeutic agent conjugated albumin domain III (therapeutic agent conjugation module).

FIG. 5 depicts pre-functionalization of conjugation module protein (B) for subsequent conjugation to targeting module (C) using the following reagents and conditions: a) 0.2M solution of 10 (Quanta Biodesign, Ltd, PN 11135, see FIG. 8) in DMF, 1 drop TEA, 0.2M solution of 11 in DMF; b) hydrazine (2 equivalents) in THF at room temperature; c) 14, 30-50 mM 5′-pyridylphosphate, then 1.1 equivalent of 13. (Based on Scheck, R. A.; Dedeo, M. T.; Iavarone, A. T.; Francis, M. B., Optimization of a Biomimetic Transamination Reaction. Journal of the American Chemical Society 2008, 130(35):11762-11770). In one embodiment, the conjugation of a therapeutic agent toxin molecule such as Maytansine to the prefunctionalized conjugation module protein (B) (15 in FIG. 5 above) can be achieved in FIG. 6.

FIG. 6 depicts conjugation of therapeutic agent to conjugation module (B) using the following reagents and conditions: a) Glutaric anhydride, followed by NHS, HOBt, DCC; b) 20 equivalents of 17, 1 equivalent of 15, pH 7.5 phosphate buffer. Ll and L2 defined in FIG. 5. The final coupling of targeting module protein (C) to conjugated module (A-B, compound 18) is achieved as set forth in FIG. 7.

FIG. 7 depicts conjugation of (C) to (A-B) with the following reagents and conditions: a) 0.1M solution of 18 in water at 0° C. treated with pre-cooled 0.1 M HCl (1.5 eq., 0° C.), 30 minutes, then pH adjusted to 3.5 with sodium ascorbate (1M); b) 19, 0.1M in degassed sodium ascorbate buffer, 500 mM LiCl, 0-15° C. L₁, L₂ and R₄ defined in FIG. 5.

FIG. 8 depicts preparation of the cysteine capture reagent. The cysteine capture reagent (11 in FIG. 5) can be obtained as shown in FIG. 8, employing the following reagents and conditions for the intermediate steps: a) n-BuLi, then methyl chloroformate; b) toluene, 170° C.; c) benzylbromoacetate, K₂CO₃, acetone; d) H₂, Pd/C, EtOAc; e) immobilized carbodiimide, NHS; 0 add a solution of 8 to 20 equivalents of 9 in THF, room temperature.

FIG. 9 depicts SDS-PAGE of v5110 and v6265 after purification by protein A and/or SEC gel filtration.

FIG. 10 depicts the UPLC-SEC gel filtration profile of v5110.

FIGS. 11A and 11B depict internalization and cell surface accumulation of antibody and antibody fusion on JIMT-1 and SK-OV3 cells.

FIG. 12 depicts the chemical structure of SMCC-DM1.

FIG. 13A depicts HIC-HPLC profile of unconjugated v5110 and conjugated v5110 (v10135). FIG. 13B depicts SEC-HPLC profile of conjugated v5110 and unconjugated v5110 (v10135).

FIG. 14 depicts deconvoluted LC-MS spectra of conjugated v9992 (v10134).

FIG. 15 depicts regional analysis of conjugation ratios in v10135 and v10134. FIG. 15A shows the regional analysis of conjugation ratios in v10135 where region A (light chain) shows a DAR of 0.53; region B (N-terminal) shows a DAR of 0.53; region C (C-terminal) shows a DAR of 0.33; region D (C-terminal) shows a DAR of 0.33; and region E (N-terminal, HSAdIII) shows a DAR of 0.58. FIG. 15B shows the regional analysis of conjugation ratios in v10134 where region F (Light Chain) shows a DAR of 0.63; region G (N-terminal) shows a DAR of 0.39; region H (C-terminal) shows a DAR of 0.58; region I (C-terminal) shows a DAR of 0.48; and region J (N-terminal) no labelling observed.

FIG. 16 depicts growth inhibition of JIMT-1 cells by conjugated v10134 and v10135.

FIG. 17 depicts representations of the different variants described herein where A represents the Trastuzumab Fab; B represents the Fc; C represents Albumin domain III, fused to Fc; D represents Trastuzumab Fab decorated with DM-1; E represents Fc decorated with DM-1; F represents Albumin domain III decorated with DM-1, fused to Fc; G represents Albumin domain III, and H represents Albumin domain III decorated with DM-1.

FIG. 18 depicts the thermal stability of the constructs determined by using differential scanning calorimetry.

FIG. 19 depicts exemplary polynucleotide sequences of use in expressing the polypeptides of the invention.

FIG. 20 provides amino acid sequences of polypeptides of use in the conjugates of the invention.

### DETAILED DESCRIPTION OF THE INVENTION

### Introduction

Site-specific and target-oriented delivery of therapeutic agents is desirable for the purpose of treating a wide variety of human diseases, such as different types of malignancies and certain neurological disorders. Such procedures are accompanied by fewer side effects and a higher efficacy of therapeutic agent. Various principles have been relied on in designing these delivery systems. For a review, see Garnett, Advanced Drug Delivery Reviews 53:171-216 (2001).

One important consideration in designing a drug delivery system is to target tissues specifically. The discovery of tumor surface antigens has made it possible to develop therapeutic approaches where tumor cells displaying definable surface antigens are specifically targeted and killed. There are three main classes of therapeutic monoclonal antibodies (antibody) that have demonstrated effectiveness in human clinical trials in treating malignancies: (1) unconjugated MAb (monoclonal antibody), which either directly induces growth inhibition and/or apoptosis, or indirectly activates host defense mechanisms to mediate antitumor cytotoxicity; (2) drug-conjugated MAb, which preferentially delivers a potent cytotoxic toxin to the tumor cells and therefore minimizes the systemic cytotoxicity commonly associated with conventional chemotherapy; and (3) radioisotope-conjugated MAb, which delivers a sterilizing dose of radiation to the tumor. See review by Reff et al., Cancer Control 9:152-166 (2002).

In order to arm MAbs with the power to kill malignant cells, the MAbs can be connected to a toxin, which may be obtained from a plant, bacterial, or fungal source, to form chimeric proteins called immunotoxins. Frequently used plant toxins are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325 (2001). As set forth herein, the present invention provides target therapeutic agents.

The conjugates and methods discussed in the following sections are generally representative of the conjugates of the invention and the methods in which such compositions can be used. The following discussion is intended as illustrative of selected aspects and embodiments of the present invention and it should not be interpreted as limiting the scope of the present invention.

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

In describing the present invention, the following terms will be employed, and are intended to be defined as indicated below.

### Abbreviations

“One Armed Antibody” (“OAA”); Protein Drug Conjugate 1 and 2 (“PDC1” and “PDC2”).

### DEFINITIONS

The symbol “R”, as used herein, refers to moiety which is a member selected from the moieties defined in the following section, e.g., substituted or unsubstituted alkyl, substituted or unsubstituted heteroalkyl, substituted or unsubstituted aryl, substituted or unsubstituted heteroaryl, etc. as well as those groups set forth as substituents of these moieties.

Where chemical moieties are specified by their conventional chemical formulae, written from left to right, they optionally equally encompass the moiety which would result from writing the structure from right to left, e.g., —CH₂O— is intended to also recite —OCH₂—; —NHS(O)₂— is also intended to optionally represent. —S(O)₂HN—, etc. Moreover, where compounds can be represented as free acids or free bases or salts thereof, the representation of a particular form, e.g., carboxylic or sulfonic acid, also discloses the other form, e.g., the deprotonated salt form, e.g., the carboxylate or sulfonate salt. Appropriate counterions for salts are well-known in the art, and the choice of a particular counterion for a salt of the invention is well within the abilities of those of skill in the art. Similarly, where the salt is disclosed, this structure also discloses the compound in a free acid or free base form. Methods of making salts and free acids and free bases are well-known in the art.

“A component of a reactive functional group” refers to a leaving group or to a component of the reactive functional group that is itself reactive. Exemplary leaving groups include halogens of an acyl or alkyl halide, the alcohol component of an ester (e.g., an active ester, e.g., N-hydroxysuccinimide), an imidazole and the like. An exemplary reactive component of the reactive functional group is an unsaturated bond (e.g., the double bond of a maleimide, or the unsaturated bond of an alkyne). Additional exemplary components include those forming bonds through coupling reactions (e.g., oxidative coupling, e.g., S—S bond formation).

“Activated derivatives of carboxyl moieties,” and equivalent species, refers to moiety on a precursor component of a conjugate of the invention (e.g., therapeutic agent, polypeptide, linker) having a leaving group, e.g., an active ester, acyl halide, acyl imidazole, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include mono-, di- and multivalent radicals, having the number of carbon atoms designated (i.e., C₁-C₁₀ means one to ten carbons). Examples of saturated alkyl radicals include, but are not limited to, groups such as methyl, methylene, ethyl, ethylene, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, optionally, those derivatives of alkyl defined in more detail below, such as “alkenyl”, “alkynyl”, “alkyldiyl”, “alkyleno” and “heteroalkyl.”

“Alkenyl”, refers to an unsaturated branched, straight-chain or cyclic alkyl radical having at least one carbon-carbon double bond derived by the removal of one hydrogen atom from a single carbon atom of a parent alkene. The radical may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc., and the like. In exemplary embodiments, the alkenyl group is (C₂-C₆)alkenyl.

“Alkynyl” refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-3-yn-1-yl, etc., and the like. In exemplary embodiments, the alkynyl group is (C₂-C₆)alkynyl.

“Alkyldiyl”, refers to a saturated or unsaturated, branched, straight-chain or cyclic divalent hydrocarbon radical derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyls include, but are not limited to methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methany dene-propan-1,1-diyl, buta-1,3-dien-1,1-diyl, buta-1,3-dien-1,3-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. In preferred embodiments, the alkyldiyl group is (C₂-C₆)alkyldiyl. Also preferred are saturated acyclic alkanyldiyl radicals in which the radical centers are at the terminal carbons, e.g., methandiyl(methano); ethan-1,2-diyl(ethano); propan-1,3-diyl(propano); butan-1,4-diyl(butano), and the like (also referred to as alkylenos, defined infra).

“Alkyleno”, refers to a straight-chain alkyldiyl radical having two terminal monovalent radical centers derived by the removal of one hydrogen atom from each of the two terminal carbon atoms of straight-chain parent alkane, alkene or alkyne. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, but[1,3]diyno, etc., and the like. Where specific levels of saturation are intended, the nomenclature alkano, alkeno and/or alkyno is used. In preferred embodiments, the alkyleno group is (C₂-C₆)alkyleno.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si, P and S, and wherein the nitrogen and sulfur atoms may optionally be oxidized and the nitrogen heteroatom may optionally be quaternized. The heteroatom(s) O, N, S, P and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Also included are di- and multi-valent species such as “cycloalkylene.” Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, species such as trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quatemized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Also included are di- and multi-valent linker species, such as “arylene.” Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes aryl and, optionally, heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Exemplary substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, SO₃R′, —NR′—C(O)NR″R′″, —NR″ C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. Accordingly, from the above discussion of substituents, one of skill in the art will understand that the terms “substituted alkyl” and “heteroalkyl” are meant to include groups that have carbon atoms bound to groups other than hydrogen atoms, such as haloalkyl (e.g., —CF₃ and —CH₂CF₃) and acyl (e.g., —C(O)CH₃, —C(O)CF₃, —C(O)CH₂OCH₃, and the like).

The substituents set forth in the paragraph above are referred to herein as “alkyl group substituents.”

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″ C(O)₂R′, —NR—C(NR′R″)═NR′″, —S(O)R′, —S(O)₂R′, SO₃R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R″″)_{d}—, where s and d are independently integers of from 0 to 3, and X is —O—, —NR′—, —S—, —S(O)—, —S(O)₂—, or —S(O)₂NR′—. The substituents R, R′, R″ and R″ are preferably independently selected from hydrogen or substituted or unsubstituted (C₁-C₆)alkyl.

The substituents set forth in the two paragraphs above are referred to herein as “aryl group substituents.”

A “linkage fragment” is a bond, or is a group that is formed by reaction of two reactive functional groups of complementary reactivity. An exemplary linkage fragment is an amide formed by the reaction of an amine and an activated derivative of a carboxylic acid (e.g., acyl halide, acyl imidazole, active ester, etc.). The polypeptide-therapeutic agent conjugates of the invention can be conjugated directly through a linkage fragment or through a linker that includes one or more linkage fragment. For example, a conjugate in which the therapeutic agent is bound to the polypeptide through a linker optionally includes a linkage fragment joining the linker and the therapeutic agent and/or joining the linker and the polypeptide.

The term “Linker” or “L”, as used herein, refers to a single covalent bond or a series of stable covalent bonds incorporating 1-40, e.g., 10-30 nonhydrogen atoms selected from the group consisting of C, N, O, S and P that covalently attach the therapeutic agent to another moiety such as a chemically reactive group or a biological or non-biological component, e.g., the polypeptide of the conjugates of the invention. Exemplary linkers include one or more linkage fragment, e.g., —C(O)NH—, —C(O)O—, —NH—, —S—, —O—, joining the therapeutic agent to the linker and/or the linker to the polypeptide and the like. Linkers are also of use to join the therapeutic agent “core structure” to a reactive functional group, or a component of a reactive functional group.

The term “antibody” herein is used in the broadest sense and specifically covers monoclonal antibodies, polyclonal antibodies, multispecific antibodies (e.g., bispecific antibodies), and antibody fragments, so long as they exhibit the desired biological activity. Antibodies may be murine, human, humanized, chimeric, or derived from other species.

An antibody is a protein generated by the immune system that is capable of recognizing and binding to a specific antigen. (Janeway, et al (2001) “Immunobiology”, 5th Ed., Garland Publishing, New York). A target antigen generally has numerous binding sites, also called epitopes, recognized by CDRs (complementary determining regions) on multiple antibodies. Each antibody that specifically binds to a different epitope has a different structure. Thus, one antigen may have more than one corresponding antibody.

The term “antibody,” as used herein, also refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

“Antibody fragments” comprise a portion of a full length antibody, generally the antigen binding or variable region thereof. Examples of antibody fragments include Fab, Fab′, F(ab′).sub.2, and Fv fragments; diabodies; linear antibodies; fragments produced by a Fab expression library, anti-idiotypic (anti-Id) antibodies, CDR, ECD (extracellular domain), and epitope-binding fragments of any of the above which immunospecifically bind to cancer cell antigens, viral antigens or microbial antigens, single-chain antibody molecules; and multispecific antibodies formed from antibody fragments.

An “intact antibody” herein is one comprising VL and VH domains, as well as complete light and heavy chain constant domains.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see, U.S. Pat. No. 4,816,567). The “monoclonal antibodies” may also be isolated from phage antibody libraries using the techniques described in Clackson et al (1991) Nature, 352:624-628; Marks et al (1991) J. Mol. Biol., 222:581-597; for example.

As used herein, the term “chimeric antibody” refers to a monoclonal antibody comprising a variable region, i.e. binding region, from one source or species and at least a portion of a constant region derived from a different source or species, usually prepared by recombinant DNA techniques. Chimeric antibodies comprising a murine variable region and a human constant region are especially preferred in certain applications of the invention, particularly human therapy, because such antibodies are readily prepared and may be less immunogenic than purely murine monoclonal antibodies. Such murine/human chimeric antibodies are the product of expressed immunoglobulin genes comprising DNA segments encoding murine immunoglobulin variable regions and DNA segments encoding human immunoglobulin constant regions. Chimeric monoclonal antibodies may have specificity toward a tumor associated antigen. Other forms of chimeric antibodies encompassed by the invention are those in which the class or subclass has been modified or changed from that of the original antibody. Such “chimeric” antibodies are also referred to as “class-switched antibodies”. Methods for producing chimeric antibodies involve conventional recombinant DNA and gene transfection techniques now well known in the art. See, e.g., Morrison, S. L, et al., Proc. Nat'l Acad. Sci., 81, 6851 (1984).

Encompassed by the term “chimeric antibody” is the concept of “humanized antibody”, that is those antibodies in which the framework or “complementarity determining regions (“CDR”) have been modified to comprise the CDR of an immunoglobulin of different specificity as compared to that of the parent immunoglobulin. In an exemplary embodiment, a murine CDR is grafted into the framework region of a human antibody to prepare the “humanized antibody”. See, e.g., L. Riechmann et al., Nature 332, 323 (1988); M. S, Neuberger et al., Nature 314, 268 (1985). Exemplary CDRs correspond to those representing sequences recognizing the antigens noted above for the chimeric and bifunctional antibodies (EPA 0 239 400), incorporated herein by reference, for its teaching of CDR modified antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. See e.g., Cabilly U.S. Pat. No. 4,816,567; Queen et al. (1989) Proc. Nat'l Acad. Sci. USA 86:10029-10033; and Antibody Engineering: A Practical Approach (Oxford University Press 1996).

As used herein, the terms “specific”, “specifically binds” and “binds specifically” refer to the selective binding of the antibody to the target antigen epitope. Antibodies can be tested for specificity of binding by comparing binding to appropriate antigen to binding to irrelevant antigen or antigen mixture under a given set of conditions. If the antibody binds to the appropriate antigen at least 2, 5, 7, and preferably 10 times more than to irrelevant antigen or antigen mixture then it is considered to be specific. In one embodiment, a specific antibody is one that only binds the HER2/neu antigen, but does not bind to the irrelevant antigen. In another embodiment, a specific antibody is one that binds human HER2/neu antigen but does not bind a non-human HER2/neu antigen with 70%, 75%, 80%, 85%, 90%, 91%, 92%, 93%, 94%, 95%, 96%, 97%, 98%, 99% or greater amino acid homology with the HER2/neu antigen. In another embodiment, a specific antibody is one that binds human HER2/neu antigen and binds murine HER2/neu antigen, but with a higher degree of binding the human antigen. In another embodiment, a specific antibody is one that binds human HER2/neu antigen and binds primate HER2/neu antigen, but with a higher degree of binding the human antigen. In another embodiment, the specific antibody binds to human HER2/neu antigen and any non-human HER2/neu antigen, but with a higher degree of binding the human antigen or any combination thereof.

The terms “treat” and “treatment” refer to both therapeutic treatment and prophylactic or preventative measures, wherein the object is to prevent or slow down (lessen) an undesired physiological change or disorder, such as the growth, development or spread of a hyperproliferative condition, such as cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

“Hyperproliferative disorder” is indicated by tumors, cancers, and neoplastic tissue, including pre-malignant and non-neoplastic stages, and also include psoriasis, endometriosis, polyps and fibroadenoma.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “disease” is a state of health of an animal wherein the animal cannot maintain homeostasis, and wherein if the disease is not ameliorated then the animal's health continues to deteriorate.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid nucleic acid encoding additional polypeptide sequence.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides. The term “oligonucleotide” typically refers to short polynucleotides, generally no greater than about 50 nucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a nucleic acid sequence encodes a protein if transcription and translation of mRNA corresponding to that nucleic acid produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that nucleic acid or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCCS' and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J Mol. Biol. 215:403-410), and can be accessed, for example at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

A “heterologous nucleic acid expression unit” encoding a polypeptide is defined as a nucleic acid having a coding sequence for a polypeptide of interest operably linked to one or more expression control sequences such as promoters and/or repressor sequences wherein at least one of the sequences is heterologous, i.e., not normally found in the host cell.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a nucleic acid is able to promote transcription of the coding region.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

A “genetically engineered” or “recombinant” cell is a cell having one or more modifications to the genetic material of the cell. Such modifications are seen to include, but are not limited to, insertions of genetic material, deletions of genetic material and insertion of genetic material that is extrachromasomal whether such material is stably maintained or not.

A “peptide” is an oligopeptide, polypeptide, peptide, protein or glycoprotein. The use of the term “peptide” herein includes a polypeptide having a sugar molecule attached thereto when a sugar molecule is attached thereto.

As used herein, “native form” means the form of the polypeptide when produced by the cells and/or organisms in which it is found in nature. When the polypeptide is produced by a plurality of cells and/or organisms, the polypeptide may have a variety of native forms.

“Peptide” refers to a polymer in which the monomers are amino acids and are joined together through amide bonds, alternatively referred to as a polypeptide. Additionally, unnatural amino acids, for example, β-alanine, phenylglycine and homoarginine are also included. Amino acids that are not nucleic acid-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer thereof. The L-isomer is generally preferred. In addition, other peptidomimetics are also useful in the present invention. As used herein, “peptide” refers to both glycosylated and unglycosylated polypeptides. Also included are polypeptides that are incompletely glycosylated by a system that expresses the polypeptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “parent sequence” has the meaning normally ascribed to it in the art. In various embodiments, the parent sequence of an antibody of use in the invention is the sequence of a known antibody. In various embodiments, the known antibody is an antibody undergoing clinical testing for use in humans or other animals, is approved for use in humans or other animals and/or is commercially available. In certain embodiments, the parent antibody is a “hit” or lead in a discovery process, which can be further optimized according to the present invention to introduce other properties of interest. For instance, a parent antibody will bind the target of interest but might require humanization or other kinds of optimization. According to certain embodiments, single or multiple amino acid substitutions (in certain embodiments, conservative amino acid substitutions) may be made in the parent sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid does not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

The term “peptide conjugate,” refers to species of the invention in which a polypeptide is conjugated with a drug, toxin or other therapeutic or diagnostic species as set forth herein.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, (-carboxyglutamate, and O-phosphoserine. Amino acid analogs refers to compounds that have the same basic chemical structure as a naturally occurring amino acid, i.e., an a carbon that is linked to a hydrogen, a carboxyl group, an amino group, and an R group, e.g., homoserine, norleucine, methionine sulfoxide, methionine methyl sulfonium. Such analogs have modified R groups (e.g., norleucine) or modified polypeptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

It will be appreciated, of course, that the polypeptides may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of polypeptide chemistry which will not adversely affect the in vivo activities of the polypeptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm), Fmoc or Boc groups. Desamino analogs of amino acids are also useful N-terminal blocking groups, and can either be coupled to the N-terminus of the polypeptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the polypeptide's C-terminal residue or used in place of it. Further, it will be appreciated that the free amino and carboxyl groups at the termini can be removed altogether from the polypeptide to yield desamino and descarboxylated forms thereof without affect on polypeptide activity.

Other modifications can also be incorporated without adversely affecting the activity and these include, but are not limited to, substitution of one or more of the amino acids in the natural L-isomeric form with amino acids in the D-isomeric form. Thus, the polypeptide may include one or more D-amino acid resides, or may comprise amino acids which are all in the D-form. Retro-inverso forms of polypeptides in accordance with the present invention are also contemplated, for example, inverted polypeptides in which all amino acids are substituted with D-amino acid forms.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99% identity. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Also comtemplated are short insertions and/or deletions. Insertions and/or deletions can be particularly effective at the N- and/or C-terminal ends, which are involved in the creation of protein fusions of the invention. Insertions and/or deletions can be based on sequences in the parent sequence when fragments of the parent protein sequence are being contemplated. Thus, the foregoing examples demonstrate that those of skill in the art can recognize sequence motifs and structural conformations that may be used to define structural and functional domains in accordance with the invention.

The term “water-soluble” refers to moieties that have some detectable degree of solubility in water. Methods to detect and/or quantify water solubility are well known in the art. Exemplary water-soluble polymers include polypeptides, saccharides, poly(ethers), poly(amines), poly(carboxylic acids) and the like. polypeptides can have mixed sequences or be composed of a single amino acid, e.g. poly(lysine). Similarly, saccharides can be of mixed sequence or composed of a single saccharide subunit, e.g., dextran, amylose, chitosan, and poly(sialic acid). An exemplary poly(ether) is poly(ethylene glycol). Poly(ethylene imine) is an exemplary polyamine, and poly(aspartic) acid is a representative poly(carboxylic acid).

“Poly(alkylene oxide)” refers to a genus of compounds having a polyether backbone. Poly(alkylene oxide) species of use in the present invention include, for example, straight- and branched-chain species. Moreover, exemplary poly(alkylene oxide) species can terminate in one or more reactive, activatable, or inert groups. For example, poly(ethylene glycol) is a poly(alkylene oxide) consisting of repeating ethylene oxide subunits, which may or may not include additional reactive, activatable or inert moieties at either terminus. Useful poly(alkylene oxide) species include those in which one terminus is “capped” by an inert group, e.g., monomethoxy-poly(alkylene oxide). When the molecule is a branched species, it may include multiple reactive, activatable or inert groups at the termini of the alkylene oxide chains and the reactive groups may be either the same or different. Derivatives of straight-chain poly(alkylene oxide) species that are heterobifunctional are also known in the art.

The terms “targeting module”, “targeting moiety” and “targeting agent”, as used herein, refer to species that will selectively localize in a particular tissue or region of the body. The localization is mediated by specific recognition of molecular determinants, molecular size of the targeting agent or conjugate, ionic interactions, hydrophobic interactions and the like. Other mechanisms of targeting an agent to a particular tissue or region are known to those of skill in the art. In an exemplary embodiment, a “targeting module” is an antibody, e.g., an antibody of a conjugate of the invention.

As used herein, “therapeutic agent” means any agent useful for therapy including, but not limited to, antibiotics, anti-inflammatory agents, anti-tumor drugs, cytotoxins, and radioactive agents. “Therapeutic agent” includes prodrugs of bioactive agents, constructs in which more than one therapeutic agent is linked to a carrier, e.g., multivalent agents. Therapeutic moiety also includes peptides, and constructs that include peptides. “Therapeutic agent” thus means any agent useful for therapy including, but not limited to, antibiotics, anti-inflammatory agents, anti-tumor drugs, cytotoxins, and radioactive agents. “Therapeutic agent” includes prodrugs of bioactive agents, constructs in which more than one therapeutic moiety is linked to a carrier, e.g., multivalent agents. The targeting module of the constructs of the invention can also function as a therapeutic agent, for example, when the targeting module is an antibody that can bind to an antigen and regulate function. An exemplary therapeutic agent is diptheria toxin.

As used herein, “anti-tumor drug” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, interferons and radioactive agents. Also encompassed within the scope of the term “anti-tumor drug,” are conjugates of peptides with anti-tumor activity, e.g. TNF-α. Conjugates include, but are not limited to those formed between a therapeutic protein and an antibody of the invention. A representative conjugate is that formed between an antibody of the invention, e.g, the antibody portion of PDC1, and TNF-α.

As used herein, “a cytotoxin or cytotoxic agent” means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof. Other toxins include, for example, ricin, CC-1065 and analogues, the duocarmycins. Still other toxins include diphtheria toxin, and snake venom (e.g., cobra venom).

As used herein, “a radioactive agent” includes any radioisotope that is effective in diagnosing or destroying a tumor. Examples include, but are not limited to, indium-111, cobalt-60 and technetium. Additionally, naturally occurring radioactive elements such as uranium, radium, and thorium, which typically represent mixtures of radioisotopes, are suitable examples of a radioactive agent. The metal ions are typically chelated with an organic chelating moiety.

Many useful chelating groups, crown ethers, cryptands and the like are known in the art and can be incorporated into the compounds of the invention (e.g. EDTA, DTPA, DOTA, NTA, HDTA, etc. and their phosphonate analogs such as DTPP, EDTP, HDTP, NTP, etc). See, for example, Pitt et al., “The Design of Chelating Agents for the Treatment of Iron Overload,” In, INORGANIC CHEMISTRY IN BIOLOGY AND MEDICINE; Martell, Ed.; American Chemical Society, Washington, D. C., 1980, pp. 279-312; Lindoy, THE CHEMISTRY OF MACROCYCLIC LIGAND COMPLEXES; Cambridge University Press, Cambridge, 1989; Dugas, BIOORGANIC CHEMISTRY; Springer-Verlag, New York, 1989, and references contained therein.

Additionally, a manifold of routes allowing the attachment of chelating agents, crown ethers and cyclodextrins to other molecules is available to those of skill in the art. See, for example, Meares et al., “Properties of In Vivo Chelate-Tagged Proteins and Polypeptides.” In, MODIFICATION OF PROTEINS: FOOD, NUTRITIONAL, AND PHARMACOLOGICAL ASPECTS;” Feeney, et al., Eds., American Chemical Society, Washington, D. C., 1982, pp. 370-387; Kasina et al., Bioconjugate Chem., 9:108-117 (1998); Song et al., Bioconjugate Chem., 8:249-255 (1997).

As used herein, “pharmaceutically acceptable carrier” includes any material, which when combined with the conjugate retains the activity of the conjugate activity and is non-reactive with the subject's immune system. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, intranasal or subcutaneous administration, intrathecal administration, or the implantation of a slow-release device e.g., a mini-osmotic pump, to the subject.

The term “isolated” refers to a material that is substantially or essentially free from components, which are used to produce the material. For polypeptide conjugates of the invention, the term “isolated” refers to material that is substantially or essentially free from components, which normally accompany the material in the mixture used to prepare the polypeptide conjugate. “Isolated” and “pure” are used interchangeably. Typically, isolated polypeptide conjugates of the invention have a level of purity preferably expressed as a range. The lower end of the range of purity for the polypeptide conjugates is about 60%, about 70% or about 80% and the upper end of the range of purity is about 70%, about 80%, about 90% or more than about 90%.

When the polypeptide conjugates are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity.

Purity is determined by any art-recognized method of analysis (e.g., band intensity on a silver stained gel, polyacrylamide gel electrophoresis, HPLC, or a similar means).

In some embodiments, the drug molecule is selected is selected from anticancer agents, cytotoxic natural products, phytotoxins, radioisotopes, bioactive proteins, enzymes that activate prodrugs of cytotoxic agents and photosensitizers. In some embodiments, the drug molecule belongs to the class of Maytansinoids, Auristatins, Dolastatins, Calicheamicin, Vinca alkaloids etc known in the art [Dosio et al. (2011) Toxins 3:848-883]. In some embodiment the drug molecule (A) belongs to the class of kinase inhibitors, alkylating agents, purine analogues (see: http://www.pharmacology2000.com/Anticancer/classes1.htm). In some embodiments, A can be 100% of a given cytotoxic or a mixture of different cytotoxic agents for broader anti-tumor effects.

### The Embodiments

### The Conjugates

The present invention provides a novel conjugate between a polypeptide and a therapeutic or diagnostic agent. For simplicity, the invention is described herein in terms of the therapeutic agent conjugate, however, it will be apparent to those of skill in the art that the concept underlying the present invention is equally applicable to a conjugate with a diagnostic agent.

In an exemplary embodiment, the invention provides a conjugate between an antibody and a therapeutic agent. In an exemplary embodiment, the conjugate comprises: (a) a polypeptide therapeutic agent conjugation module (B) to which one or more of the therapeutic agent molecule (A) is covalently bound; and (b) a polypeptide targeting module (C) covalently bound to the therapeutic agent conjugation module.

Proteins are often affected by chemical modification resulting in significant change in structure and functional properties of the protein. This is particularly true of the chemical modification of a protein with hydrophobic molecules such as therapeutic agents (e.g., toxins). The design of the modular conjugate described herein mitigates this problem. In an exemplary embodiment, the targeting module comprised the antigen binding domain and the Fc segments of an antibody.

In an exemplary embodiment, A and B modules are conjugated to provide AB (A+B>A−B), which is then conjugated to C (A−B+C>A−B−C; giving PDC1). The method of assembling the conjugate of the invention protects the amino acids of the targeting domain and Fc of (C) from therapeutic agent (A) conjugation, overcoming structural and stability issues intrinsic to traditional antibody therapeutic agent conjugate (ADC) designs.

Similarly, in various embodiments, the conjugation of A to B−C(A+B−C>A−B−C; giving PDC2) wherein the therapeutic agent conjugation module (B) is rich in a residue type required for therapeutic agent (A) conjugation relative to the targeting module (C), protects the targeting module (C) from significant conjugation with (A).

In various embodiments, modules (B) and (C) are co-expressed as a fusion protein. In an exemplary embodiment, C-terminus of module (B) is fused to the N-terminus of module (A). In another exemplary embodiment, the C-terminus of module (A) is fused to the N-terminus of module (B). The fusion protein is then conjugated at the therapeutic agent conjugation module (B) with (A). In an exemplary embodiment, both (B) and (C) are conjugated with (A).

In an exemplary embodiment, the targeting module is essentially free from bound therapeutic agent (e.g., agent bound covalently). In various embodiments, a first fraction of the therapeutic agent is covalently bound to the therapeutic agent conjugation module, and a second fraction of the therapeutic agent is covalently bound to the targeting module. In various embodiments, the first fraction includes relatively more covalently bound therapeutic agent than the second fraction, i.e., the first fraction is more populous than the second fraction.

In various embodiments, the targeting module has covalently bound therapeutic agent conjugated thereto, however, the amount of therapeutic agent covalently bound to the targeting moiety is less than the amount of therapeutic agent covalently bound to the therapeutic agent conjugation module. In an exemplary embodiment in which the polypeptide substrate for conjugate is (B-C), the therapeutic agent conjugation module exerts a protective effect on the targeting module. In an exemplary embodiment, the PDC of the invention (A-B-C) includes fewer moleules of (A) attached to (C) than a conjugate that is structurally identical with the exception that the (B) is absent from the conjugate (“Conjugate B”). In various embodiments, the amount of module (A) bound to module (C) of a PDC of the invention is less than about 80%, less than about 70%, less than about 60% or less than about 50% of the amount bound to module (C) in Conjugate B. In various embodiments, the amount of module (A) bound to module (C) of a PDC of the invention is less than about 80%, less than about 70%, less than about 60% or less than about 50% of the amount bound to module (B).

In an exemplary embodiment, module (C) is the Fc region of an antibody. In an exemplary embodiment, (C) is the Fc region of an antibody and (B) is a domain (DI, DII or DIII) of albumin or is albumin itself. See, e.g., Dockal et al., J. Biol. Chem., 274, 29303-29310 (1999). In an exemplary embodiment, module (B) is a polypeptide fragment derived from albumin (e.g., the aa sequence is a partial albumin sequence). In an exemplary embodiment, (C) is the Fc region of trastuzumab and (B) is Domain III of albumin.

The therapeutic agent can be bound to the conjugate, preferably at module (B), through any useful natural or non-natural amino acid residue of module (B). In exemplary embodiments, the therapeutic agent is bound to the polypeptide through a cysteine or lysine residue of the polypeptide. In various embodiments, the amino acid residue is a cysteine or lysine residue of module (B). In various embodiments, the therapeutic agent (A) is covalently bound to module (B) through a disulfide bond. In various embodiments, the therapeutic agent (A) is covalently bound to module (B) through a bispecific or bifunctional linker.

In an exemplary embodiment, module (B) has a relatively large number of natural amino acids that can be chemically conjugated directly to the therapeutic agent (A) or which can be functionalized and chemically conjugated to the therapeutic agent (A). For example, a useful (B) module includes a sufficient number of amino acids that when the PDC is functionalized with therapeutic agent(s), module (B) retains the pharmaceutical properties of solubility and stability, and preferably does not exhibit a propensity for hydrophobic collapse and/or does not show a high clearance rate when administered in vivo.

In various embodiments module (B-C) has a relatively large number of natural amino acids in domain B that can be chemically conjugated directly to the therapeutic agent (A) or which can be functionalized and chemically conjugated to the therapeutic agent (A). For example, a useful module (B) includes a sufficient number of amino acids that when the PDC is functionalized with therapeutic agent(s), module (B-C) retains the pharmaceutical properties of solubility and stability, and preferably does not exhibit a propensity for hydrophobic collapse or does not show a high clearance rate when administered in vivo.

In an exemplary embodiment, more than one molecule of the therapeutic agent is conjugated to module B. In various embodiments, more than 2, more than 3, more than 4, more than 5, more than 6, or more than 7 molecules of therapeutic agent (A) are conjugated to module (B). In various embodiments, more than one molecule of the therapeutic agent (A) is conjugated to module (B-C). In various embodiments, more than 2, more than 3, more than 4, more than 5, more than 6, or more than 7 molecules of therapeutic agent (A) are conjugated to module (B-C). In an exemplary embodiment, module (B-C) includes a sufficient number of amino acids that when the PDC is functionalized with therapeutic agent(s), it retains desirable properties independent of the number of therapeutic agents (A) conjugated to module (B-C), wherein (A)ₙ(B-C) is selected such that n 1, 2, 3, 4, 5, 6, 7, or more. Exemplary desirable properties include the pharmaceutical properties of solubility and stability. Moreover, the conjugate (A)ₙ(B-C) preferably does not exhibit a propensity for hydrophobic collapse or does not show a high clearance rate when administered in vivo.

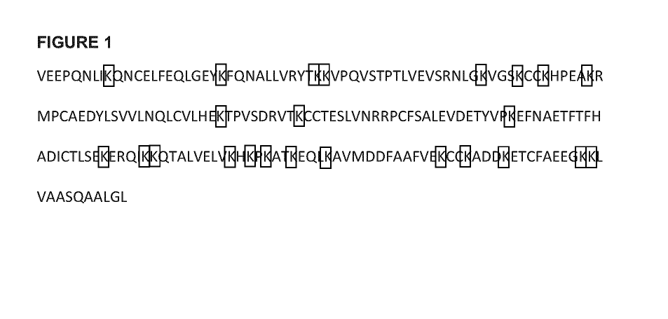

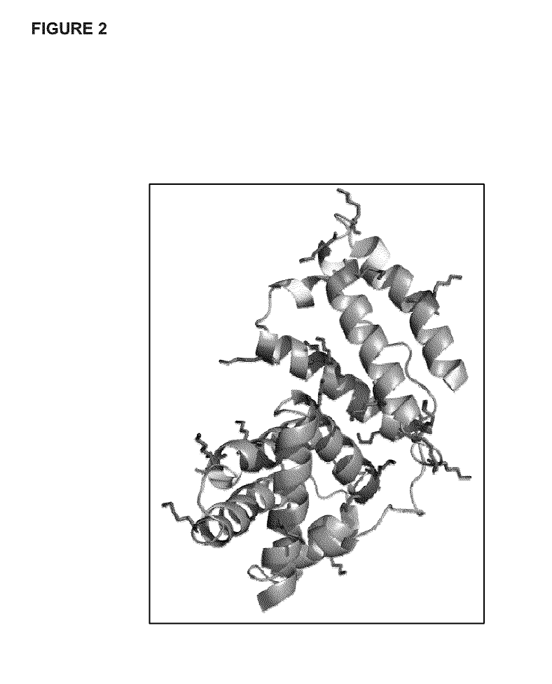

In an exemplary embodiment the therapeutic agent conjugation module (B) comprises a lysine rich polypeptide. In one embodiment the lysine rich therapeutic agent conjugation module (B) comprises domain III of Albumin. An exemplary polypeptide comprising domain III of albumin has the amino acid sequence shown in FIG. 1. An exemplary polypeptide of use in the present invention is derived by splicing the original sequence of wild type human serum albumin and comprises residues from position 381 and is 205 amino acids long in the wild type human serum albumin sequence. Selecting a splice site proximal to position 381 in the original sequence of wild type human serum albumin can provide another exemplary polypeptide of use in the conjugates of the invention. Exemplary conjugation module (B) sequence can be derived from allotypes or mutant forms of human serum albumin. The domain III polypeptide, or a variant thereof, can be acquired by recombinant expression. The structure of albumin domain III is presented in FIG. 2. In an exemplary embodiment, the fusion between Domain III of albumin is at L205 (SEQ. ID. NO.: 14). In an exemplary embodiment, there is a linker attached to L205 (e.g., Ala-Ala). In an exemplary embodiment, the hinge of the Fc is attached to Domain III through E208 (SEQ. ID. NO.: 14).

In various embodiments, the therapeutic agent conjugation module (B) comprises a ubiquitin protein. In one embodiment, the design of PDC1 or PDC2 supports internalization of the antibody therapeutic agent conjugate and facilitates redirection of the therapeutic agent for lysozomal degradation. In some embodiments, the modular protein therapeutic agent conjugate comprises one or more cell penetrating polypeptides (CPPs). In various embodiments, the cell penetrating peptide is from the group of HIV-1 TAT protein [Green and Loewenstein (1988) Cell 55:1179-1188] and polyarginines [Nakase, et al. (2008) Adv Drug Deliv Rev 60:598-607].

In an exemplary embodiment, the modular protein therapeutic agent conjugate of the invention employs non-natural amino acids and their reactions for site-specific conjugation [de Graaf, et al. (2009) Bioconjug Chem 20:1281] in modules (A), (B) or (C).

In an exemplary embodiment, the therapeutic agent and the therapeutic agent conjugation module are conjugated using Click Chemistry (H. C. Kolb, M. G. Finn and K. B. Sharpless (2001). “Click Chemistry: Diverse Chemical Function from a Few Good Reactions”. Angewandte Chemie International Edition 40 (11): 2004-2021). In various embodiments, the therapeutic agent conjugation module reacts using two different types of linker mechanism (“click-chemistries”) available to it. The first type of linker and click chemistry is employed to selectively fuse the therapeutic agents to the respective conjugation module i.e., link (A) to (B) or link (A) to (B-C). The second type of linker and click chemistry is employed to chemically fuse the targeting protein module to the therapeutic agent conjugation module, i.e., link (A-B) to (C).

According to an exemplary embodiment, the therapeutic agent conjugation module (B) is a toxin conjugation module (B′), which is first conjugated to the toxin molecules (A) using click chemistry Reaction 1 and subsequently, the toxin loaded conjugation module is conjugated to the targeting module using click chemistry Reaction 2. A number of chemical species and reagents required to achieve the selective conjugation chemistries is known in the art [Hermanson (2013) Bioconjugation Techniques, 3rd edition, Academic press].

In various embodiments, the therapeutic agent molecule (A) is conjugated to the drug conjugation module (B) or (B-C) using one of the linker chemistries involving creation of disulfide linkage fragment, hydrazone linkage fragment or malemide chemistry based thioether linkage fragment. Linkers formed using such chemistries and precursors to such linkers are known in the art [Ducry and Stump (2010) Bioconjugate Chem 21:5-13].

In various embodiments, the therapeutic agent molecule (A) comprises of a toxin and a linker component. In an embodiment, this toxin is a maytansine and the linker is succinimidyl-4-(N-maleimidomethyl)cyclohexane-1-carboxylate (SMCC).

In one embodiment, the targeting polypeptide module (C) is an antibody or a protein comprising one or more members of a subset of domains present in an antibody. The polypeptide therapeutic agent conjugate therapeutic in this embodiment may be referred to as an “antibody therapeutic agent conjugate therapeutic”. An exemplary targeting polypeptide module comprises an element from the group consisting of a full size antibody such as an IgG, or an antibody fragment, such as a one armed antibody, a half antibody, a Fab domain of an antibody, an scFv or a domain antibody, an Fc domain of an antibody, and an heterodimeric Fc domain of an antibody. As those of skill will appreciate two or more of these antibody fragments can be combined (chemically or through expression of a fusion protein) to form a targeting polypeptide module of use in a conjugate of the invention.

In an exemplary embodiment, the targeting polypeptide module is a one-armed (monovalent) antibody. Exemplary one-armed antibody PDCs (OA-PDC) against certain target antigens have advantages over a larger fragment or full size, bivalent antibody PDC. For example, for an exemplary OA-PDC, cytotoxicity is largely driven by the total intracellular concentration of the drug conjugate. Antibody mediated ADCC, CDC and/or ADCP and/or blocking receptor-ligand function may also contribute when the OA-PDC coats the cell surface. Typically, an OA-PDC is expected to bind receptor at a 1:1 OA-PDC:receptor ratio whereas a full sized-ADC binds receptor at a ratio of 1:2. Using similar stoichiometric reasoning, more surface bound OA-PDC molecules could translate to faster accumulation and potentially higher concentrations of internalized OA-PDC molecules compared to a full sized ADC. In short, the exemplary OA-PDC has advantages of mass action: more antibody decoration, more antibody is internalized/receptor and together with a higher DAR thus reduce the threshold and drug needed to kill a cell.

In various embodiments, the targeting module (C) is a polypeptide that has been matured to recognize and bind to a target receptor with very high affinity and high Bₘₐₓ. In various embodiments, the target cell is a diseased cell and the targeting module recognizes a target receptor or other molecule on the target cell surface. In an exemplary embodiment, the target molecule facilitates the internalization of the protein therapeutic agent conjugated therapeutic into the target cell upon binding of the targeting module to the cell. In an exemplary embodiment, the targeting polypeptide module functions as a “Trojan Horse” for the delivery of the therapeutic agent (A), e.g., a toxin, to the diseased target cell.

In various embodiments, the targeting module (C) is a monovalent antibody construct comprising an antigen-binding polypeptide construct, which monovalently binds an antigen; and a dimeric Fc polypeptide construct comprising two monomeric Fc polypeptides each comprising a CH3 domain, wherein one said monomeric Fc polypeptide is fused to at least one polypeptide from the antigen-binding polypeptide construct. In an exemplary embodiment, the monovalent antibody construct displays an increase in binding density and Bₘₐₓ to a target cell displaying the antigen as compared to a corresponding monospecific bivalent antibody construct with two antigen binding regions. In an exemplary embodiment, the monovalent antibody construct shows superior efficacy and/or bioactivity as compared to the corresponding bivalent antibody construct. In various embodiments, the superior efficacy and/or bioactivity is the result of the increase in binding density and resulting increase in “decoration” of a target cell with a conjugate of the invention. In an exemplary embodiment, the increase in Bₘₐₓ or binding density and resultant increase in target “decoration” by the monovalent antibody construct is the result of specific target binding of the conjugate to the cell and is not due to nonspecific binding of these two species. In certain embodiments the maximum binding occurs at a target to antibody ratio of about 1:1.

In various embodiments, the targeting polypeptide module (C) is a monovalent antibody construct that possesses at least one of the following attributes: increased Bₘₐₓ compared to corresponding monospecific bivalent antibody constructs (FSA); K_{d} comparable to corresponding FSA; same or slower off-rate compared to corresponding FSA; decreased or partial agonism; no cross-linking and/or dimerization of targets; specificity and/or selectivity for target cell of interest; full or partial or no inhibition of target cell growth; complete Fc capable of inducing effector activity; and ability to be internalized by target cell.

In some embodiments, the targeting module (C) is a monovalent antibody construct that possesses at least one of the following minimal attributes: increased Bₘₐₓ compared to corresponding FSA; K_{d} comparable to corresponding FSA; same or slower off-rate compared to corresponding FSA; decreased or partial agonism; no cross-linking and/or dimerization of targets; specificity and/or selectivity for target cell of interest; full or partial or no inhibition of target cell growth; complete Fc capable of inducing effector activity; and optionally the ability to be internalized by target cell.

In an exemplary embodiment, the targeting module (C) is a monovalent internalizing antibody. In some embodiments, module (C) displays one or more of the following efficacy factors: a) the ability of the monovalent antibody construct to be internalized, b) the increased Bₘₐₓ and K_{d} and slow off rate of the monovalent antibody construct, and c) No agonism/partial agonism of the monovalent antibody construct.

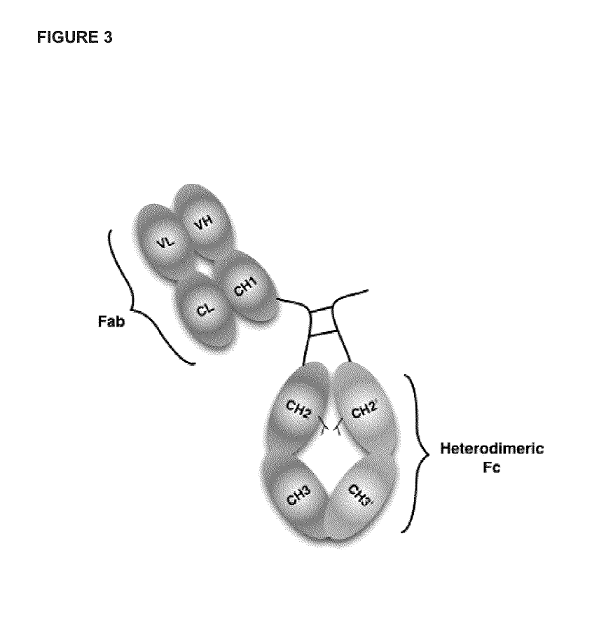

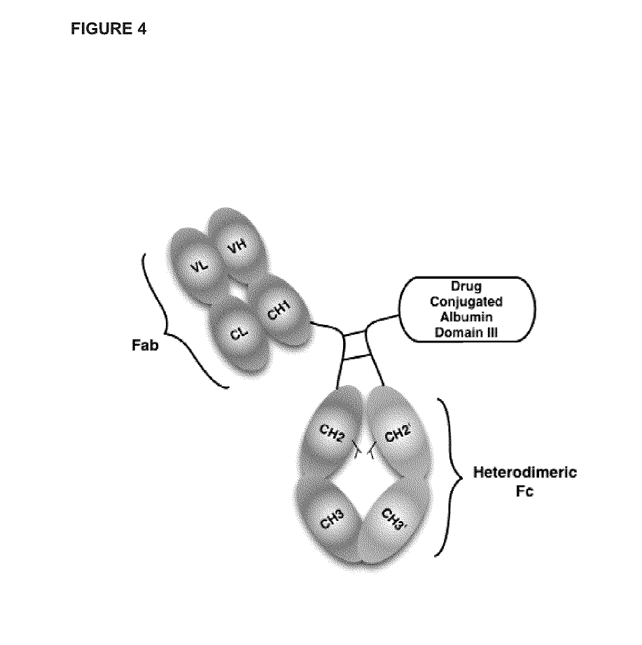

In an exemplary embodiment, a one armed antibody (shown schematically in FIG. 3) is used as the targeting polypeptide module. It comprises of a heterodimeric Fc, with one of the two Fc chains fused to a Fab (fragment antigen binding) via the IgG1 hinge.

In an exemplary embodiment of PDC1, none of the amino acids comprising the targeting/binding regions in the targeting protein module (C) are directly conjugated to any therapeutic agent molecule.

In an embodiment of PDC1, none of the amino acids comprising the Fc regions in the targeting protein module (C) are directly conjugated to any therapeutic agent molecule.

In various embodiments of PDC2, none or few of the amino acids comprising the targeting/binding regions in the targeting protein module (C) are directly conjugated to any therapeutic agent molecule compared to (B). The concept is the lysine rich and hydrophobic species stabilizing domain will be selectively labeled compared to native lysines on the Fc. The chemistry used in forming the PDCs of the invention chemistry here is region-selective.

In various embodiments of PDC2, none or few of the amino acids comprising the Fc regions in the targeting protein module C are directly conjugated to any therapeutic agent molecule compared to (B). Similar to the embodiments, above, the concept is the lysine rich and hydrophobic species stabilizing domain will be selectively labeled compared to native lysines on the Fc, and the chemistry used in forming the PDCs of the invention chemistry here is region-selective.

The invention is exemplified by reference to toxins as exemplary therapeutic agents. The invention can be practiced with any toxin or therapeutic moiety that is derivatizable in a manner that allows placement of a reactive functional group on the core of the moiety or on a linker attached to the core of the moiety. This reactive functional group is of reactivity complementary to that of a reactive functional group on a polypeptide component of the conjugate (e.g., B and/or C). Exemplary toxins of use in the present invention are set forth in Table 1.

TABLE 1
Toxins.
Chemical Structure

| | | | | |
|---|---|---|---|---|
| Toxin Name/ | | | | Activity (IC50 |
| Source/ | CAS RN/ | Indication/ | | nM); Tumor |
| Alternate ID | Analogs | Toxicity | Mechanism | Type |

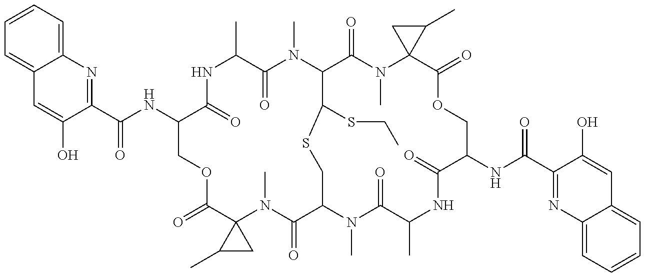 [SMILES: CCSC1SCC2C(=O)N(C)C3(CC3C)C(=O)OCC(NC(=O)c3nc4ccccc4cc3O)C(=O)NC(C)C(=O)N(C)C1C(=O)N(C)C1(CC1C)C(=O)OCC(NC(=O)c1nc3ccccc3cc1O)C(=O)NC(C)C(=O)N2C]

| | | | | |
|---|---|---|---|---|
| SW-163E/ | 260794-24- | Cancer and | not | 0.3 P388 |
| Streptomyces sp SNA | 9: 260794- | Antibacterial/ | reported | 0.2 A2780 |
| 15896/ | 25-0/ | low toxicity (mice | | 0.4 KB |
| SW-163E | SW-163C; | ip) | | 1.6 colon |
| | SW-163A; | | | 1.3 HL-60 |
| | SW-163B | | | |

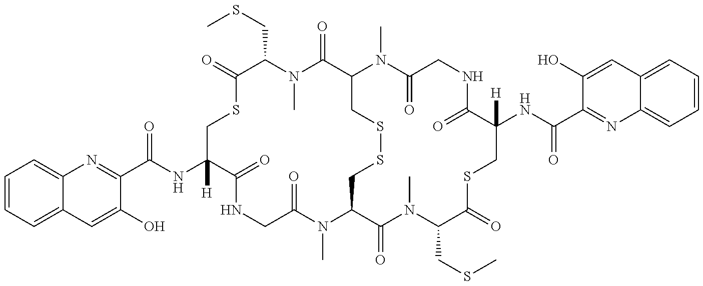 [SMILES: CSC[C@H]1C(=O)SC[C@@H](NC(=O)c2nc3ccccc3cc2O)C(=O)NCC(=O)N(C)[C@H]2CSSCC(C(=O)N1C)N(C)C(=O)CNC(=O)[C@H](NC(=O)c1nc3ccccc3cc1O)CSC(=O)[C@H](CSC)N(C)C2=O]

| | | | | |
|---|---|---|---|---|
| Thiocaraline/ | 173046-02- | Breast Cancer; | DNA | lung, colon, CNS |
| Micromonospora | 1 | Melanoma; Non- | Polymerase | melanoma |
| marina | | small lung cancer/ | alpha | |
| (actinomycete) | | not reported | inhibitor | |
| | | | (blocks | |
| | | | cell | |
| | | | progression | |
| | | | from GI | |
| | | | to S) | |

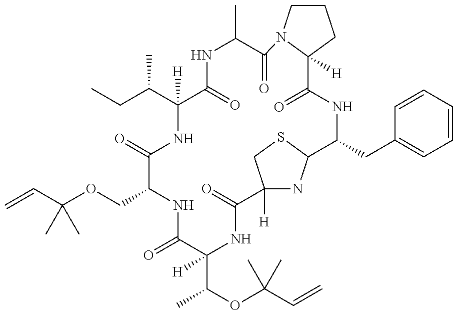 [SMILES: C=CC(C)(C)OC[C@H]1NC(=O)[C@H]([C@@H](C)OC(C)(C)C=C)NC(=O)C2CSC(N2)[C@@H](Cc2ccccc2)NC(=O)[C@@H]2CCCN2C(=O)C(C)NC(=O)[C@H]([C@@H](C)CC)NC1=O]

| | | | | |
|---|---|---|---|---|
| Trunkamide A¹/ | 181758-83- | Cancer/ | not | cell culture (IC50 in |
| Lissoclinum sp | 8 | not reported | reported | micrograms/mL); |
| (aascidian) | | | | 0.5 P388; |
| | | | | 0.5 A549; |
| | | | | 0.5 HT-29; |
| | | | | 1.0 MEL-28 |

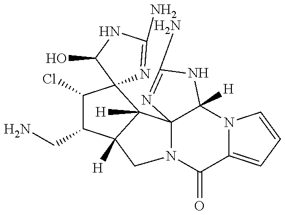

| | | | | |
|---|---|---|---|---|
| Palauamine²/ | 18717-58- | Lung cancer/ | not | cell culture (IC50 in |
| Stylotella agminata | 2 | LD50 (i.p. in mice) | reported | micrograms/mL); |
| (sponge) | | is 13 mg/Kg | | 0.1 P388 |
| | | | | 0.2 A549 (lung) |
| | | | | 2 HT-29 (colon) |
| | | | | 10 KB |

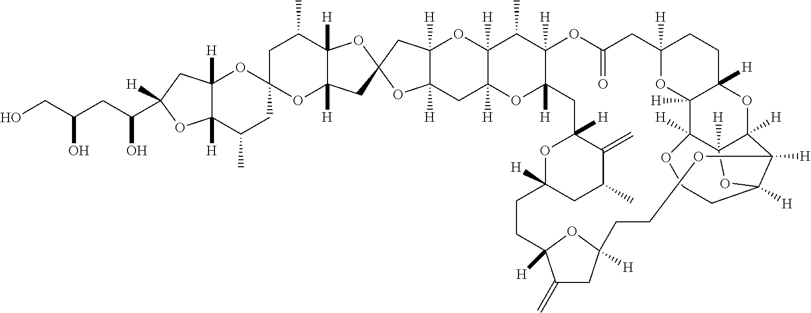 [SMILES: C=C1C[C@@H]2CCO[C@H]3[C@@H]4O[C@H]5CC[C@H](CC(=O)O[C@@H]6[C@@H](C)[C@@H]7O[C@@H]8C[C@]9(C[C@@H]%10O[C@]%11(C[C@H](C)[C@@H]%12O[C@H]([C@@H](O)C[C@@H](O)CO)C[C@@H]%12O%11)C[C@H](C)[C@@H]%10O9)O[C@@H]8C[C@@H]7O[C@H]6C[C@H]6O[C@@H](CC[C@@H]1O2)C[C@@H](C)C6=C)O[C@@H]5[C@@H]1OCC[C@H]3O[C@@H]14]

| | | | | |
|---|---|---|---|---|
| Halichondrin B/ | 103614-76- | cancer/ | antitubulin; | NCI tumor panel; |
| Halichondria Okadai, | 2/ | myelotoxicity dose | cell | GI(50) from 50 nM |
| Axinell Carteri and | isohomohal | limiting (dogs, rats) | cycle | to 0.1 nM; |
| Phankell carteri | ichondrin B | | inhibitor | LC50's from 40 μM |
| (sponges)/ | | | (inhibits | to 0.1 nM (many |
| NSC-609385 | | | GTP | 0.1 to 25 nM) |
| | | | binding to | |
| | | | tubulin) | |

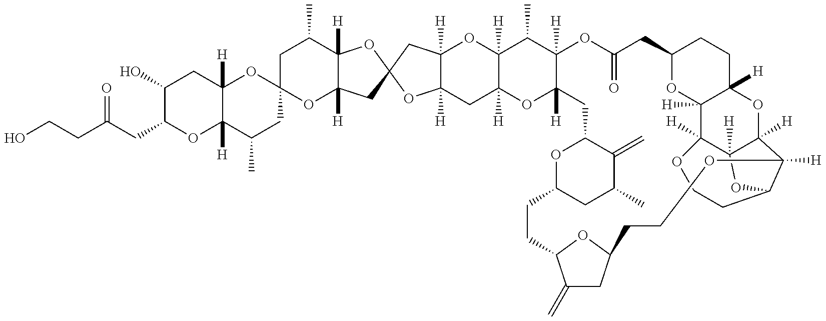 [SMILES: C=C1C[C@@H]2CCO[C@H]3[C@@H]4O[C@H]5CC[C@H](CC(=O)O[C@@H]6[C@@H](C)[C@@H]7O[C@@H]8C[C@]9(C[C@@H]%10O[C@]%11(C[C@H](C)[C@@H]%12O[C@H](CC(=O)CCO)[C@H](O)C[C@@H]%12O%11)C[C@H](C)[C@@H]%10O9)O[C@@H]8C[C@@H]7O[C@H]6C[C@H]6O[C@@H](CC[C@@H]1O2)C[C@@H](C)C6=C)O[C@@H]5[C@@H]1OCC[C@H]3O[C@@H]14]

| | | | | |
|---|---|---|---|---|
| Isohomo- | 157078-48- | melanoma, lung, | antitubulin; | IC50's in 0.1 nM |
| halichondrin B/ | 3/ | CNS, colon, ovary/ | cell cycle | range (NCI tumor |
| Halichondria Okadai, | halichondrin | not reported | inhibitor | panel) |
| Axinell Carteri and | B | | (inhibits | |
| Phankell carteri | | | GTP | |
| (sponges)/ | | | binding to | |
| NSC-650467 | | | tubulin) | |

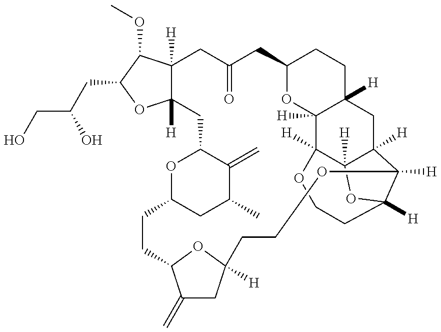 [SMILES: C=C1C[C@@H]2CCO[C@H]3[C@@H]4C[C@H]5CC[C@H](CC(=O)C[C@@H]6[C@@H](OC)[C@@H](C[C@H](O)CO)O[C@H]6C[C@H]6O[C@@H](CC[C@@H]1O2)C[C@@H](C)C6=C)O[C@@H]5[C@@H]1OCC[C@@H]3O[C@H]41]

| | | | | |
|---|---|---|---|---|
| Halichondrin B | 253128-15- | solid tumors/ | tubulin | cell culture (not |
| analogs/ | 3/ | not reported | binding | reported); |
| semi-synthetic | ER-076349; | | agent; | animal models |
| starting from | ER-086526; | | disruption | active (tumor |
| Halichondria Okadai, | B-1793; | | of mitotic | regression |
| Axlinell Carteri and | E-7389 | | spindles | observed) in |
| Phankell carteri | | | | lymphoma, colon |
| (sponges)/ | | | | (multi-drug |
| ER-076349; ER- | | | | resistant). |
| 086526; B-1793; E- | | | | |
| 7389 | | | | |

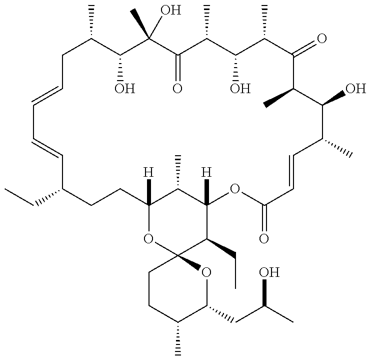 [SMILES: CC[C@@H]1/C=C/C=C/C[C@H](C)[C@@H](O)[C@](C)(O)C(=O)[C@H](C)[C@@H](O)[C@H](C)C(=O)[C@H](C)[C@@H](O)[C@H](C)/C=C/C(=O)O[C@H]2[C@@H](C)[C@@H](CC1)O[C@@]1(CC[C@@H](C)[C@@H](C[C@H](C)O)O1)[C@@H]2CC]

| | | | | |
|---|---|---|---|---|
| NK-1301129/ | 132707-68- | antifungal and | not | 25 ng/mL colon |
| Streptomyces | 7 | anticancer/ | reported | 8.5 ng/mL lung |
| bottropensis/ | | not reported | | |
| NK-130119 | | | | |

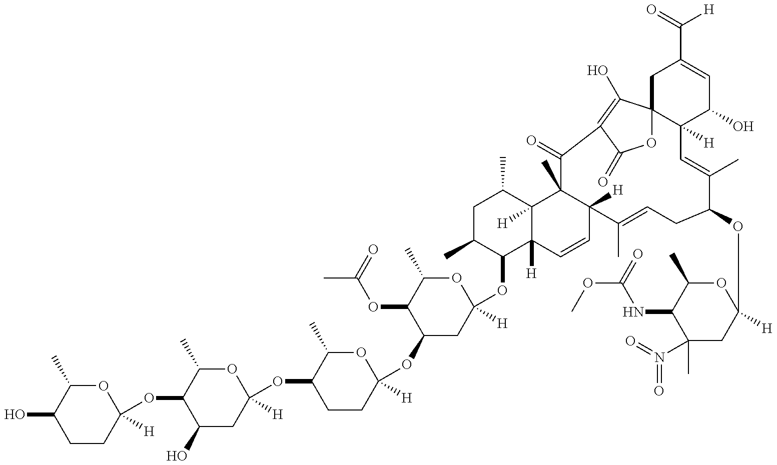

| | | | | |
|---|---|---|---|---|
| Tetrocracin A/ | 73666-84- | cancer/ | inhibits the | not reported |
| not reported/ | 9/ | not reported | anti- | |
| KF-67544 | analogs are | | apoptotic | |
| | reported | | functino of | |
| | | | Bcl2 | |

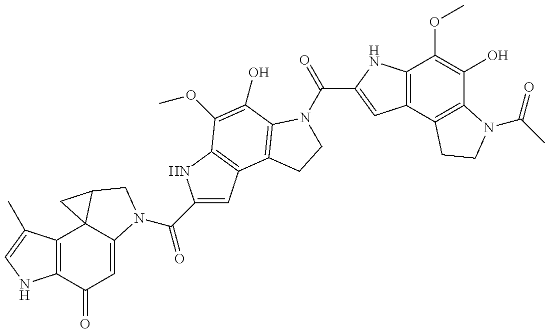 [SMILES: COc1c(O)c2c(c3cc(C(=O)N4CCc5c4c(O)c(OC)c4[nH]c(C(=O)N6CC7CC78C6=CC(=O)c6[nH]cc(C)c68)cc54)[nH]c13)CCN2C(C)=O]

| | | | | |
|---|---|---|---|---|
| Gilvusmycin/ | 195052-09- | cancer/ | not | IC50's in ng/mL: |
| Streptomyces QM16 | 6 | not reported | reported | 0.08 P388 |
| | | | | 0.86 K562 (CML) |
| | | | | 0.72 A431 (EC) |
| | | | | 0.75 MKN28 (GI); |
| | | | | (for all < 1 nM) |

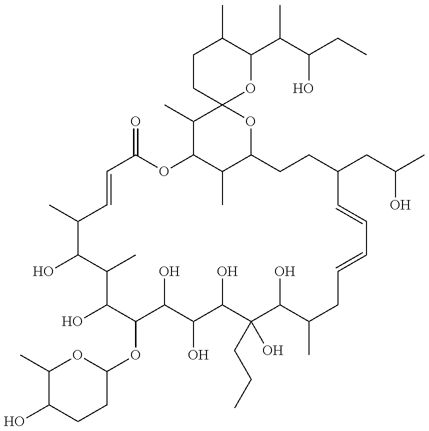 [SMILES: CCCC1(O)C(O)C(C)C/C=C/C=C/C(CC(C)O)CCC2OC3(CCC(C)C(C(C)C(O)CC)O3)C(C)C(OC(=O)/C=C/C(C)C(O)C(C)C(O)C(OC3CCC(O)C(C)O3)C(O)C(O)C1O)C2C]

| | | | | |
|---|---|---|---|---|
| IB-96212/ | 220858-11- | Cancer and | not | IC50's in ng/mL: |
| marine actinomycete/ | 7/ | Antibacterial/ | reported | 0.1 P388 |
| IC-96212 | IB-96212; | not reported | | |
| | IB-98214; | | | |
| | IB-97227 | | | |

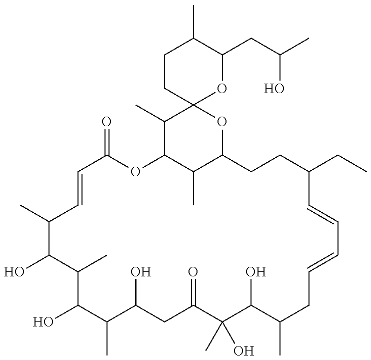 [SMILES: CCC1/C=C/C=C/CC(C)C(O)C(C)(O)C(=O)CC(O)C(C)C(O)C(C)C(O)C(C)/C=C/C(=O)OC2C(C)C(CC1)OC1(CCC(C)C(CC(C)O)O1)C2C]

| | | | | |
|---|---|---|---|---|
| BE-56384³/ | 207570-04- | cancer/ | not | IC50's in ng/mL: |
| Streptomyces Sp./ | 5 | not reported | reported | 0.1 P388 |
| BE-56384 | | | | 0.29 colon 26 |
| | | | | 34 DLD-1 |
| | | | | 0.12 PC-13 |
| | | | | 0.12 MKM-45 |

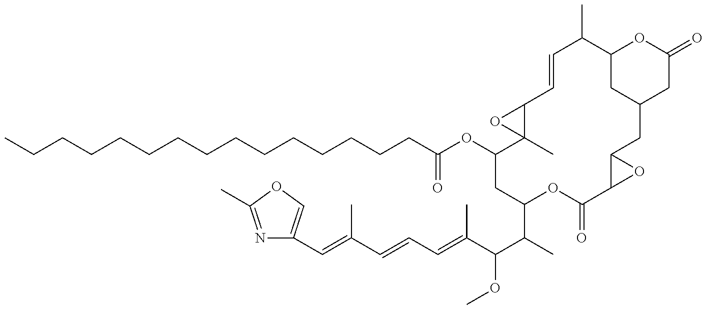 [SMILES: CCCCCCCCCCCCCCCC(=O)OC1CC(C(C)C(OC)/C(C)=C/C=C/C(C)=C/c2coc(C)n2)OC(=O)C2OC2CC2CC(=O)OC(C2)C(C)/C=C/C2OC21C]

| | | | | |
|---|---|---|---|---|
| Palmitoylrhizoxin/ | 135819-69- | cancer/ | tubulin | not reported |
| semi-synthetic; | 1/ | binds LDL; less | binding | |
| Rhizopus chinensis | Analog of | cytotoxic than | agent (cell | |
| | rhizoxin | rhizoxin | cycle | |
| | | | inhibitor) | |

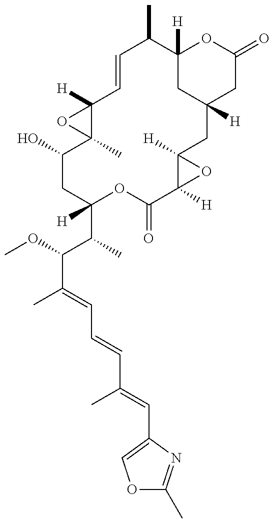 [SMILES: CO[C@@H](/C(C)=C/C=C/C(C)=C/c1coc(C)n1)[C@@H](C)[C@@H]1C[C@H](O)[C@@]2(C)O[C@@H]2/C=C/[C@@H](C)[C@H]2C[C@H](CC(=O)O2)C[C@H]2O[C@H]2C(=O)O1]

| | | | | |
|---|---|---|---|---|
| Rhizoxin/ | 95917-95- | melanoma, lung, | tubulin | NCI tumor panel |
| Rhizopus chinensis/ | 6; 90996- | CNS, colon, ovary, | binding | (NSC 332598); |
| WF-1360; NSC- | 54-6 | renal, breast, head | agent (cell | log GI50's: |
| 332598; FR-900216 | | and neck/ | cycle | 50 nM to 50 fM; |
| | | Rapid Drug | inhibitor) | log LC50's: |
| | | clearance; High | | 50 μM to 0.5 nM |
| | | AUC correlates with | | (several cell lines at |
| | | high toxicity | | 50 fM). |

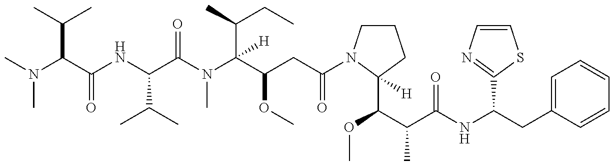 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C)C(C)C]

| | | | | |
|---|---|---|---|---|
| Dolastatin-10/ | 110417-88- | prostate, melanoma, | tubulin | NCI tumor panel |
| Dolabella auricularia | 4/ | leukemia/ | binding | (60 cell line; |
| (sea hare)/ | other | myelotoxicity (at | (tubulin | GI50); |
| NSC-376128 | Dolistatins | greater than 0.3 pM) | aggregation) | 25 nM to 1 pM |
| | (ie. 15) and | | | (most < 1 nM) |
| | analogs | | | (three cell lines |
| | | | | μM) |

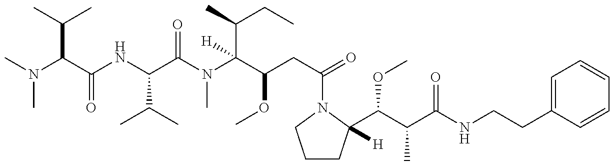 [SMILES: CC[C@H](C)[C@@H]([C@@H](CC(=O)N1CCC[C@H]1[C@H](OC)[C@@H](C)C(=O)NCCc1ccccc1)OC)N(C)C(=O)[C@@H](NC(=O)[C@H](C(C)C)N(C)C)C(C)C]

| | | | | |
|---|---|---|---|---|
| soblidotin/ | 149606-27- | cancer (pancreas, | tubulin | cell culture: colon, |
| synthetic/ | 9/ | esophageal colon, | binding | melanoma, M5076 |
| TZT-1027; auristatin | analogs | breast, lung, etc)/ | agent | tumors, P388 with |
| PE | prepared | MTD was 1.8 | 75-85% inhibition | |
| | | mg/Kg (IV); | (dose not reported) | |
| | | toxicity not reported | | |

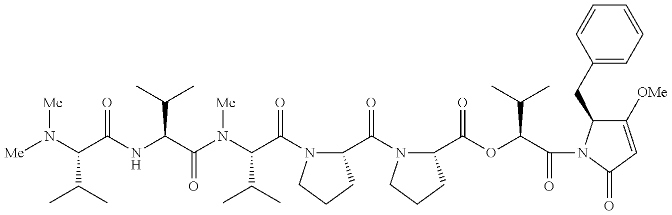 [SMILES: COC1=CC(=O)N(C(=O)[C@@H](OC(=O)[C@@H]2CCCN2C(=O)[C@@H]2CCCN2C(=O)[C@H](C(C)C)N(C)C(=O)[C@@H](NC(=O)[C@H](C(C)C)N(C)C)C(C)C)C(C)C)[C@H]1Cc1ccccc1]

| | | | | |
|---|---|---|---|---|
| Dolastatin-15/ | not | cancer/ | Tubulin | NCI tumor panel |
| Dolabella auricularia | reported/ | not reported | binding | (60 cell line; |
| (sea hare) | other | | (tubuline | GI50); 25 nM to 39 |
| | Dolistatins | | aggregation) | pM (most < 1 nM) |
| | (ie. 15) and | | | (one cell line 2.5 |
| | analogs | | | μM); most active in |
| | | | | breast |

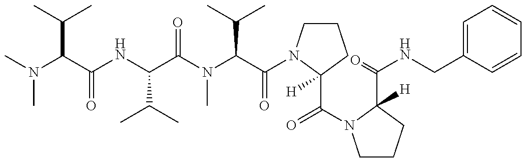 [SMILES: CC(C)[C@H](NC(=O)[C@H](C(C)C)N(C)C)C(=O)N(C)[C@H](C(=O)N1CCC[C@H]1C(=O)N1CCC[C@H]1C(=O)NCc1ccccc1)C(C)C]

| | | | | |
|---|---|---|---|---|
| Cemadotin⁴/ | 1159776- | melanoma/ | tubulin | NCI tumor panel |
| Synthetic; Parent | 69-9/ | hypertension, | binding | (NCS D-669356); |
| Dolastatin-15 was | many | myocardial ischemia | (tubulin | active in breast, |
| isolated from | analogs | and | aggregation) | ovary, endometrial, |
| Dolabella auricularia | | myelosuppresion | | sarcomas and drug |
| (sea hare)/ | | were dose-limiting | | resistant cell lines |
| LU-103793; NSC D- | | toxicities. | | Data not public. |
| 669356 | | | | |

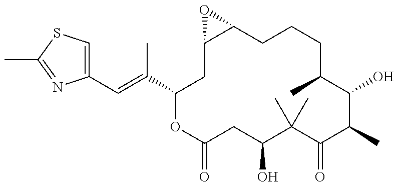 [SMILES: C/C(=C\c1csc(C)n1)[C@@H]1C[C@@H]2O[C@@H]2CCC[C@H](C)[C@H](O)[C@@H](C)C(=O)C(C)(C)[C@@H](O)CC(=O)O1]

| | | | | |
|---|---|---|---|---|
| Epothilone A/ | not | cancer/ | tubulin | IC50's of; |
| Synthetic or isolated | reported/ | not reported | binding | 1.5 nM MCF-7 |
| from Sorangium | many | | (tubulin | (breast) |
| cellulosum | analogs | | polymeriza- | 27.1 nM MCF- |
| (myxococcales) strain | | | tion) | 7/ADR |
| So ce90) | | | | 2.1 nM KB-31 |
| | | | | (melanoma) |
| | | | | 3.2 nM HCT-116 |

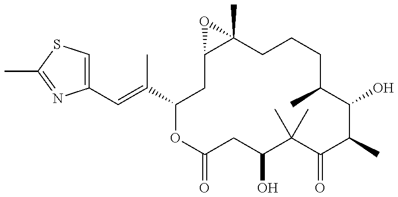

| | | | | |
|---|---|---|---|---|
| Epothione B/ | 152044054- | Solid tumors (breast, | tubulin | IC50's of; |
| Synthetic or isolated | 7/ | ovarian, etc)/ | binding | 0.18 nM MCF-7 |
| from Sorangium | many | well tolerated; t1/2 | (tubulin | breast) |
| cellulosum | analogs | of 2.5 hrs; partial | polymeriza- | 2.92 nM MCF- |
| (myxococcales) strain | | responses (phase I); | tion) | 7/ADR |
| So ce90)/ | | diarrhea major side | | 0.19 nM KB-31 |
| EPO-906 | | effect. | | (melanoma) |
| | | | | 0.42 nM HCT-116; |
| | | | | broad activity |
| | | | | reported |

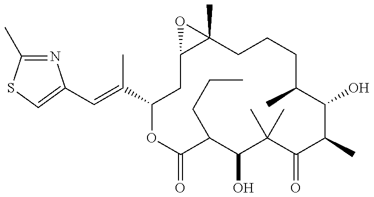

| | | | | |
|---|---|---|---|---|
| Epothilone Analog/ | not reported/ | cancer/ | tubulin | IC50's of 0.30 to |
| Synthetic or semi- | hundreds of | not reported | binding | 1.80 nM in various |
| synthetic; Original | analogs | | (tubulin | tumor cell lines; |
| lead, Epothilone A, | | | polymeriza- | active in drug |
| isolated from | | | tion) | resistant cell lines |
| Sorangium cellulosum | | | | |
| (myxococcales) strain | | | | |
| So ce90)/ | | | | |
| ZK-EPO | | | | |

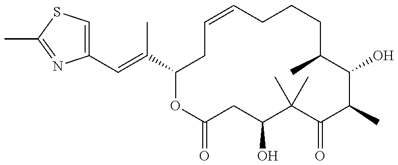

| | | | | |
|---|---|---|---|---|
| Epothilone D/ | 189452-10- | Solid tumors (breast, | tubulin | NCI tumor panel |
| Epothilone D, isolated | 9/ | ovarian, etc)/ | binding | (NSC-703147; |
| from Sorangium | many | emesis and anemia; | (tubulin | IC50); |
| cellulosum | analogs | t1/2 of 5-10 hrs. | polymeriza- | 0.19 nM KB-31 |
| (myxococcales) strain | | | tion) | (melanoma) |
| So ce90)/ | | | | 0.42 nM HCT-116; |
| KOS-862 | | | | broad activity |
| | | | | reported |

Structure Not Identified

| | | | | |
|---|---|---|---|---|
| Epothilone D analog⁵/ | 189453-10- | Solid tumors; | tubulin | not reported |
| Synthetic or semi- | 9/ | not reported | binding | |
| synthetic; Original | hundreds of | | (tubulin | |
| lead, Epothilone D, | analogs | | polymeriza- | |
| isolated from | | | tion) | |
| Sorangium cellulosum | | | | |
| (myxococcales) strain | | | | |
| So ce90)/ | | | | |
| IOS-166-24 | | | | |

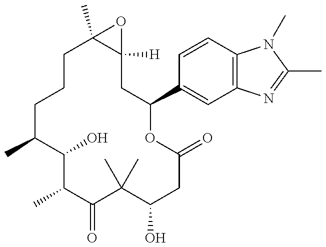

| | | | | |
|---|---|---|---|---|
| Epothilone Analog/ | not | cancer; | tubulin | not reported |
| Synthetic; Original | reported/ | not reported | binding | |
| lead, Epothilone A, | hundreds of | | (tubulin | |
| isolated from | analogs | | polymeriza- | |
| Sorangium cellulosum | | | tion) | |
| (myxococcales) strain | | | | |
| So ce90)/ | | | | |
| CGP-85715 | | | | |

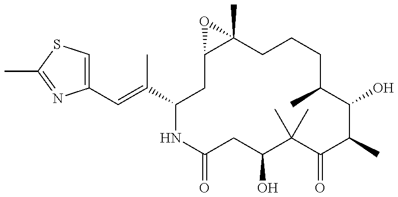

| | | | | |
|---|---|---|---|---|
| Epothilone Analog/ | 219989-84- | non-small cell Lung, | tubulin | NCI tumor Panel |
| Synthetic or semi- | 1/ | breast, stomach | binding | (NSC-710428 & |
| synthetic; Original | hundreds of | tumor (objective | (Tubulin | NSC-710468); 8- |
| lead, Epothilone B, | analogs | responses in breast | polymeriza- | 32 nM (NCI data |
| isolated from | | ovarian and lung)/ | tion) | not available) |
| Sorangium cellulosum | | sever toxicity | | |
| (myxococcales) strain | | (fatigue, anorexia, | | |
| So ce90)/ | | nauseas, vomiting, | | |
| BMS-247550 | | neuropathy myalgia) | | |

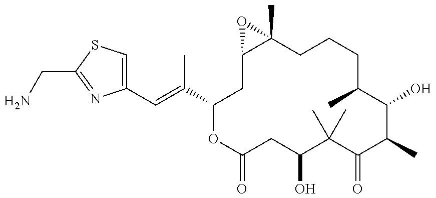 [SMILES: C/C(=C\c1csc(CN)n1)[C@@H]1C[C@@H]2O[C@]2(C)CCC[C@H](C)[C@H](O)[C@@H](C)C(=O)C(C)(C)[C@@H](O)CC(=O)O1]

| | | | | |
|---|---|---|---|---|
| Epothilone Analog/ | not | advanced cancers/ | tubulin | broad activity with |
| Synthetic or semi- | reported/ | adverse events | binding | IC50's of 0.7 to 10 |
| synthetic; Original | hundreds of | (diarrhea, nausea, | (tubulin | nM |
| lead, Epothilone B, | analogs | vomiting, fatigue, | polymeriza- | |
| isolated from | | neutropenia); t1/2 of | tion) | |
| Sorangium cellulosum | | 3.5 hrs; improved | | |
| (myxococcales) strain | | water solubility to | | |
| So ce90)/ | | BMS2 247550. | | |
| BMS-310705 | | | | |

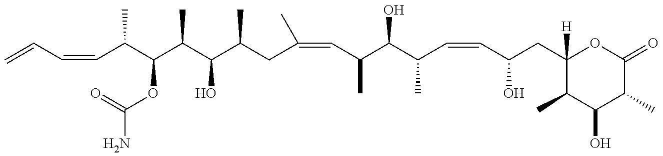 [SMILES: C=C/C=C\[C@H](C)[C@H](OC(N)=O)[C@@H](C)[C@H](O)[C@@H](C)C/C(C)=C\[C@H](C)[C@@H](O)[C@@H](C)/C=C\[C@@H](O)C[C@@H]1OC(=O)[C@H](C)[C@@H](O)[C@H]1C]

| | | | | |
|---|---|---|---|---|
| Discodermolide/ | 127943-53- | solid tumors/ | tubulin | Broad activity |
| synthetic; orginally | 7/ | not reported; 100- | stabilizing | (A549-nsclung, |
| isolated from | analogs less | fold increase in | agent | prostate, P388, |
| Discodermia | potent | water solubility over | (similar to | ovarian with IC50's |
| dissoluta (deep water | | taxol | taxol) | about 10 mM) |
| sponge); rare | | | | including multi- |
| compound (7 mg per | | | | drug resistant cell |
| 0.5 Kg sponge/ | | | | lines; |
| XAA-296 | | | | |

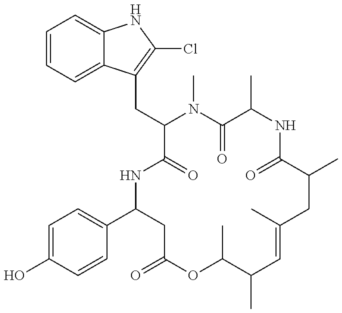 [SMILES: C/C1=C\C(C)C(C)OC(=O)CC(c2ccc(O)cc2)NC(=O)C(Cc2c(Cl)[nH]c3ccccc23)N(C)C(=O)C(C)NC(=O)C(C)C1]

| | | | | |
|---|---|---|---|---|
| Chondramide D/ | 172430-63- | cancer/ | tubulin | 5 nM A-549 |
| not reported | 6 | not reported | binding | (epidermoid |
| | | | agent; | carcinoma) |
| | | | actin | 15 nM A-498 |
| | | | polymeriza- | (kidney) |
| | | | tion | 14 nM A549 (lung) |
| | | | inhibitor | 5 nM SK-OV-3 |
| | | | | (ovary) |
| | | | | 3 nM U-937 |
| | | | | (lymphoma) |

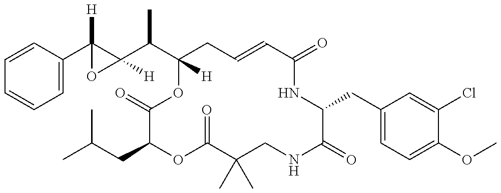 [SMILES: COc1ccc(C[C@H]2NC(=O)/C=C/C[C@@H]([C@H](C)[C@H]3O[C@@H]3c3ccccc3)OC(=O)[C@H](CC(C)C)OC(=O)C(C)(C)CNC2=O)cc1Cl]

| | | | | |
|---|---|---|---|---|
| Cryptophycin | 204990-60- | solid tumors, colon | tubulin | broad activity |
| analogs (including | 3 and | cancer/ | polymeriza- | (lung, breast, colon, |
| 52, 55 and others)⁶/ | 186256-67- | Phase II studies | tion | leukemia) with |
| Nostoc sp GSV 224 | 7/ | halted because of | inhibitor | IC50's of 2 to 40 |
| (blue-green algae) | many | severe toxicity with | | pM; active against |
| isolated Cryptophycin | potent | one death resulting | | multi-drug |
| 1./ | analogs | from drug; | | resistance cell lines |
| LY-355703; Ly- | prepared at | | | (resistant to MDR |
| 355702; NSC- | Lilly | | | pump). NCI tumor |
| 66762 | | | | panel, GI50's from |
| | | | | 100 nM to 10 pM; |
| | | | | LC50's from 100 |
| | | | | nM to 25 pM. |

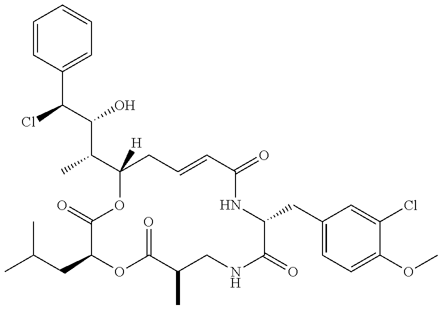 [SMILES: COc1ccc(C[C@H]2NC(=O)/C=C/C[C@@H]([C@H](C)[C@@H](O)[C@@H](Cl)c3ccccc3)OC(=O)[C@H](CC(C)C)OC(=O)[C@H](C)CNC2=O)cc1Cl]

| | | | | |
|---|---|---|---|---|
| Cryptophycin 8/ | 168482-36- | solid tumors/ | tubulin | broad spectrum |
| semi-synthetic; | 8; 168482- | not reported | polymeriza- | anticancer activity |
| starting material from | 40-4; | | tion | (cell culture) |
| Nostoc sp. | 18665-94- | | inhibitor | including multi- |
| | 1; 124689- | | | drug resistant |
| | 65-2; | | | tumors |
| | 125546-14- | | | |
| | 7/ | | | |
| | cryptophycin | | | |
| | 5, 15 and | | | |
| | 35 | | | |

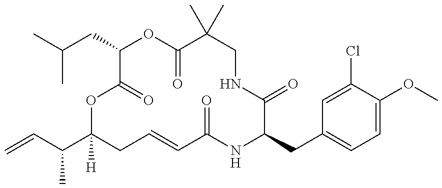 [SMILES: C=C[C@@H](C)[C@@H]1C/C=C/C(=O)N[C@H](Cc2ccc(OC)c(Cl)c2)C(=O)NCC(C)(C)C(=O)O[C@@H](CC(C)C)C(=O)O1]

| | | | | |
|---|---|---|---|---|
| Cryptophycin | 219660-54- | solid tumors/ | topoisomer- | not reported |
| analogs⁷/ | 5/ | not reported | ase | |
| synthetic; semi- | LY-404292 | | inhibitors | |
| synthetic, starting | | | | |
| material from Nostoc | | | | |
| sp./ | | | | |
| LY-404291 | | | | |

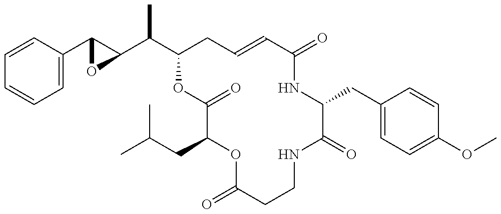 [SMILES: COc1ccc(C[C@H]2NC(=O)/C=C/C[C@@H]([C@H](C)[C@H]3O[C@@H]3c3ccccc3)OC(=O)[C@H](CC(C)C)OC(=O)CCNC2=O)cc1]

| | | | | |
|---|---|---|---|---|
| Arenastatin A | not | cancer/ | inhibits | 8.7 nM (5 pg/mL) |
| analogs⁸/ | reported/ | not reported | tubulin | KB |
| Dysidea arenaria | analogs | | polymeriza- | (nasopharyngeal); |
| (marine sponge)/ | prepared | | tion | NCI tumor panel |
| Cryptophycin B; | | | | (GI50's); 100 pM |
| NSC-670038 | | | | to 3 pM |

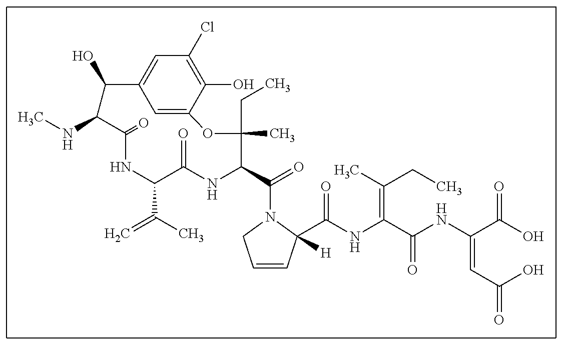 [SMILES: C=C(C)[C@@H]1NC(=O)[C@@H](NC)[C@@H](O)c2cc(Cl)c(O)c(c2)O[C@](C)(CC)[C@@H](C(=O)N2CC=C[C@H]2C(=O)N/C(C(=O)N/C(=C/C(=O)O)C(=O)O)=C(\C)CC)NC1=O]

| | | | | |
|---|---|---|---|---|
| Phomopsin A/ | not reported | Liver cancer (not as | tubulin | potent anticancer |
| Diaporte toxicus or | | potent in other | binding | activity especially |
| Phomopsin | | cancers)/ | agent | against liver cancer |
| leptostromiformis | | not reported | | |
| (fungi) | | | | |

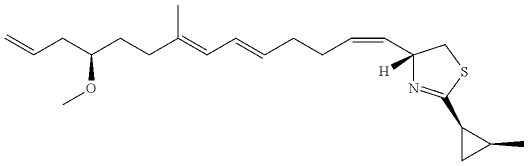 [SMILES: C=CC[C@@H](CC/C(C)=C/C=C/CC/C=C\[C@@H]1CSC([C@@H]2C[C@@H]2C)=N1)OC]

| | | | | |
|---|---|---|---|---|
| Curacin A and | 155233-30- | Cancer/ | Tubulin | broad activity |
| analogs/ | 0/ | not reported | binding | (cancer cell lines); |
| Lyngbya majuscula | analogs | | agent | 1-29 nM |
| (blue green | have been | | | |
| cyanobacterium) | prepared | | | |

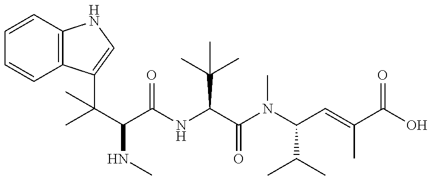 [SMILES: CN[C@H](C(=O)N[C@H](C(=O)N(C)[C@H](/C=C(\C)C(=O)O)C(C)C)C(C)(C)C)C(C)(C)c1c[nH]c2ccccc12]

| | | | | |
|---|---|---|---|---|
| Hemiasterlins A & B | not | Cancer/ | Antimitotic | broad activity: |
| and analogs⁹/ | reported/ | not reported | agent | 0.3-3 nM MCF7 |
| Cymbastela sp. | criamide A | | (tubulin | (breast); |
| | & B; | | binding | 0.4 ng/mL P388 |
| | geodiamiolid- | | agent) | |
| | G | | | |

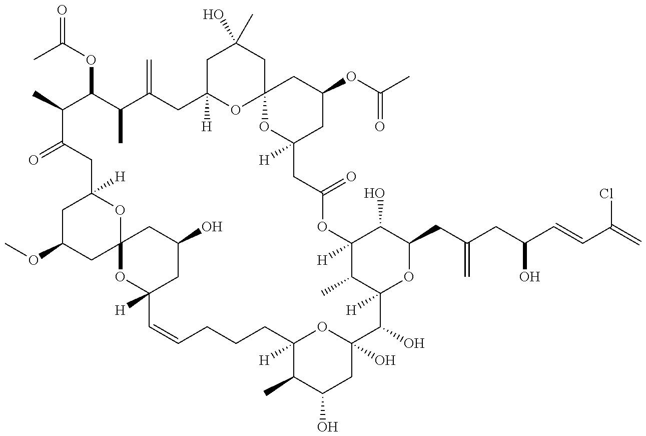

| | | | | |
|---|---|---|---|---|
| Spongistatins (1-9)¹⁰/ | 149715-96- | cancer/ | tubulin | Most potent |
| Spirastrell | 8; 158734- | not reported | binding | compounds ever |
| spinispirulifera (sea | 18-0; | | agent | tested in NCI panel |
| sponge) | 158681-42- | | | cell line (mean |
| | 6; 158080- | | | GI50's of 0.1 nM; |
| | 65-0; | | | Spongistatin-1 |
| | 150642-07- | | | GI50's of 0.025- |
| | 2; 153698- | | | 0.035 nM with |
| | 80-7; | | | extremely potent |
| | 153745-94- | | | activity against a |
| | 9; 150624- | | | subset of highly |
| | 44-5; | | | chemoresistant |
| | 158734-19- | | | tumor types |
| | 1/ | | | |
| | other | | | |
| | spongistatins | | | |

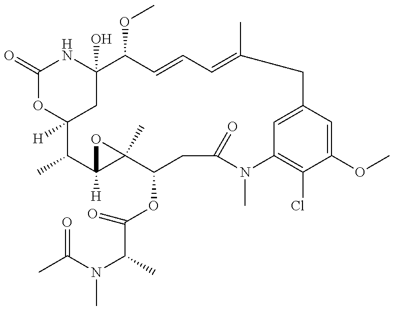

| | | | | |
|---|---|---|---|---|
| Maytansine/ | 35846-53- | cancer/ | tubulin | Broad Activity in |
| Maytenus sp./ | 8/ | severe toxicity | binding | NCI tumor panel |
| NSC-153858 | other | | agent | (NSC-153858; |
| | related | | (causes | NSC-153858); |
| | macrolides | | extensive | NCI tumor panel, |
| | | | diassembly | GI50's from 3 μM |
| | | | of the | to 0.1 pM; LC50's |
| | | | microtubule | from 250 μM to 10 |
| | | | and | pM. Two different |
| | | | totally | experiments gave |
| | | | prevents | very different |
| | | | tubulin | potencies. |
| | | | spiralizaiton) | |

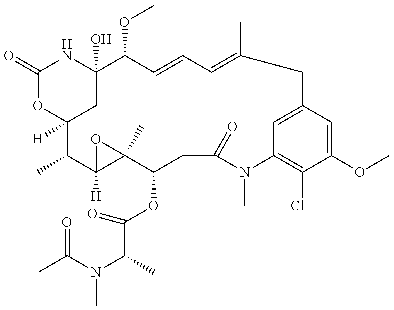 [SMILES: COc1cc2cc(c1Cl)N(C)C(=O)C[C@H](OC(=O)[C@H](C)N(C)C(C)=O)[C@]1(C)O[C@@H]1[C@H](C)[C@@H]1C[C@@](O)(NC(=O)O1)[C@H](OC)/C=C/C=C(\C)C2]

| | | | | |
|---|---|---|---|---|
| Maytansine- | not | breast, head and | EGFR | not reported |
| IgG(EGFR | reported/ | neck, Squamous cell | binding | |
| directed)- | other | carcoinoma/ | and | |
| conjugate¹¹/ | related | not reported | tubulin | |
| semi-synthetic; | macrolies | | binding | |
| starting material from | | | | |
| Maytenus sp. | | | | |

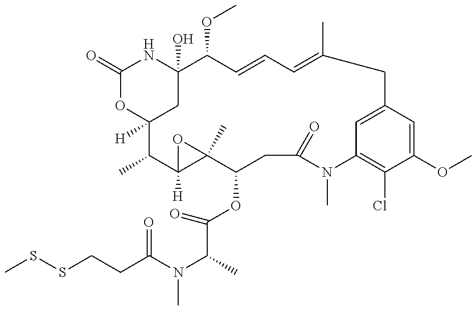

| | | | | |
|---|---|---|---|---|
| Maytansine- | not | Neuroendocrine, | CD56 | antigen-specific |
| IgG(CD56 antigen)- | reported/ | small-cell lung, | binding | cytotoxicity (cell |
| conjugate¹², 3.5 drug | other | carcinoma/ | and | culture; epidermal, |
| molecules per IgG/ | related | mild toxicity | tubulin | breast, renal |
| semi-synthetic; | macrolides | (fatigue, nausea, | binding | ovarian colon) with |
| starting material from | | headaches and mild | | IC50's of 10-40 |
| Maytenus sp./ | | peripheral | | pM; animal studies |
| huN901-DM1 | | neuropathy); no | | (miceSCLC tumor- |
| | | hematological | | alone and in |
| | | toxicity; MTD 60 | | combination with |
| | | mg/Kg, I.V., weekly | | taxol or cisplatin |
| | | for 4 weeks; only | | completely |
| | | stable disease | | eliminated tumors). |
| | | reported (humans) | | |

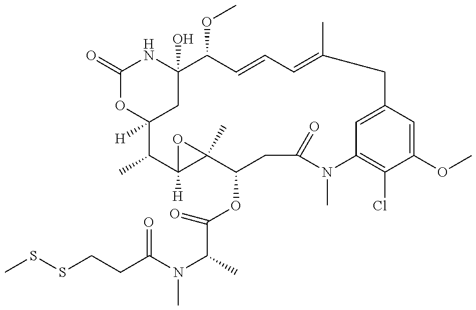

| | | | | |
|---|---|---|---|---|
| Maytansine- | not | non-small-cell lung, | CEA | antigen-specific |
| IgG(CEA antigen)- | reported/ | carcinoma pancreas, | binding | cytotoxicity (cell |
| conjugate¹³, 4 drug | other | lung, colon/ | and | culture; epidermal, |
| molecules per IgG/ | related | mild toxicity | tubulin | breast, renal |
| semi-synthetic; | macrolides | (fatigue, nausea, | binding | ovarian colon) with |
| starting material from | | headaches and mild | | IC50's of 10-40 |
| Maytenus sp./ | | peripheral | | pM; animal studies |
| C424-DM1 | | neuropathy); | | (mice: melanoma |
| | | pancreatic lipase | | [COLO-205]--alone |
| | | elevated; MTD 88 | | and in combination |
| | | mg/Kg, I.V., every | | with taxol or |
| | | 21 days; only stable | | cisplatin completely |
| | | disease reported | | eliminated tumors); |
| | | (humans); t1/2 was | | |
| | | 44 hr. | | |

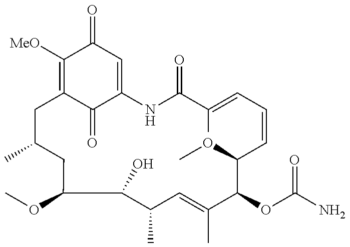 [SMILES: COC1=C2C[C@@H](C)C[C@H](OC)[C@H](O)[C@@H](C)/C=C(\C)[C@H](OC(N)=O)[C@@H](OC)/C=C\C=C(/C)C(=O)NC(=CC1=O)C2=O]

| | | | | |
|---|---|---|---|---|
| Geldanamycin/ | 30562-34- | cancer/ | binds Hsp | NCI tumor panel |
| Streptomyces | 6/ | not reported | 90 | (cell culture); 5.3 |
| hygroscopicus var. | natural | | chaperone | to 100 nM; most |
| Geldanus/ | derivatives | | and | active in colon, |
| NSC-212518; | | | inhibits | lung and leukemia. |
| Antibiotic U 29135; | | | function | NCI tumor panel, |
| NSC-122750 | | | | GI50's from 10 μM |
| | | | | to 0.1 nM; LC50's |
| | | | | from 100 μM to |
| | | | | 100 nM. Two |
| | | | | assays with very |
| | | | | different potencies. |

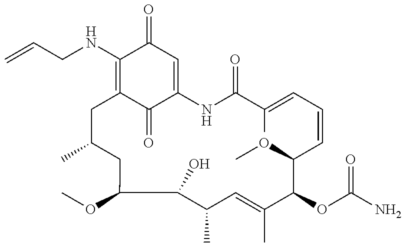 [SMILES: C=CCNC1=C2C[C@@H](C)C[C@H](OC)[C@H](O)[C@@H](C)/C=C(\C)[C@H](OC(N)=O)[C@@H](OC)/C=C\C=C(/C)C(=O)NC(=CC1=O)C2=O]

| | | | | |
|---|---|---|---|---|
| Geldanamycin | 745747-14- | solid tumors/ | binds Hsp | cell culture (not |
| Analog/ | 7/ | Dose limiting | 90 | reported); animal |
| semi-synthetic;/ | Kosan, NCI | toxicities (anemia, | chaperone | models active |
| CP-127374; 17- | and UK | anorexia, diarrhea, | and | (tumor regression |
| AAG; NSC-330507 | looking for | nausea nad | inhibits | observed) in breast, |
| | analogs | vomiting); t1/2 (i.v.) | function | ovary, melanoma, |
| | with longer | is about 90 min; no | | colon. |
| | t1/2 and | objective responses | | |
| | oral | measured at 88 | | |
| | activity; | mg/Kg (i.v. daily for | | |
| | analogs | 5 days, every 21 | | |
| | include: | days); | | |
| | NSC- | | | |
| | 255110; | | | |
| | 682300; | | | |
| | 683661; | | | |
| | 683663. | | | |

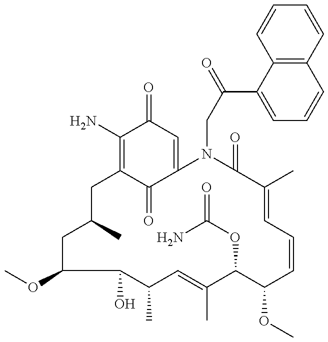 [SMILES: CO[C@H]1/C=C\C=C(/C)C(=O)N(CC(=O)c2cccc3ccccc23)C2=CC(=O)C(N)=C(C[C@@H](C)C[C@H](OC)[C@H](O)[C@@H](C)/C=C(\C)[C@@H]1OC(N)=O)C2=O]

| | | | | |
|---|---|---|---|---|
| Geldanamycin | not | solid tumors/ | binds Hsp | not reported |
| analog/ | reported/ | not reported | 90 | |
| semi-synthetic;/ | analogs | | chaperone | |
| CP-202567 | prepared | | and | |
| | | | inhbits | |
| | | | function | |

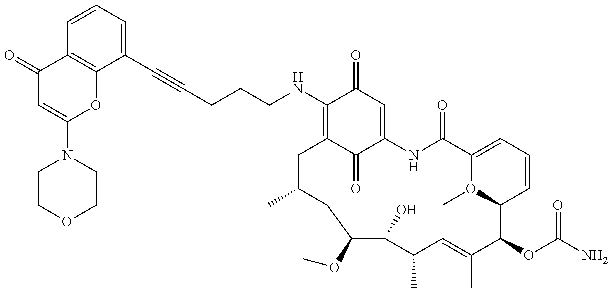

| | | | | |
|---|---|---|---|---|
| Geldanamycin | 345232-44- | breast/ | binds Hsp | cell culture (no |
| conjugates/ | 2/ | not reported | 90 | reported); animal |
| semi-synthetic;/ | analogs | | chaperone | models performed |
| LY-294002-GM; | prepared | | and | |
| PI3K-1-GM | | | inhibits | |
| | | | function; | |
| | | | binds and | |
| | | | inhibits PI- | |
| | | | 3 kinase | |

Structure Not Reported

| | | | | |
|---|---|---|---|---|
| Geldanamycin | not | breast, prostate/ | binds Hsp | not reported |
| Analog/ | reported/ | not reported | 90 | |
| not reported/ | analogs | | chaperone | |
| CNF-101 | prepared | | and | |
| | | | inhibits | |
| | | | function | |

Structure Not Reported

| | | | | |
|---|---|---|---|---|
| Geldanamycin- | not | prostate/ | binds Hsp | not reported; |
| testosterone | reported/ | not reported | 90 | conjugate has a 15- |
| conjugate/ | analogs | | chaperone | fold selective |
| semi-synthetic/ | prepared | | and | crytotoxicity for |
| GMT-1 | | | inhibits | androgen positive |
| | | | function | prostate cells |
| | | | and | |
| | | | testosterone | |
| | | | receptors | |
| | | | where it is | |
| | | | internalized | |

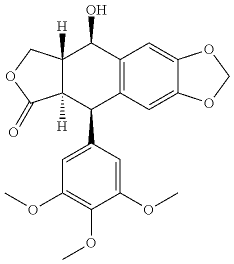 [SMILES: COc1cc([C@@H]2c3cc4c(cc3[C@H](O)[C@H]3COC(=O)[C@H]23)OCO4)cc(OC)c1OC]

| | | | | |
|---|---|---|---|---|
| Podophyllotoxin/ | 518-28-5/ | Verruca vulgaris, | tubulin | broad activity (cell |
| Podophyllum sp. | many | Condyloma/ | inhibitor | cutlure) with IC50's |
| | analogs | severe toxicity when | and | in μM range |
| | | given i.v. or s.c. | topoisomer- | |
| | | | ase | |
| | | | inhibitor | |

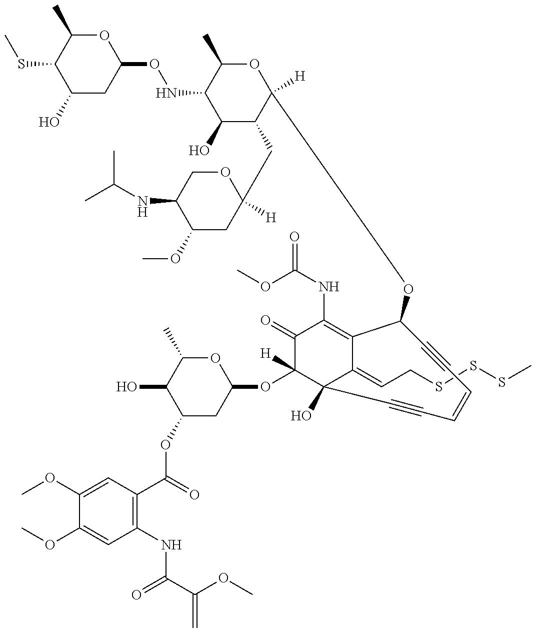

| | | | | |
|---|---|---|---|---|
| esperamicin-A1/ | 99674-26-7 | cancer/ | DNA | highly potent |
| not known/ | | not reported | cleaving | activity (cell |
| BBM-1675A1; | | (suspected severe | agent | culture); animal |
| BMY-28175; GGM- | | toxicity) | | models highly |
| 1675 | | | | potent with optimal |
| | | | | dose of 0.16 |
| | | | | micrograms/Kg |

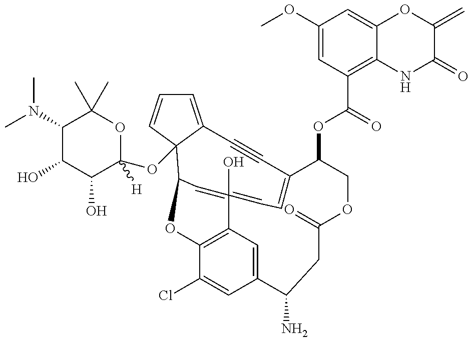

| | | | | |
|---|---|---|---|---|
| C-1027¹⁴/ | 120177-69- | cancer (examined | DNA | extremely potent |
| Streptomyces setonii | 7 | hepatoma, breast, | cleaving | (cell culture) IC50's |
| C-1027/ | | lung and leukemia/ | agent | in pM and fM; |
| C-1027 | | not reported | | conjugated to |
| | | | | antibodies the |
| | | | | potency remains the |
| | | | | same (ie. 5.5 to 42 |
| | | | | pM); |

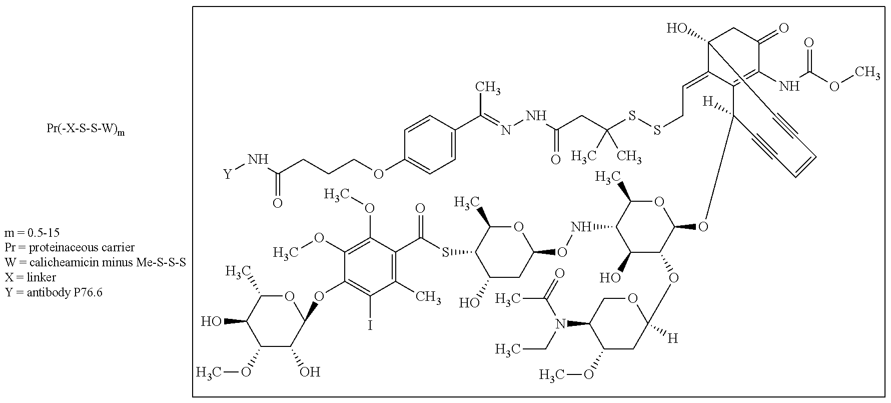

| | | | | |
|---|---|---|---|---|
| Calicheamicin- | 113440-58- | AML/ | DNA | Kills CD33+ cells |
| IgG(CD33 antigen)- | 7; 220578- | mild toxicity | cleaving | (HL-60, NOMO-1, |
| conjugate¹⁵/ | 59-6/ | | agent | and NKM-1) at 100 |
| semi-synthetic: | several | | | ng/mL; MDR cell |
| Micromonospora | reported in | | | lines are not |
| echinosporal | patents | | | effected by the |
| gemtuzumab | | | | drug. |
| ozogamicin; | | | | |
| mylotarg; WAY- | | | | |
| CMA-676; CMA- | | | | |
| 676; CDP-771 | | | | |

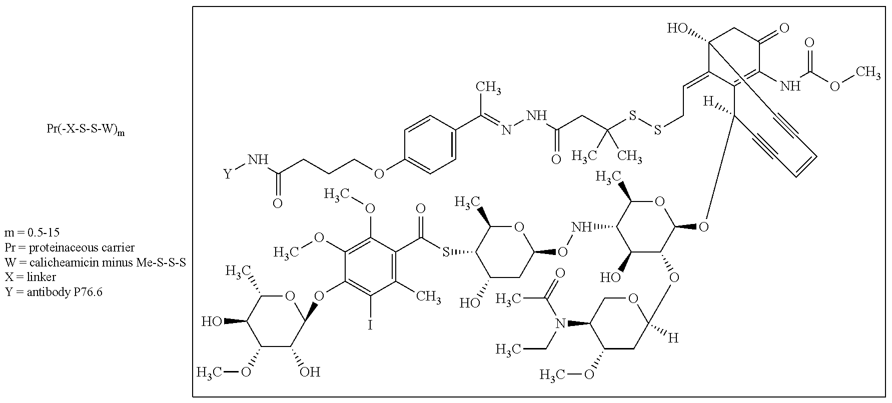

| | | | | |
|---|---|---|---|---|
| Calicheamicin-IgG- | 113440-58- | cancer/ | DNA | TBD |
| conjugates¹⁶/ | 7; 220578- | not reported | cleaving | |
| semi-synthetic: | 59-6 | | agent | |
| Micromonospora | | | | |
| echinospora | | | | |

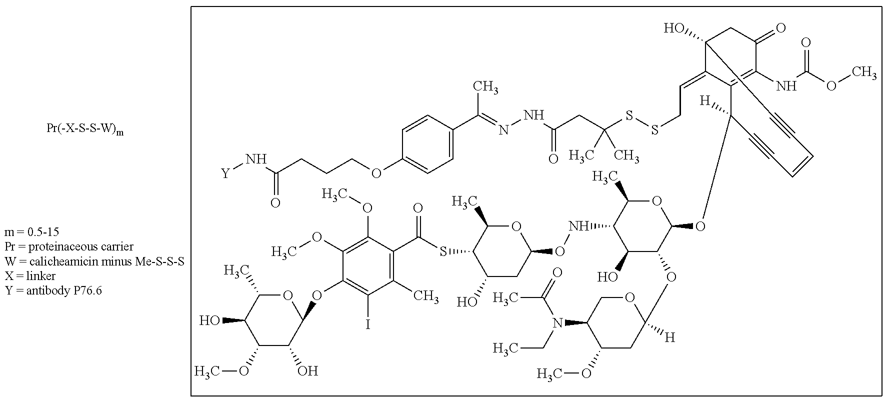

| | | | | |
|---|---|---|---|---|
| Calicheamicin- | not reported | cnacer/ | DNA | all human cancer; |
| IgG(OBA1 antigen) | | not reported | cleaving | data not reported |
| conjugate/ | | | agent | |
| semi-synthetic: | | | | |
| Micromonospora | | | | |
| echinosporal | | | | |
| OBA1-H8 | | | | |

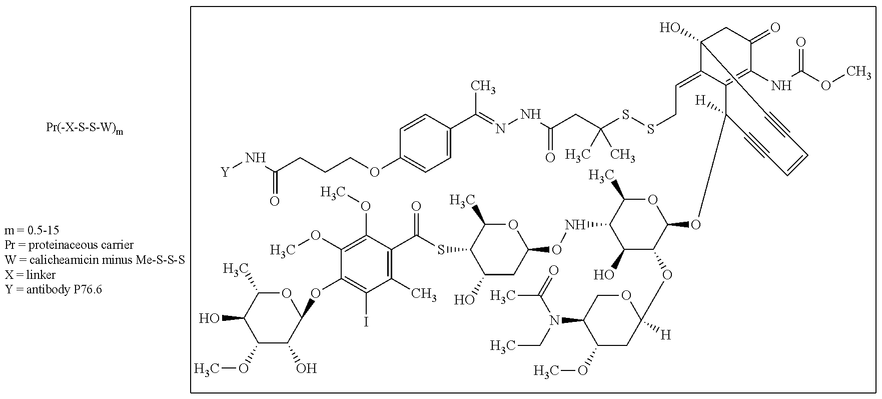

| | | | | |
|---|---|---|---|---|
| Calicheamicin- | not reported | non-Hodgkin | DNA | all human cancer; |
| IgG(CD22 antigen) | | lymphoma, cancer/ | cleaving | data not reported |
| conjugate/ | | not reported | agent | |
| semi-synthetic: | | | | |
| Micromonospora | | | | |
| echinospora/ | | | | |
| CMC-544 | | | | |

parially esterified polystyrene maleic acid copolymer (SMA)
conjugated to neocarzinostatin (NCS)

| | | | | |
|---|---|---|---|---|
| Neocarzinostatin¹⁷/ | 123760-07- | liver cancer and | DNA | cell culture data not |
| semi-synthetic; | 6; 9014- | brain cancer/ | cleaving | reported. |
| Streptomyces | 02-2 | not reported | agent | |
| carconistaticus/ | | | | |
| Zinostatin | | | | |
| stimalamer; YM-881; | | | | |
| YM-16881 | | | | |

IgG (TE-23)-conjugated to neocarzinostatin

| | | | | |
|---|---|---|---|---|
| Neocarzinostatin/ | not reported | solid tumors/ | DNA | cell culture data not |
| not reported/ | | toxicity not reported; | cleaving | reported. |
| TES-23-NCS | | the TES-23 antibody | agent and | |
| | | (without anticancer | immuno- | |
| | | agent) was as | stimulator | |
| | | effective at | | |
| | | eliminating tumors | | |
| | | as the drug | | |
| | | conjugated protein | | |

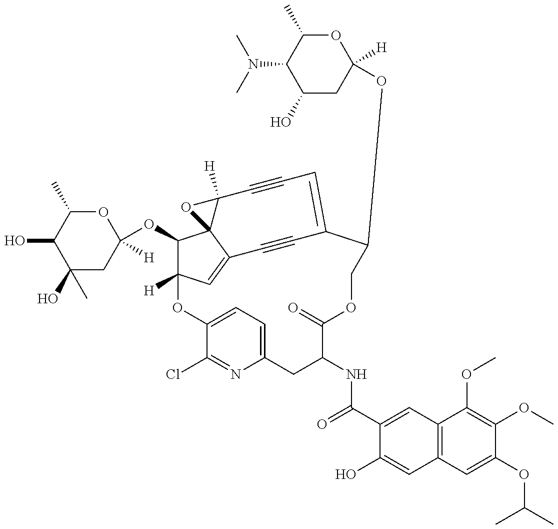

| | | | | |
|---|---|---|---|---|
| Kedarcidin¹⁸/ | 128512-40- | cancer/ | DNA | cell culture (IC50's |
| Streptoalloteichus sp | 3; 128512- | not reported | cleaving | in ng/mL), 0.4 |
| NOV strain L5856, | 39-0/ | | agent | HCT116; |
| ATCC 53650/ | chromophore | | | 0.3 HCT116/VP35; |
| NSC-646276 | and | | | 0.3 |
| | protein | | | HCT116/VM46; |
| | conjugate | | | 0.2 A2780; |
| | | | | 1.3 A2780/DDP. |
| | | | | animal models in |
| | | | | P388 and B-16 |
| | | | | melanoma. NCI |
| | | | | tumor panel, GI50's |
| | | | | from 50 μM to 5 |
| | | | | μM. |

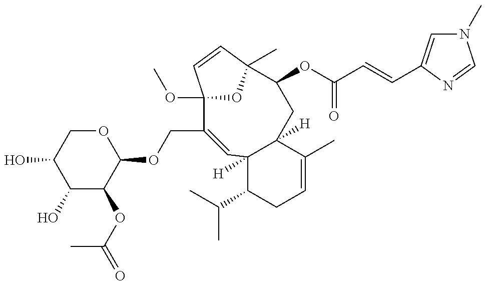 [SMILES: CO[C@]12C=C[C@](C)(O1)[C@@H](OC(=O)/C=C/c1cn(C)cn1)C[C@H]1C(C)=CC[C@H](C(C)C)[C@H]1/C=C\2CO[C@@H]1OC[C@@H](O)[C@@H](O)[C@@H]1OC(C)=O]

| | | | | |
|---|---|---|---|---|
| Eleutherobins/ | 174545-76- | cancer/ | tubulin | similar potency to |
| marine coral | 7/ | not reported | binding | taxol; not effective |
| | sarcodictyins | | agent | against MDR cell |
| | (marine | | | lines |
| | coral) | | | |

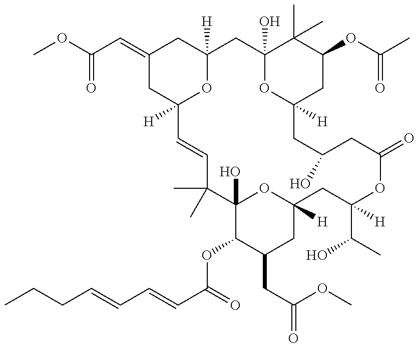 [SMILES: CCC/C=C/C=C/C(=O)O[C@H]1[C@H](CC(=O)OC)C[C@H]2C[C@H]([C@H](C)O)OC(=O)C[C@H](O)C[C@@H]3C[C@H](OC(C)=O)C(C)(C)[C@](O)(C[C@@H]4C/C(=C/C(=O)OC)C[C@H](/C=C/C(C)(C)[C@]1(O)O2)O4)O3]

| | | | | |
|---|---|---|---|---|
| Bryostatin-1/ | 83314-01-6 | leukemia, | immuno- | not reported |
| Bugula neritina | | melanoma, lung, | stimulant | |
| (marine bryosoan)/ | | cancer/ | (TNF, | |
| GMY-45618; NSC- | | myalgia; | GMCSF, | |
| 339555 | | accumulated | etc); | |
| | | toxicity; poor water | enhances | |
| | | solubility; dose | cell kill by | |
| | | limiting toxicity | current | |
| | | | anticancer | |
| | | | agents | |

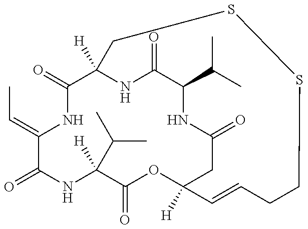 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

| | | | | |
|---|---|---|---|---|
| FR-901228/ | 128517-07- | leukemia, T-cell | histone | In vitro cell lines |
| Chromobacterium | 7 | lymphoma, cancer/ | deacetylase | (NCI tumor panel); |
| violaceum strain 968/ | | toxic doses (LD50) | inhibitor | IC50's of between |
| NSC-63-176; FK- | | 6.4 and 10 mg/Kg, | | 0.56 and 4.1 nM |
| 228 | | ip and iv | | (breast, lung, |
| | | respectively; GI | | gastric colon, |
| | | toxicity, lymphoid | | leukemia) |
| | | atrophy; dose | | |
| | | limiting toxicity | | |
| | | (human) 18 mg/Kg; | | |
| | | t1/2 of 8 hrs | | |
| | | (human) | | |

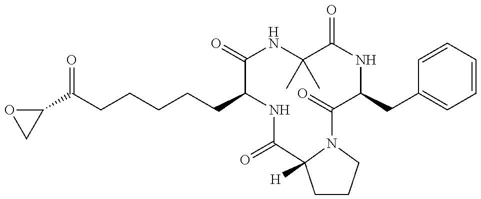 [SMILES: CC1(C)NC(=O)[C@H](CCCCCC(=O)[C@@H]2CO2)NC(=O)[C@H]2CCCN2C(=O)[C@H](Cc2ccccc2)NC1=O]

| | | | | |
|---|---|---|---|---|
| Chlamydocin/ | 53342-16-8 | cancer/ | histone | not reported (cell |
| not reported | | not reported | deacetylase | culture); |
| | | | inhibitor | inhibits histone |
| | | | | deacetylase at an |
| | | | | IC50 of 1.3 nM |

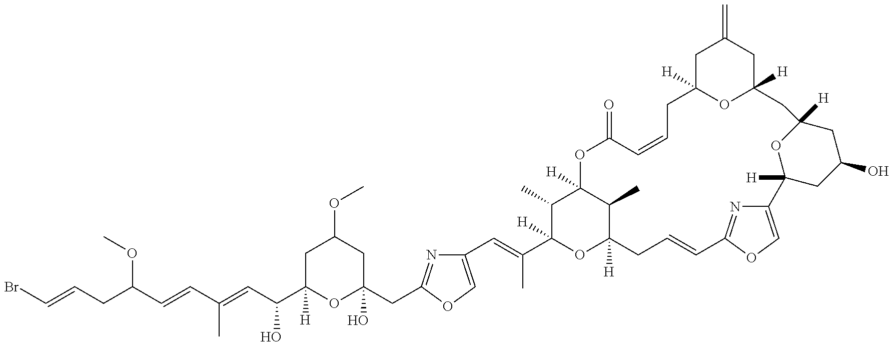 [SMILES: C=C1C[C@@H]2C[C@@H]3C[C@@H](O)C[C@@H](O3)c3coc(n3)/C=C/C[C@H]3O[C@@H](/C(C)=C/c4coc(C[C@]5(O)CC(OC)C[C@H]([C@H](O)/C=C(C)/C=C/C(C/C=C/Br)OC)O5)n4)[C@H](C)[C@@H](OC(=O)/C=C\C[C@@H](C1)O2)[C@H]3C]

| | | | | |
|---|---|---|---|---|
| Phorboxazole A¹⁹/ | 181377-57- | leukemia, myeloma/ | not | NCI tumor panel |
| marine sponge | 1; 165689- | not reported | reported | (details not |
| | 31-6; | | (induces | reported); |
| | 180911-82- | | apoptosis) | IC50's of 1-10 nM. |
| | 4; 165883- | | | The inhibition |
| | 76-1/ | | | values (clonogenic |
| | analogs | | | growth of human |
| | prepared | | | cancer cells) at 10 |
| | | | | nM ranged from 6.2 |
| | | | | to > 99.9% against |
| | | | | NALM-6 human B- |
| | | | | lineage acute |
| | | | | lymophoblastic |
| | | | | leukemia cells, BT- |
| | | | | 20 breast cancer |
| | | | | cells and U373 |
| | | | | glioblastoma cells, |
| | | | | with the specified |
| | | | | compound showing |
| | | | | inhibition values in |
| | | | | the range of 42.4 to |
| | | | | >99.9% against |
| | | | | these cell lines.; |
| | | | | IC50's are nM for |
| | | | | MDR cell lines. |

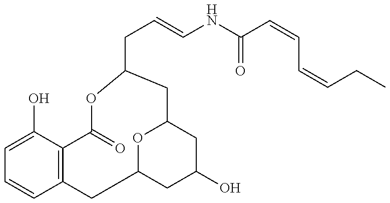 [SMILES: CC/C=C\C=C/C(=O)N/C=C/CC1CC2CC(O)CC(Cc3cccc(O)c3C(=O)O1)O2]

| | | | | |
|---|---|---|---|---|
| Apicularen A/ | 220757-06- | cancer/ | not | IC50's of 0.1 to 3 |
| Chondromyces | 2/ | not reported | reported | ng/mL (KB-3-A, |
| robustus | natural | | | KB-Va, K562, |
| | derivatives | | | HL60, U937, A498, |
| | | | | A549, PV3 and SK- |
| | | | | OV3) |

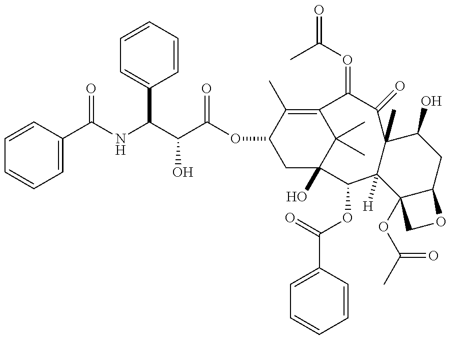

| | | | | |
|---|---|---|---|---|
| Taxol/ | 33069624/ | cancer; breast, | tubulin | NCI tumor panel; |
| Pacific yew and | many | prostate, ovary, | binding | GI50's of 3 nM to 1 |
| fungi/ | analogs | colon, lung, head & | agent | μM; |
| Paclitaxel; NSC- | | neck, etc./ | | TGI 50 nM to 25 |
| 125973 | | severe toxicity | | μM |
| | | (grade III and IV) | | |

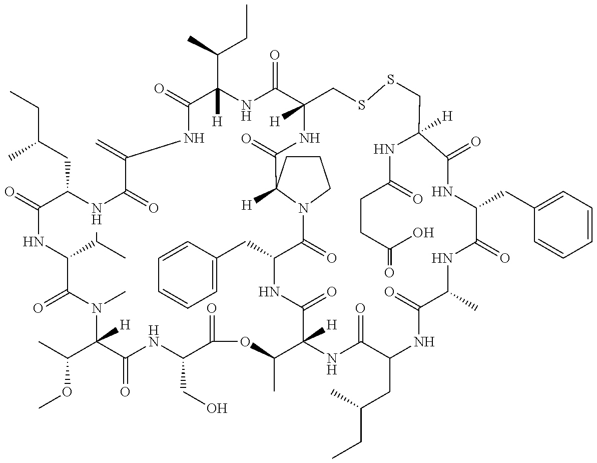 [SMILES: C=C1NC(=O)[C@H]([C@@H](C)CC)NC(=O)[C@@H]2CSSC[C@H](NC(=O)CCC(=O)O)C(=O)N[C@H](Cc3ccccc3)C(=O)N[C@H](C)C(=O)NC(C[C@@H](C)CC)C(=O)N[C@@H](C(=O)N[C@H](Cc3ccccc3)C(=O)N3CCC[C@H]3C(=O)N2)[C@@H](C)OC(=O)[C@H](CO)NC(=O)[C@H]([C@@H](C)OC)N(C)C(=O)[C@@H](C(C)C)NC(=O)[C@H](C[C@H](C)CC)NC1=O]

| | | | | |
|---|---|---|---|---|
| Vitilevuamide/ | 191681-63- | cancer/ | tubulin | cell culture; IC50's |
| Didemnum | 7 | not reported | binding | of 6-311 nM (panel |
| cuculliferum or | | | agent | of tumor cell lines |
| Polysyncraton | | | | HCT116 cells, |
| lithostrotum | | | | A549 cells, SK- |
| | | | | MEL-5 cells A498 |
| | | | | cells). The increase |
| | | | | in lifespan (ILS) for |
| | | | | CDFI mice after ip |
| | | | | injection of P388 |
| | | | | tumor cells was in |
| | | | | the range of −45 to |
| | | | | +70% over the dose |
| | | | | range of 0.13 to |
| | | | | 0.006 mg/kg. |

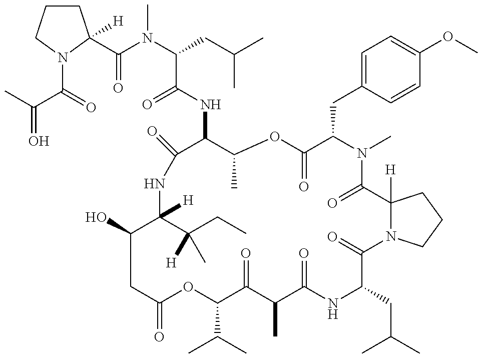

| | | | | |
|---|---|---|---|---|
| Didemnin B/ | 77327-05- | non-Hodgkin's | inhibits | NCI 60-tumor panel |
| Trididemnum | 0; 77327- | lymphoma, breast, | protein | (GI50's): 100 nM |
| solidum/ | 04-9; | carconoma, CNS, | synthesis | to 50 fM. |
| NSC-2325319; IND | 77327-06- | colon/ | via EF-1 | Not potent against |
| 24505 | 1/ | Discontinued due to | | MDR cell lines. |
| | other | cardiotoxicity; | | |
| | related | nausea, neuro- | | |
| | natural | muscular toxicity | | |
| | products | and vomiting MTD | | |
| | | 6.3 mg/Kg; toxicity | | |
| | | prevented achieving | | |
| | | a clinically signif. | | |
| | | effect; rapidly | | |
| | | cleared (t1/2 4.8 hrs | | |

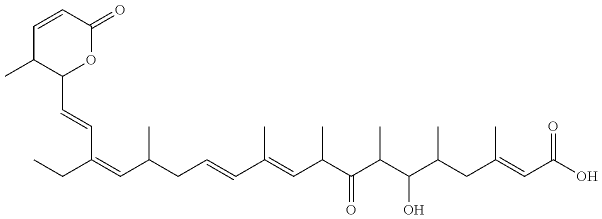 [SMILES: CCC(=C/C(C)C/C=C/C(C)=C/C(C)C(=O)C(C)C(O)C(C)C/C(C)=C/C(=O)O)/C=C/C1OC(=O)C=CC1C]

| | | |
|---|---|---|
| Leptomycin B/ | 87081-35-4 | NCI 60-tumor panel |
| Streptomyces sp. | | (GI50's): |
| strain ATS 1287/ | | 8 μM to 1 pM; |
| NSC-364372; | | (LC50): 250 μM to |
| elactocin | | 10 nM (several cell |
| | | lines at 0.1 nM). |
| | | Two testing results |
| | | with very different |
| | | potencies. |

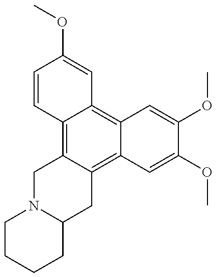 [SMILES: COc1ccc2c3c(c4cc(OC)c(OC)cc4c2c1)CC1CCCCN1C3]

| | |
|---|---|
| Cryptopleurin/ | NCI 60-tumor panel |
| not known/ | (GI50's): 19 nM to |
| NSC-19912 | 1 pM; (LC50): 40 |
| | μM to 10 nM |
| | (several cell lines at |
| | 1 pM). |

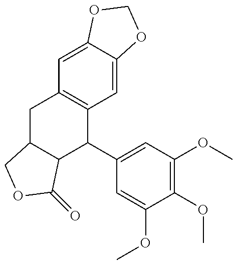 [SMILES: COc1cc(C2c3cc4c(cc3CC3COC(=O)C32)OCO4)cc(OC)c1OC]

| | | |
|---|---|---|
| Silicicolin/ | 19186-35-7 | NCI 60-tumor panel |
| not known/ | | (GI50's): ~100 nM |
| NSC-403148, | | to 3 nM; (LC50): |
| deoxypodophyllotoxin, | | 50 μM to 10 nM |
| desoxypodophyllotoxin | | |
| podophyllotoxin, | | |
| deoxysilicicolin | | |

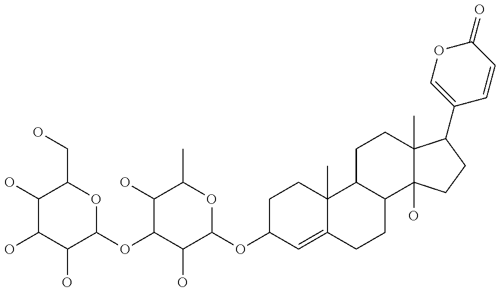 [SMILES: CC1OC(OC2C=C3CCC4C(CCC5(C)C(c6ccc(=O)oc6)CCC45O)C3(C)CC2)C(O)C(OC2OC(CO)C(O)C(O)C2O)C1O]

| | | |
|---|---|---|
| Scillaren A/ | 124-99-2 | NCI 60-tumor panel |
| not known/ | | (GI50's): 50 nM to |
| NSC-7525; Gluco- | | 0.1 nM; |
| proscillaridin A; | | (LC50): 250 μM to |
| Scillaren A | | 0.1 nM |

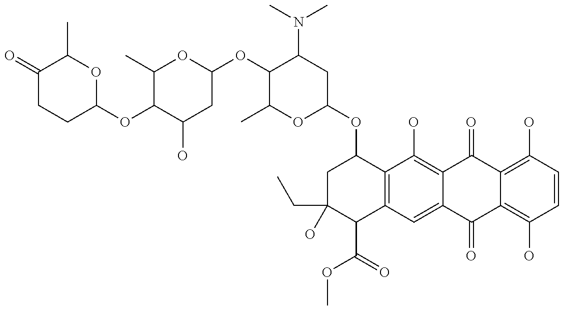 [SMILES: CCC1(O)CC(OC2CC(N(C)C)C(OC3CC(O)C(OC4CCC(=O)C(C)O4)C(C)O3)C(C)O2)c2c(cc3c(c2O)C(=O)c2c(O)ccc(O)c2C3=O)C1C(=O)OC]

| | | |
|---|---|---|
| Cinerubin A-HCl/ | not reported | NCI 60-tumor panel |
| not known/ | | (GI50's): 15 nM to |
| NSC-243022; | | 10 pM; (LC50): |
| Cinerubin A | | 100 μM to 6 nM |
| hydrochloride; | | |
| CL 86-F2-HCl; | | |
| CL-86-F2- | | |
| hydrochloride | | |

¹WO-09739025; U.S. Pat. No. 6,025,466;
²EP-00626383 30 Nov. 1994;
³JP-10101676;
⁴WO-09705162; WO-09717364 (dolastatin synthesis and analogs);
⁵Kosan licensed patent for Epothione analogs from Sloan-Kettering; US 00185968;
⁶WO-09723211;
⁷WO-09723211;
⁸JP-08092232;
⁹WO-09633211;
¹⁰EP-00608111; EP-00632042; EP-00634414; WO-09748278;
¹¹EP-00425235; JP53124692;
¹²US05416064; US06208020; EP-00425235B
¹³EP-004252341; JP-53124692; US-06333410B1
¹⁴JP-1104183
¹⁵EP-00689845
¹⁶EP-00689845
¹⁷EP-00136791; EP-00087957
¹⁸US 50001112; U.S. Pat. No. 5,143,906;
¹⁹WO-00136048

MAbs conjugated with a radioisotope are used as another means of treating human malignancies, particularly hematopoietic malignancies, with a high level of specificity and effectiveness. The most commonly used isotopes for therapy are the high-energy-emitters, such as ¹³¹I and ⁹⁰Y. Recently, ²¹³Bi-labeled anti-CD33 humanized MAb has also been tested in phase I human clinical trials. Reff et al., supra. In various embodiments, the therapeutic agent is a radioisotope.

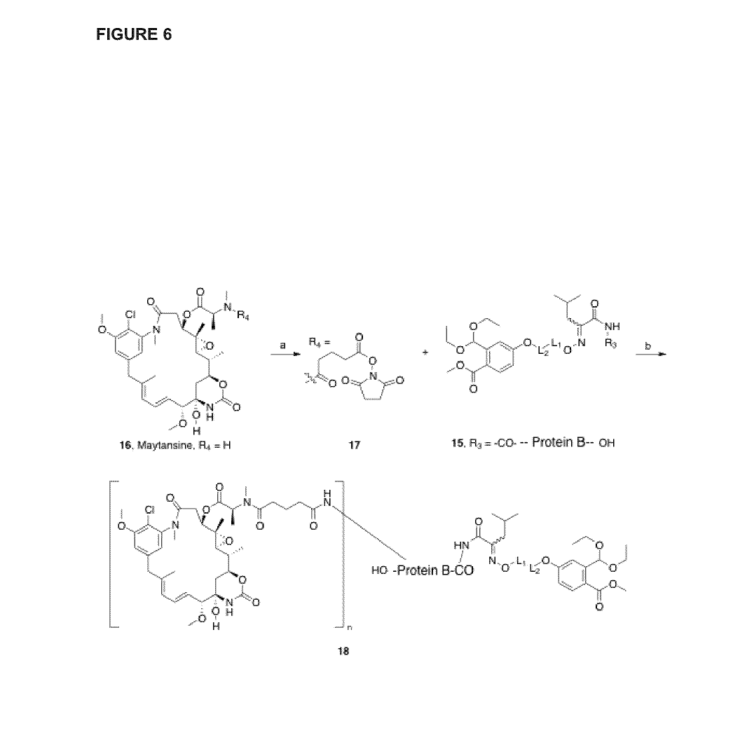

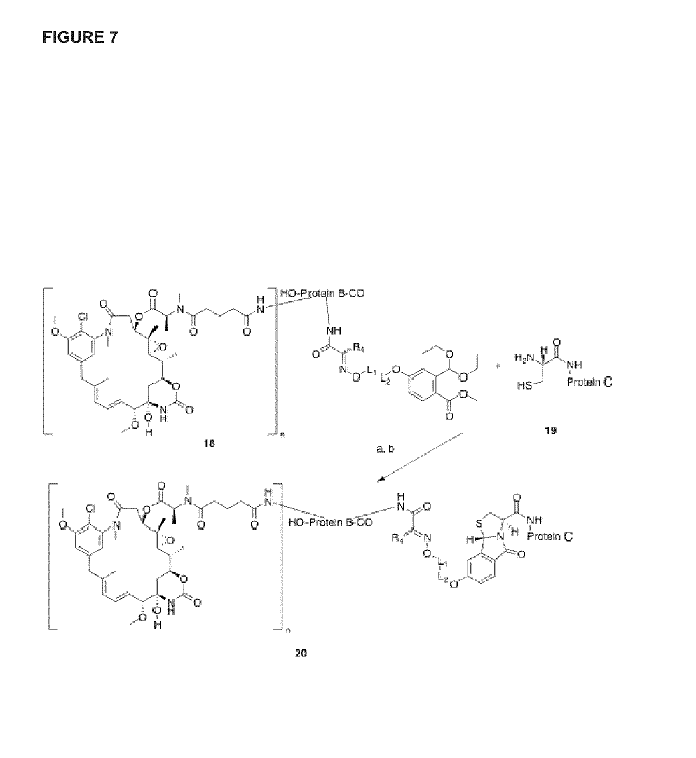

In an exemplary embodiment, the therapeutic agent is the toxin of FIG. 6. The toxin is conjugated to module (B) through any appropriate cleaveable or non-cleaveable linker of zero- or higher-order, such as that shown in FIG. 6 or FIG. 7. In an exemplary embodiment, module (B) is Domain III of albumin.

The conjugates of the invention can also include one or more linker at appropriate locations within the conjugate. An exemplary conjugate includes a linker between module (A) and module (B) to which it is conjugated. In various embodiments, the conjugate of the invention includes a linker between module (B) and module (C).

The art is replete with information regarding the structures of linkers for tethering two polypeptides or a polypeptide and a therapeutic moiety. Such information is readily accessible to and understandable by those of skill in the art. Such linkers are of use in assembling the conjugates of the instant invention. In an exemplary embodiment, the linker between (A) and (B), between (B) and (C) and a combination thereof is independently selected from a bond, a substituted or unsubstituted alkyl or a substituted or unsubstituted heteroalkyl moiety. Exemplary linkers further contain linkage fragments, such as those set forth hereinbelow, which are formed by the reaction of a first reactive functional group on a first conjugation partner (e.g., module (A)) and a second reactive functional group, of reactivity complementary to that of the first reactive functional group. In an exemplary embodiment, the first and second reactive functional groups are located at the terminal atom of the linker, or at precursors to the linker (i.e., the reactive functional groups become part of the linkage fragment on reaction). In an embodiment, the linker is hetero-bifunctional linker.

When the linker group, it can be selected from linker groups of from about 1 to about 20 atoms in length. In various embodiments, the linker has a substituted or unsubstituted hydrocarbon backbone. In various embodiments, the substituted or unsubstituted hydrocarbon backbone is interrupted by one or more heteroatom (e.g., O, N, S, P), i.e., a heteroalkyl linker.

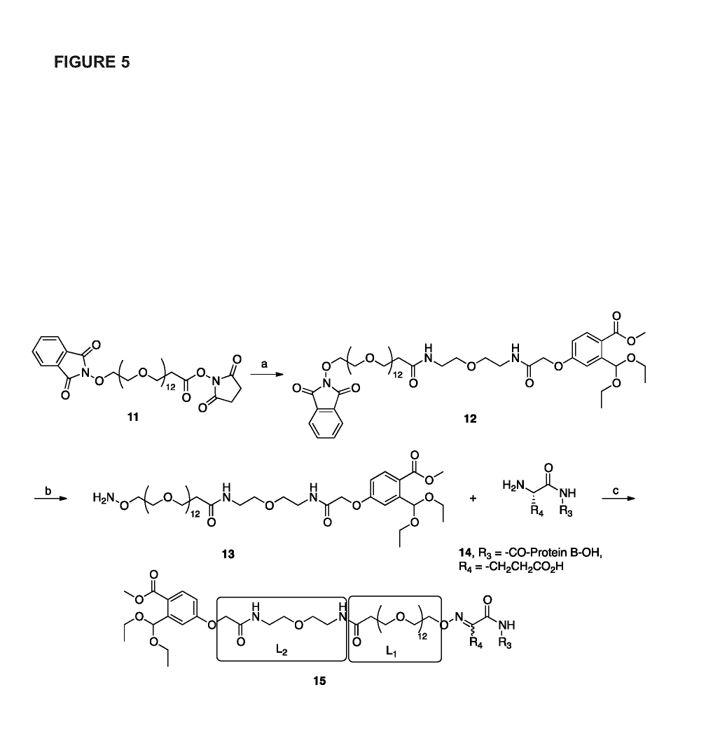

In various embodiments, the linker includes an alkylene oxide, e.g., poly(alkylene oxide), e.g., poly(ethylene glycol). In an exemplary embodiment according to FIG. 5—FIG. 7, one of L1 or L2 includes an alkylene oxide, e.g., ethylene oxide, and/or poly(ethylene glycol).

In an exemplary embodiment, the linker between module (A) and (B) is stable in the extracellular environment for a period of time sufficient to allow at least about 90%, about 80%, about 70%, about 60%, about 50% or at least about 40% of the conjugates administered to a subject, which are specifically localized on a target cell surface, to include essentially the same number of therapeutic agents (A) bound to module (B) as they had when they were administered to the subject. In other words, an exemplary linker is essentially uncleaved by the extracellular environment during the time the conjugate is resident in this environment. A further exemplary linker arm is cleavable in the intracellular environment but not to a degree that prevents a useful dosage of the intact conjugate being delivered to a target cell. Whether a linker is not substantially sensitive to the extracellular environment can be determined, for example, by incubating with plasma the antibody-drug conjugate compound for a predetermined time period (e.g., 2, 4, 8, 16, or 24 hours) and then quantitating the amount of free drug present in the plasma.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in 191P4D12-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker. Other examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the Val-Cit linker). One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In yet other embodiments, the linker unit is not cleavable and the drug is released by antibody degradation. (See U.S. Publication No. 2005/0238649 incorporated by reference herein in its entirety and for all purposes). Another example is succinimidyl-4-(N-maleimidomethyl)cyclohexane-1-carboxylate (SMCC).

In an exemplary embodiment, the linker between (A) and (B) is cleaved upon uptake of the conjugate (or a portion thereof) by the cell. A chemical solution to targeted delivery of cytotoxic or cytostatic drugs conjugated to cell-specific ligands is the “self-immolative linker”, PABC or PAB (para-aminobenzyloxycarbonyl), attaching the drug moiety to the ligand in the conjugate (Carl et al (1981) J. Med. Chem. 24:479-480; Chakravarty et al (1983) J. Med. Chem. 26:638-644). The PAB linker unit is also referred to as an electronic cascade spacer. The amide bond linking the carboxy terminus of a peptide unit and the para-aminobenzyl of PAB may be a substrate and cleavable by certain proteases. The aromatic amine becomes electron-donating and initiates an electronic cascade that leads to the expulsion of the leaving group, which releases the free drug after elimination of carbon dioxide (de Groot, et al (2001) Journal of Organic Chemistry 66(26):8815-8830). Cathepsin B is a ubiquitous cysteine protease. It is an intracellular enzyme, except in pathological conditions, such as metastatic tumors (Sinha et al (2001) Prostate 49:172-184) or rheumatoid arthritis (Hashimoto et al (2001) Biochem. Biophys. Res. Commun. 283:334-339). Therefore, conjugates produced with cathepsin B-cleavable linkers are likely to be stable in circulation. Upon cleavage of a peptide bond adjacent to the PABC, i.e. by an intracellular enzyme, the drug is released from the ligand whereby no remaining portion of the linker is bound (de Groot, et al (2002) Molecular Cancer Therapeutics 1(11):901-911; de Groot, et al (1999) J. Med. Chem. 42(25):5277-5283).

Linkers containing the para-aminobenzyloxycarbonyl (PAB or PABC) unit, in conjunction with a peptide unit, have been developed with a “self-immolating” or “self-immolative” mechanism of 1,6 elimination and fragmentation under enzymatic, hydrolytic, or other metabolic conditions to release a drug moiety from a targeting ligand, such as an antibody (U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). The 2-nitroimidazol-5-ylmethyl group has been reported as a fragmenting prodrug unit (Hay et al. (1999) Bioorg. Med. Chem. Lett. 9:2237). For the use of the PAB unit in prodrugs and conjugates, see also: Walker, et al (2004) Bioorganic & Medicinal Chemistry Letters 14(16):4323-4327; Devy, et al (2004) FASEB Journal 18(3):565-567, 10.1096/fⱼ0.03-0462fje; Francisco, et al Blood (2003) 102(4):1458-1465; Doronina, et al (2003) Nature Biotechnology 21(7):778-784; King, et al (2002) Journal of Medicinal Chemistry 45(19):4336-4343; Dubowchik, et al (2002) Bioconjugate Chemistry 13(4):855-869; Dubowchik, et al (2002) Bioorganic & Medicinal Chemistry Letters 12(11):1529-1532.

### Reactive Functional Groups

The conjugates of the invention are assembled from covalent bonding reactions between precursors bearing a reactive functional group, which is a locus for formation of a covalent bond between the precursors. The precursors of conjugates of the invention bear a reactive functional group, which can be located at any position on the compound (e.g., therapeutic agent and/or polypeptide). The finished conjugates can include a further reactive functional group at any point on the molecule. In various embodiments, a reactive functional group on the therapeutic agent (or a linker attached to the therapeutic agent) is reacted with a reactive functional group on the polypeptide molecule (or a linker attached to the polypeptide molecule) to couple the two components together covalently through a linkage fragment. When the polypeptide is polyvalent, presenting multiple reactive functional groups for conjugation with a therapeutic species, the multiple reactive functional groups can be the same or different.

Exemplary species include a reactive functional group attached to an alkyl or heteroalkyl moiety on the therapeutic moiety (or polypeptide). When the reactive group is attached a substituted or unsubstituted alkyl or substituted or unsubstituted heteroalkyl linker moiety, the reactive group is preferably located at a terminal position of the alkyl or heteroalkyl chain. Reactive groups and classes of reactions useful in practicing the present invention are generally those that are well known in the art of bioconjugate chemistry. Currently favored classes of reactions available with reactive compounds of the invention are those proceeding under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D. C., 1982.

Useful reactive functional groups include, for example:
(a) carboxyl groups and derivatives thereof including, but not limited to activated esters, e.g., N-hydroxysuccinimide esters, N-hydroxyphthalimide, N-hydroxybenztriazole esters, p-nitrophenyl esters; acid halides; acyl imidazoles; thioesters; alkyl, alkenyl, alkynyl and aromatic esters; and activating groups used in peptide synthesis;
(b) hydroxyl groups and hydroxylamines, which can be converted to esters, sulfonates, phosphoramidates, ethers, aldehydes, etc.
(c) haloalkyl groups, wherein the halide can be displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups, which are capable of participating in Diels-Alder reactions such as, for example, maleimido groups;
(e) aldehyde or ketone groups, allowing derivatization via formation of carbonyl derivatives, e.g., imines, hydrazones, semicarbazones or oximes, or via such mechanisms as Grignard addition or alkyllithium addition;
(f) sulfonyl halide groups for reaction with amines, for example, to form sulfonamides;
(g) thiol groups, which can be converted to disulfides or reacted with acyl halides, for example;
(h) amine, hydrazine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds; and
(k) phosphoramidites and other standard functional groups useful in nucleic acid synthesis.

In various embodiments, the reactive functional group is a member selected from:

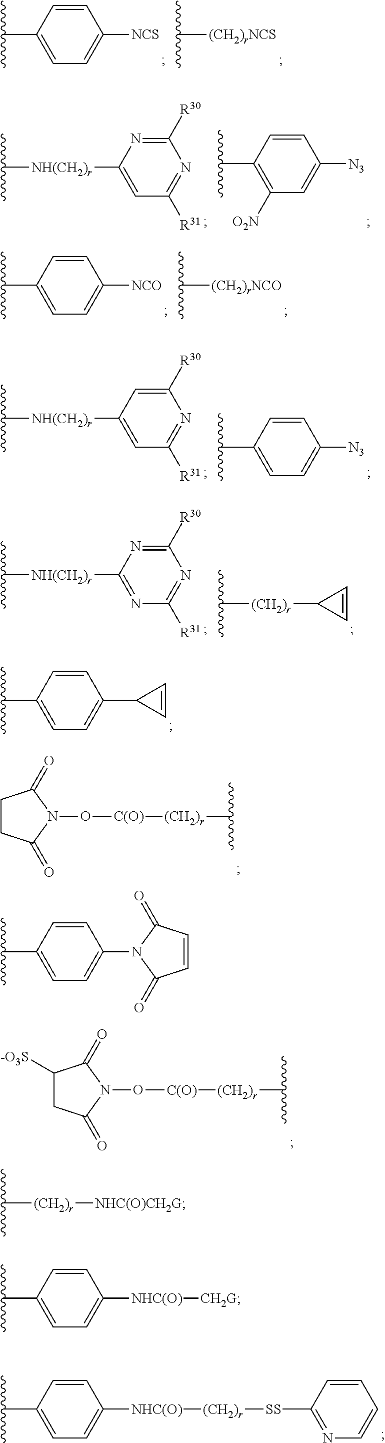

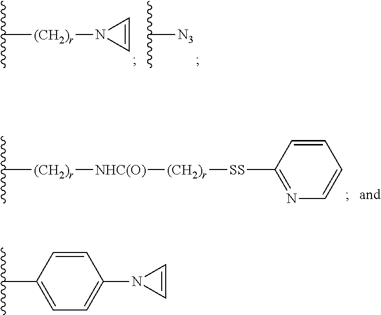

in which each r is independently selected from the integers from 1 to 10; G is a halogen; and R³⁰ and R³¹ are members independently selected from H and halogen and at least one of R³⁰ and R³¹ is halogen.

The reactive functional groups can be chosen such that they do not participate in, or interfere with, the reactions necessary to assemble or utilize the reactive therapeutic agent precursor or the reactive polypeptide conjugation partner for the therapeutic agent precursor. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

Exemplary linkers of use in the present invention include substituted or unsubstituted alkyl, and substituted or unsubstituted heteroalkyl linkers. An exemplary linker of use in the invention includes a poly(ether), e.g., poly(alkylene oxide), e.g., poly(ethylene glycol).

In various embodiments, the therapeutic moiety and the polypeptide are conjugated through through a linkage fragment either directly or through a linker. Exemplary linkage fragments include a bond and a moiety that includes at least one heteroatom, which is formed by the reaction of two reactive functional groups of complementary reactivity. Exemplary linkage fragments of use in the conjugates of the invention include, without limitation:
—(CH₂)ₜS(CH₂)_{z}—, —(CH₂)ₓSC(O)NR(CH₂)_{z}—, —(CH₂)ₓSC(O)O(CH₂)_{z}—, —(CH₂)ₓNR(CH₂)_{z}—, —(CH₂)—NRC(O)(CH₂)_{z}—, —(CH₂)—NRC(O)O(CH₂)_{z}—, —(CH₂)ₓO(CH₂)_{z}—, —(CH₂)ₒT-PEG-, —(CH₂)—C(O)CH₂S—, —S-maleimide-N—, —RNC(O)NR—, —RNS(O)NR—, —S(O)₂NR—,
   wherein T is a member selected from S, NH, NHC(O), C(O)NH, NHC(O)O, OC(O)NH, and O. The index o is an integer from 1 to 50; and the indices t and z are independently selected from the integers from 0 to 10. The linkage fragments can also be formed via “Click Chemistry between one component having an azide moiety and another component with an alkyne moitey. The therapeutic agent can be derivatized with either reactive functional group as can the polypeptide.

Additional Linkage Fragments Include the Structures:

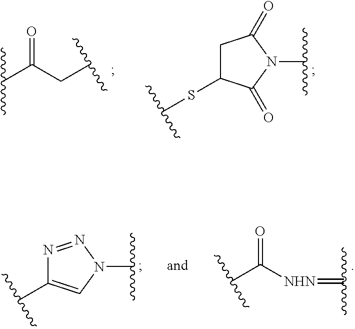

FIG. 5-FIG. 7 provide examples of additional linkage fragments in the context of L1 and L2 of these figures.

In another exemplary embodiment, the linker moiety includes at least one bond that is degraded in vivo, releasing the therapeutic polypeptide from the targeting agent, following delivery of the conjugate to the targeted tissue or region of the body. Many cleavable groups are known in the art. See, for example, Jung et al., Biochem. Biophys. Acta 761:152-162 (1983); Joshi et al., J. Biol. Chem. 265:14518-14525 (1990); Zarling et al., J. Immunol. 124:913-920 (1980); Bouizar et al., Eur. J. Biochem. 155:141-147 (1986); Park et al., J. Biol. Chem. 261:205-210 (1986); Browning et al., J. Immunol. 143:1859-1867 (1989). Moreover a broad range of cleavable, bifunctional (both homo- and hetero-bifunctional) linker groups is commercially available from suppliers such as Pierce.

Exemplary cleavable moieties can be cleaved using light, heat or reagents such as thiols, hydroxylamine, bases, periodate and the like. Moreover, certain preferred groups are cleaved in vivo in response to being endocytosed (e.g., cis-aconityl; see, Shen et al., Biochem. Biophys. Res. Commun. 102:1048 (1991)). Preferred cleavable groups comprise a cleavable moiety which is a member selected from the group consisting of disulfide, ester, imide, carbonate, nitrobenzyl, phenacyl and benzoin groups.

### Determination of Therapeutic Agent Loading

Therapeutic agent (“drug”) loading refers to the average number of therapeutic agent moieties per antibody in a molecule. Drug loading (p) may range from 1 to 20 drug moieties (A) per antibody. PDCs of the invention include antibodies conjugated with a range of drug moieties, from p of about 1 to about 20. Depending on the linking chemistry employed in the design of PDC, the reaction product profile could involve a heterogenous combination of PDC molecules with this range of drug moieties. A small fraction of the starting protein material could also remain unconjugated in the product. The average number of drug moieties per antibody in preparations of PDC from conjugation reactions may be characterized by conventional means such as mass spectroscopy, various types of capillary isoelectric focusing techniques such as iCIEF, spectroscopic techniques such as UV spectroscopy, chromatographic techniques such as hydrophobic interaction or ion exchange chromatography, and ELISA assay. The quantitative distribution of PDC in terms of p may also be determined. In some instances, separation, purification, and characterization of homogeneous PDC where p is a certain value from PDC with other drug loadings may be achieved by means such as electrophoresis.

For some antibody-drug conjugates, loading may be limited by the number of attachment sites on the antibody. For example, where the attachment is a cysteine thiol, an antibody may have only one or several cysteine thiol groups, or may have only one or several sufficiently reactive thiol groups through which a linker may be attached. In certain embodiments, higher drug loading, e.g. p>5, may cause aggregation, insolubility, toxicity, or loss of cellular permeability of certain antibody-drug conjugates. In certain embodiments, the drug loading for an PDC of the invention ranges from 1 to about 8; from about 2 to about 6; from about 3 to about 5; from about 3 to about 4.

In certain embodiments, fewer than the theoretical maximum of drug moieties are conjugated to an antibody during a conjugation reaction. An antibody may contain, for example, lysine residues that do not react with the drug-linker intermediate or linker reagent. Generally, antibodies do not contain many free and reactive cysteine thiol groups which may be linked to a drug moiety; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine.

The loading (drug/antibody ratio) of n PDC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488.

It is to be understood that where more than one nucleophilic group reacts with a drug-linker intermediate or linker reagent followed by drug moiety reagent, then the resulting product is a mixture of PDC compounds with a distribution of one or more drug moieties attached to an antibody. The average number of drugs per antibody may be calculated from the mixture by a dual ELISA antibody assay, which is specific for antibody and specific for the drug. Individual PDC molecules may be identified in the mixture by mass spectroscopy and separated by HPLC, e.g. hydrophobic interaction chromatography (see, e.g., Hamblen, K J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous PDC with a single loading value may be isolated from the conjugation mixture by electrophoresis or chromatography.

### Methods of Determining Cytotoxic Effect of PDCs

Methods of determining whether a therapeutic agent or PDC exerts a cytostatic and/or cytotoxic effect on a cell are known. Generally, the cytotoxic or cytostatic activity of an PDC can be measured by: exposing mammalian cells expressing a target protein of the PDC in a cell culture medium; culturing the cells for a period from about 6 hours to about 5 days; and measuring cell viability. Cell-based in vitro assays can be used to measure viability (proliferation), cytotoxicity, and induction of apoptosis (caspase activation) of the PDC.

For determining whether an PDC exerts a cytostatic effect, a thymidine incorporation assay may be used. For example, cancer cells expressing a target antigen at a density of 5,000 cells/well of a 96-well plated can be cultured for a 72-hour period and exposed to 0.5 .mu.Ci of sup.3H-thymidine during the final 8 hours of the 72-hour period. The incorporation of .sup.3H-thymidine into cells of the culture is measured in the presence and absence of the PDC.

For determining cytotoxicity, necrosis or apoptosis (programmed cell death) can be measured. Necrosis is typically accompanied by increased permeability of the plasma membrane; swelling of the cell, and rupture of the plasma membrane. Apoptosis is typically characterized by membrane blebbing, condensation of cytoplasm, and the activation of endogenous endonucleases. Determination of any of these effects on cancer cells indicates that an PDC is useful in the treatment of cancers.

Cell viability can be measured by determining in a cell the uptake of a dye such as neutral red, trypan blue, or ALAMAR®. blue (see, e.g., Page et al., 1993, Intl. J. Oncology 3:473-476). In such an assay, the cells are incubated in media containing the dye, the cells are washed, and the remaining dye, reflecting cellular uptake of the dye, is measured spectrophotometrically. The protein-binding dye sulforhodamine B (SRB) can also be used to measure cytoxicity (Skehan et al., 1990, J Natl. Cancer Inst. 82:1107-12).

Alternatively, a tetrazolium salt, such as MTT, is used in a quantitative colorimetric assay for mammalian cell survival and proliferation by detecting living, but not dead, cells (see, e.g., Mosmann, 1983, J Immunol. Methods 65:55-63).

Apoptosis can be quantitated by measuring, for example, DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, 1999, no. 2, pp. 34-37 (Roche Molecular Biochemicals).

Apoptosis can also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring apoptotic cell number has been described by Duke and Cohen, Current Protocols in Immunology (Coligan et al. eds., 1992, pp. 3.17.1-3.17.16). Cells also can be labeled with a DNA dye (e.g., acridine orange, ethidium bromide, or propidium iodide) and the cells observed for chromatin condensation and margination along the inner nuclear membrane. Other morphological changes that can be measured to determine apoptosis include, e.g., cytoplasmic condensation, increased membrane blebbing, and cellular shrinkage.

The presence of apoptotic cells can be measured in both the attached and “floating” compartments of the cultures. For example, both compartments can be collected by removing the supernatant, trypsinizing the attached cells, combining the preparations following a centrifugation wash step (e.g., 10 minutes at 2000 rpm), and detecting apoptosis (e.g., by measuring DNA fragmentation). (See, e.g., Piazza et al., 1995, Cancer Research 55:3110-16).

In vivo, the effect of a 191P4D12 therapeutic composition can be evaluated in a suitable animal model. For example, xenogenic cancer models can be used, wherein cancer explants or passaged xenograft tissues are introduced into immune compromised animals, such as nude or SCID mice (Klein et al., 1997, Nature Medicine 3: 402-408). For example, PCT Patent Application WO98/16628 and U.S. Pat. No. 6,107,540 describe various xenograft models of human prostate cancer capable of recapitulating the development of primary tumors, micrometastasis, and the formation of osteoblastic metastases characteristic of late stage disease. Efficacy can be predicted using assays that measure inhibition of tumor formation, tumor regression or metastasis, and the like.

In vivo assays that evaluate the promotion of apoptosis are useful in evaluating therapeutic compositions. In one embodiment, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

### The Polypeptides

The invention is exemplified by reference to the use of exemplary therapeutic antibodies as targeting module (B). Exemplary therapeutic antibodies (and their abbreviations) include: Herceptin® (trastuzumab)=full length, humanized antiHER2 (MW 145167), Herceptin F(ab′)2=derived from antiHER2 enzymatically (MW 100000), 4D5=full-length, murine antiHER2, from hybridoma, rhu4D5=transiently expressed, full-length humanized antibody, rhuFab4D5=recombinant humanized Fab (MW 47738), 4D5Fc8=full-length, murine antiHER2, with mutated FcRn binding domain.

The antibody of the protein-drug conjugates (PDC) of the invention may specifically bind to a receptor encoded by an ErbB gene. The antibody may bind specifically to an ErbB receptor selected from EGFR, HER2, HER3 and HER4. The PDC may specifically bind to the extracellular domain of the HER2 receptor and inhibit the growth of tumor cells which overexpress HER2 receptor. HERCEPTIN® (trastuzumab) selectively binds to the extracellular domain (ECD) of the human epidermal growth factor receptor2 protein, HER2 (ErbB2) (U.S. Pat. Nos. 5,821,337; 6,054,297; 6,407,213; 6,639,055; Coussens et al (1985) Science 230:1132-9; Slamon, et al (1989) Science 244:707-12). Trastuzumab is an IgG1 kappa antibody that contains human framework regions with the complementarily-determining regions (cdr) of a murine antibody (4D5) that binds to HER2. Trastuzumab binds to the HER2 antigen and thus inhibits the proliferation of human tumor cells that overexpress HER2 (Hudziak R M, et al (1989) Mol Cell Biol 9:1165-72; Lewis G D, et al (1993) Cancer Immunol Immunother; 37:255-63; Baselga J, et al (1998) Cancer Res. 58:2825-2831).

The antibody of the PDC may be a monoclonal antibody, e.g. a murine monoclonal antibody, a chimeric antibody, or a humanized antibody. A humanized antibody may be huMAb4D5-1, huMAb4D5-2, huMAb4D5-3, huMAb4D5-4, huMAb4D5-5, huMAb4D5-6, huMAb4D5-7 or huMAb4D5-8 (Trastuzumab). The antibody may be an antibody fragment, e.g. a Fab fragment.

Known antibodies for the treatment or prevention of cancer can be conjugated according to the platform and methods of the invention. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, humanized anti-HER2 monoclonal antibody for the treatment of patients with metastatic breast cancer; RITUXAN® (rituximab; Genentech) which is a chimeric anti-CD20 monoclonal antibody for the treatment of patients with non-Hodgkin's lymphoma; OvaRex (AltaRex Corporation, MA) which is a murine antibody for the treatment of ovarian cancer; Panorex (Glaxo Wellcome, N.C.) which is a murine IgG₂ₐ antibody for the treatment of colorectal cancer; Cetuximab Erbitux (Imclone Systems Inc., NY) which is an anti-EGFR IgG chimeric antibody for the treatment of epidermal growth factor positive cancers, such as head and neck cancer; Vitaxin (MedImmune, Inc., MD) which is a humanized antibody for the treatment of sarcoma; Campath I/H (Leukosite, MA) which is a humanized IgG₁ antibody for the treatment of chronic lymphocytic leukemia (CLL); Smart MI95 (Protein Design Labs, Inc., CA) which is a humanized anti-CD33 IgG antibody for the treatment of acute myeloid leukemia (AML); LymphoCide (Immunomedics, Inc., NJ) which is a humanized anti-CD22 IgG antibody for the treatment of non-Hodgkin's lymphoma; Smart ID10 (Protein Design Labs, Inc., CA) which is a humanized anti-HLA-DR antibody for the treatment of non-Hodgkin's lymphoma; Oncolym (Techniclone, Inc., CA) which is a radiolabeled murine anti-HLA-Dr10 antibody for the treatment of non-Hodgkin's lymphoma; Allomune (BioTransplant, CA) which is a humanized anti-CD2 MAb for the treatment of Hodgkin's Disease or non-Hodgkin's lymphoma; Avastin (Genentech, Inc., CA) which is an anti-VEGF humanized antibody for the treatment of lung and colorectal cancers; Epratuzamab (Immunomedics, Inc., NJ and Amgen, CA) which is an anti-CD22 antibody for the treatment of non-Hodgkin's lymphoma; and CEAcide (Immunomedics, NJ) which is a humanized anti-CEA antibody for the treatment of colorectal cancer.

Hybrid or bifunctional antibodies may be derived, as noted, either biologically, by cell fusion techniques, or chemically, especially with cross-linking agents or disulfide bridge-forming reagents, and may be comprised of whole antibodies and/or fragments thereof (EP 0105360). Methods for obtaining such hybrid antibodies are disclosed, for example, in WO 83/03679, and EP 0217577, both of which are incorporated herein by reference. Bifunctional antibodies include those biologically prepared from a “polydoma” or “quadroma” or which are synthetically prepared with cross-linking agents such as bis-(maleimido)-methyl ether (“BMME”), or with other cross-linking agents familiar to those skilled in the art.

Immunoglobulin antibodies can recognize a tumor-associated antigen. As used, “immunoglobulin” may refer to any recognized class or subclass of immunoglobulins such as IgG, IgA, IgM, IgD, or IgE. The immunoglobulin can be derived from any species, such as human, murine, or rabbit origin. Further, the immunoglobulin may be polyclonal, monoclonal, or fragments. Such immunoglobulin fragments may include, for example, the Fab′, F(ab′)₂, FAT or Fab fragments, or other antigen recognizing immunoglobulin fragments. Such immunoglobulin fragments can be prepared, for example, by proteolytic enzyme digestion, for example, by pepsin or papain digestion, reductive alkylation, or recombinant techniques. The materials and methods for preparing such immunoglobulin fragments are well-known to those skilled in the art (Parham, (1983) J. Immunology, 131:2895; Lamoyi et al., (1983) J. Immunological Methods, 56:235; Parham, (1982) J. Immunological Methods, 53:133; and Matthew et al., (1982) J. Immunological Methods, 50:239).

In addition the immunoglobulin may be a single chain antibody (“SCA”). These may consist of single chain Fv fragments (“scFv”) in which the variable light (“V L”) and variable heavy (“V H”) domains are linked by a peptide bridge or by disulfide bonds. Also, the immunoglobulin may consist of single V H domains (dAbs) which possess antigen-binding activity. See, e.g., G. Winter and C. Milstein, Nature, 349, 295 (1991); R. Glockshuber et al., Biochemistry 29, 1362 (1990); and, E. S. Ward et al., Nature 341, 544 (1989).

The immunoglobulin can be a chimeric antibody, e.g. humanized antibodies. Also, the immunoglobulin may be a “bifunctional” or “hybrid” antibody, that is, an antibody which may have one arm having a specificity for one antigenic site, such as a tumor associated antigen while the other arm recognizes a different target, for example, a hapten which is, or to which is bound, an agent lethal to the antigen-bearing tumor cell. Alternatively, the bifunctional antibody may be one in which each arm has specificity for a different epitope of a tumor associated antigen of the cell to be therapeutically or biologically modified. In any case, the hybrid antibodies may have dual specificity, with one or more binding sites specific for the hapten of choice or one or more binding sites specific for a target antigen, for example, an antigen associated with a tumor, an infectious organism, or other disease state.

One skilled in the art will recognize that a bifunctional-chimeric antibody can be prepared which would have the benefits of lower immunogenicity of the chimeric or humanized antibody, as well as the flexibility, especially for therapeutic treatment, of the bifunctional antibodies described above. Such bifunctional-chimeric antibodies can be synthesized, for instance, by chemical synthesis using cross-linking agents and/or recombinant methods of the type described above. In any event, the present invention should not be construed as limited in scope by any particular method of production of an antibody whether bifunctional, chimeric, bifunctional-chimeric, humanized, or an antigen-recognizing fragment or derivative thereof.

In addition, the invention encompasses within its scope immunoglobulins (as defined above) or immunoglobulin fragments to which are fused active proteins, for example, an enzyme of the type disclosed in Neuberger, et al., PCT application, WO86/01533, published Mar. 13, 1986. The disclosure of such products is incorporated herein by reference.

As noted, “bifunctional”, “fused”, “chimeric” (including humanized), and “bifunctional-chimeric” (including humanized) antibody constructions also include, within their individual contexts constructions comprising antigen recognizing fragments. Such fragments could be prepared by traditional enzymatic cleavage of intact bifunctional, chimeric, humanized, or chimeric-bifunctional antibodies. If, however, intact antibodies are not susceptible to such cleavage, because of the nature of the construction involved, the noted constructions can be prepared with immunoglobulin fragments used as the starting materials; or, if recombinant techniques are used, the DNA sequences, themselves, can be tailored to encode the desired “fragment” which, when expressed, can be combined in vivo or in vitro, by chemical or biological means, to prepare the final desired intact immunoglobulin “fragment”. It is in this context, therefore, that the term “fragment” is used.

Furthermore, as noted above, the immunoglobulin (antibody), or fragment thereof, used in the present invention may be polyclonal or monoclonal in nature. The preparation of such polyclonal or monoclonal antibodies now is well known to those skilled in the art who, of course, are fully capable of producing useful immunoglobulins which can be used in the invention. See, e.g., G. Kohler and C. Milstein, Nature 256, 495 (1975). In addition, hybridomas and/or monoclonal antibodies which are produced by such hybridomas and which are useful in the practice of the present invention are publicly available from sources such as the American Type Culture Collection (“ATCC”) 12301 Parklawn Drive, Rockville, Md. 20852 or, commercially, for example, from Boehringer-Mannheim Biochemicals, P.O. Box 50816, Indianapolis, Ind. 46250.

The present invention can be practiced with essentially any antibody or fragment thereof. In an exemplary embodiment, the polypeptide is a humanized antibody or fragment thereof. Exemplary humanized monoclonal antibodies with therapeutic potential as chemotherapeutic agents in the conjugates of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

The present invention should also be construed to encompass conjugates of “derivatives,” “mutants”, and “variants” of the polypeptides of the invention (or of the DNA encoding the same) which derivatives, mutants, and variants are polypeptides which are altered in one or more amino acids (or, when referring to the nucleotide sequence encoding the same, are altered in one or more base pairs) such that the resulting polypeptide (or DNA) is not identical to the sequences recited herein, but has the same biological property as the polypeptides disclosed herein, in that the polypeptide has biological/biochemical properties.

Further included are conjugates formed with fragments of antibodies that retain the desired biological activity of the antibody irrespective of the length of the polypeptide. It is well within the skill of the artisan to isolate smaller than full length forms of any of the polypeptides useful in the invention, and to determine, using the assays known in the art, which isolated fragments retain a desired biological activity and are therefore useful polypeptides in the invention.

A biological property of a polypeptide of use in the conjugates of the present invention should be construed to include, but not be limited to including, the ability of the polypeptide to function in the biological assay and environments described herein, such as reduction of inflammation, elicitation of an immune response, blood-clotting, increased hematopoietic output, protease inhibition, immune system modulation, binding an antigen, growth, alleviation of treatment of a disease, DNA cleavage, and the like.

Exemplary antigen binding constructs described herein includes a Fc. The Fc includes two Fc polypeptides each having a CH3 domain for dimerization. The N-terminal end of each Fc polypeptide is linked to the C-terminus of one of the antigen binding polypeptide constructs with or without a linker. Such constructs are described in further detail in PCT/US2014/037401, the disclosure of which is incorporated herein by reference.

In one embodiment, the Fc is an IgG1 Fc construct, an IgG2 Fc construct, an IgG3 Fc construct, or an IgG4 Fc construct.

In some embodiments, at least one CH3 domain has at least one amino acid modification that promotes the formation of a heterodimeric Fc with stability comparable to a wild-type homodimeric Fc. Exemplary modifications are described below. In some embodiments, the dimerized CH3 domains of the heterodimeric Fc have a melting temperature (Tm) as measured by differential scanning calorimetry (DSC) of about 68, 69, 70, 71, 72, 73, 74, 75, 76, 77, 77.5, 78, 79, 80, 81, 82, 83, 84, or 85° C. or higher. In some embodiments, the dimeric Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when produced; or wherein the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when expressed or when expressed via a single cell.

In some aspects, the Fc comprises one or more modifications in at least one of the C_{H3} sequences. In some aspects, the Fc comprises one or more modifications in at least one of the C_{H2} sequences.

In some aspects, Fc is a Fc described in patent applications PCT/CA2011/001238, filed Nov. 4, 2011 or PCT/CA2012/050780, filed Nov. 2, 2012, the entire disclosure of each of which is hereby incorporated by reference in its entirety for all purposes.

In some aspects, a construct described herein comprises a heterodimeric Fc comprising a modified CH3 domain that has been asymmetrically modified. The heterodimeric Fc can comprise two heavy chain constant domain polypeptides: a first heavy chain polypeptide and a second heavy chain polypeptide, which can be used interchangeably provided that Fc comprises one first heavy chain polypeptide and one second heavy chain polypeptide. Generally, the first heavy chain polypeptide comprises a first CH3 sequence and the second heavy chain polypeptide comprises a second CH3 sequence.

Two CH3 sequences that comprise one or more amino acid modifications introduced in an asymmetric fashion generally results in a heterodimeric Fc, rather than a homodimer, when the two CH3 sequences dimerize. As used herein, “asymmetric amino acid modifications” refers to any modification where an amino acid at a specific position on a first CH3 sequence is different from the amino acid on a second CH3 sequence at the same position, and the first and second CH3 sequence preferentially pair to form a heterodimer, rather than a homodimer. This heterodimerization can be a result of modification of only one of the two amino acids at the same respective amino acid position on each sequence; or modification of both amino acids on each sequence at the same respective position on each of the first and second CH3 sequences. The first and second CH3 sequence of a heterodimeric Fc can comprise one or more than one asymmetric amino acid modification.

Table 2 provides the amino acid sequence of a human IgG1 Fc sequence, corresponding to amino acids 231 to 447 of a full-length human IgG1 heavy chain. The CH3 sequence comprises amino acid 341-447 of the full-length human IgG1 heavy chain.

Typically an Fc can include two contiguous heavy chain sequences (A and B) that are capable of dimerizing. In some aspects, one or both sequences of an Fc include one or more mutations or modifications at the following locations: L351, F405, Y407, T366, K392, T394, T350, S400, and/or N390, using EU numbering. In some aspects, a Fc includes a mutant sequence shown in Table 2. In some aspects, a Fc includes the mutations of Variant 1 A-B. In some aspects, a Fc includes the mutations of Variant 2 A-B. In some aspects, a Fc includes the mutations of Variant 3 A-B. In some aspects, a Fc includes the mutations of Variant 4 A-B. In some aspects, a Fc includes the mutations of Variant 5 A-B.

TABLE 2
Exemplary Fc sequence and CH3 modifications

| Human IgG1 | APELLGGPSVFLFPPKPKDTLMISRTPEVTCVVVDVSH | |
|---|---|---|
| Fc sequence | EDPEVKFNWYVDGVEVHNAKTKPREEQYNSTYRVVS | |
| 231-447 | VLTVLHQDWLNGKEYKCKVSNKALPAPIEKTISKAKG | |
| (EU- | QPREPQVYTLPPSRDELTKNQVSLTCLVKGFYPSDIAV | |
| numbering) | EWESNGQPENNYKTTPPVLDSDGSFFLYSKLTVDKSR | |
| | WQQGNVFSCSVMHEALHNHYTQKSLSLSPGK | |
| | (SEQ ID NO: 28) | |
| Variant | | |
| IgG1 Fc | | |
| sequence | | |
| (231-447) | Chain | Mutations |
| 1 | A | L351Y_F405A_Y407V |
| 1 | B | T366L_K392M_T394W |
| 2 | A | L351Y_F405A_Y407V |
| 2 | B | T366L_K392L_T394W |
| 3 | A | T350V_L351Y_F405A_Y407V |
| 3 | B | T350V_T366L_K392L_T394W |
| 4 | A | T350V_L351Y_F405A_Y407V |
| 4 | B | T350V_T366L_K392M_T394W |
| 5 | A | T350V_L351Y_S400E_F405A_Y407V |
| 5 | B | T350V_T366L_N390R_K392M_T394W |

The first and second CH3 sequences can comprise amino acid mutations as described herein, with reference to amino acids 231 to 447 of the full-length human IgG1 heavy chain. In one embodiment, the heterodimeric Fc comprises a modified CH3 domain with a first CH3 sequence having amino acid modifications at positions F405 and Y407, and a second CH3 sequence having amino acid modifications at position T394. In one embodiment, the heterodimeric Fc comprises a modified CH3 domain with a first CH3 sequence having one or more amino acid modifications selected from L351Y, F405A, and Y407V, and the second CH3 sequence having one or more amino acid modifications selected from T366L, T366I, K392L, K392M, and T394W.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, and one of the first or second CH3 sequences further comprising amino acid modifications at position Q347, and the other CH3 sequence further comprising amino acid modification at position K360. In another embodiment, a heterodimeric Fc comprises a modified CH3 domain with a first CH3 sequence having amino acid modifications at positions L351, F405 and Y407, and a second CH3 sequence having amino acid modifications at position T366, K392, and T394, one of the first or second CH3 sequences further comprising amino acid modifications at position Q347, and the other CH3 sequence further comprising amino acid modification at position K360, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394 and one of said first and second CH3 sequences further comprising amino acid modification of D399R or D399K and the other CH3 sequence comprising one or more of T411E, T411D, K409E, K409D, K392E and K392D. In another embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, one of said first and second CH3 sequences further comprises amino acid modification of D399R or D399K and the other CH3 sequence comprising one or more of T411E, T411D, K409E, K409D, K392E and K392D, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, wherein one or both of said CH3 sequences further comprise the amino acid modification of T350V.

In one embodiment, a heterodimeric Fc comprises a modified CH3 domain comprising the following amino acid modifications, where “A” represents the amino acid modifications to the first CH3 sequence, and “B” represents the amino acid modifications to the second CH3 sequence: A:L351Y_F405A_Y407V, B:T366L_K392M_T394W, A:L351Y_F405A_Y407V, B:T366L_K392L_T394W, A:T350V_L351Y_F405A_Y407V, B:T350V_T366L_K392L_T394W, A:T350V_L351Y_F405A_Y407V, B:T350V_T366L_K392M_T394W, A:T350V_L351Y_S400E_F405A_Y407V, and/or B:T350V_T366L_N390R_K392M_T394W.

The one or more asymmetric amino acid modifications can promote the formation of a heterodimeric Fc in which the heterodimeric CH3 domain has a stability that is comparable to a wild-type homodimeric CH3 domain. In an embodiment, the one or more asymmetric amino acid modifications promote the formation of a heterodimeric Fc domain in which the heterodimeric Fc domain has a stability that is comparable to a wild-type homodimeric Fc domain. In an embodiment, the one or more asymmetric amino acid modifications promote the formation of a heterodimeric Fc domain in which the heterodimeric Fc domain has a stability observed via the melting temperature (Tm) in a differential scanning calorimetry study, and where the melting temperature is within 4° C. of that observed for the corresponding symmetric wild-type homodimeric Fc domain. In some aspects, the Fc comprises one or more modifications in at least one of the C_{H3} sequences that promote the formation of a heterodimeric Fc with stability comparable to a wild-type homodimeric Fc.

In one embodiment, the stability of the CH3 domain can be assessed by measuring the melting temperature of the CH3 domain, for example by differential scanning calorimetry (DSC). Thus, in a further embodiment, the CH3 domain has a melting temperature of about 68° C. or higher. In another embodiment, the CH3 domain has a melting temperature of about 70° C. or higher. In another embodiment, the CH3 domain has a melting temperature of about 72° C. or higher. In another embodiment, the CH3 domain has a melting temperature of about 73° C. or higher. In another embodiment, the CH3 domain has a melting temperature of about 75° C. or higher. In another embodiment, the CH3 domain has a melting temperature of about 78° C. or higher. In some aspects, the dimerized C_{H3} sequences have a melting temperature (Tm) of about 68, 69, 70, 71, 72, 73, 74, 75, 76, 77, 77.5, 78, 79, 80, 81, 82, 83, 84, or 85° C. or higher.

In some embodiments, a heterodimeric Fc comprising modified CH3 sequences can be formed with a purity of at least about 75% as compared to homodimeric Fc in the expressed product. In another embodiment, the heterodimeric Fc is formed with a purity greater than about 80%. In another embodiment, the heterodimeric Fc is formed with a purity greater than about 85%. In another embodiment, the heterodimeric Fc is formed with a purity greater than about 90%. In another embodiment, the heterodimeric Fc is formed with a purity greater than about 95%. In another embodiment, the heterodimeric Fc is formed with a purity greater than about 97%. In some aspects, the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when expressed. In some aspects, the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when expressed via a single cell.

Additional methods for modifying monomeric Fc polypeptides to promote heterodimeric Fc formation are described in International Patent Publication No. WO 96/027011 (knobs into holes), in Gunasekaran et al. (Gunasekaran K. et al. (2010) J Biol Chem. 285, 19637-46, electrostatic design to achieve selective heterodimerization), in Davis et al. (Davis, J H. et al. (2010) Prot Eng Des Sel; 23(4): 195-202, strand exchange engineered domain (SEED) technology), and in Labrijn et al [Efficient generation of stable bi-specific IgG1 by controlled Fab-arm exchange. Labrijn A F, Meesters J I, de Goeij B E, van den Bremer E T, Neijssen J, van Kampen M D, Strumane K, Verploegen S, Kundu A, Gramer M J, van Berkel P H, van de Winkel J G, Schuurman J, Parren P W. Proc Natl Acad Sci USA. 2013 Mar. 26; 110(13):5145-50.

In some embodiments an isolated construct described herein comprises an antibody construct which binds an antigen; and a dimeric Fc polypeptide construct that has superior biophysical properties like stability and ease of manufacture relative to an antibody construct which does not include the same Fc polypeptide. A number of mutations in the heavy chain sequence of the Fc are known in the art for selectively altering the affinity of the antibody Fc for the different Fcgamma receptors. In some aspects, the Fc comprises one or more modifications to promote selective binding of Fc-gamma receptors.

In an exemplary embodiment, domains of a trastuzumab Fab are utilized. An exemplary construct comprises Heavy Chain A, the light chain and one of the Heavy Chain B sequences. Either wild type or mutated polypeptides can be used. In some embodiments, sequences (e.g., V_{H}, V_{L}, CDR, Fc) identical to the parent sequence are used in the conjugates of the invention. In the sequences below, Heavy Chains A and B are not wild-type, but have mutations in the CH3 domain that promote the formation of heterodimeric Fc region. Exemplary variant sequences of use in this embodiment of the invention are set forth herein below:

SEQ. ID. NO.: 1

Amino Acid Sequence—Heavy Chain a for Trastuzumab (HER_CH-A_T350V_L351Y_F405A_Y407V_no_C-term_Lys)

EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYVYPPSRDELTKNQVSLTCLVKGFYPSDIAVEWESNGQPENNYKTTPPV
LDSDGSFALVSKLTVDKSRWQQGNVFSCSVMHEALHNHYTQKSLSLSPG

SEQ. ID. NO.: 2

### Amino Acid Sequence—Light Chain for Trastuzumab

DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIK

SEQ. ID. NO.: 3

Amino Acid Sequence—Heavy Chain B with HSAdIII (HSAdIII-HET_Fc_001b_no_C-term Lys)

GVEEPQNLIKQNCELFEQLGEYKFQNALLVRYTKKVPQVSTPTLVEVSRN
LGKVGSKCCKHPEAKRMPCAEDYLSVVLNQLCVLHEKTPVSDRVTKCCTE
SLVNRRPCFSALEVDETYVPKEFNAETFTFHADICTLSEKERQIKKQTAL
VELVKHKPKATKEQLKAVMDDFAAFVEKCCKADDKETCFAEEGKKLVAAS
QAALGLAAEPKSSDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYVLPPSRDE
LTKNQVSLLCLVKGFYPSDIAVEWESNGQPENNYLTWPPVLDSDGSFFLY
SKLTVDKSRWQQGNVFSCSVMHEALHNHYTQKSLSLSPG

SEQ. ID. NO.: 4

Amino Acid Sequence—Heavy Chain B with Lys6 (Lys6-HET_Fc_001b_no_C-term_Lys)

GKKKKKKAAEPKSSDKTHTCPPCPAPELLGGPSVFLFPPKPKDTLMISRT
PEVTCVVVDVSHEDPEVKFNWYVDGVEVHNAKTKPREEQYNSTYRVVSVL
TVLHQDWLNGKEYKCKVSNKALPAPIEKTISKAKGQPREPQVYVLPPSRD
ELTKNQVSLLCLVKGFYPSDIAVEWESNGQPENNYLTWPPVLDSDGSFFL
YSKLTVDKSRWQQGNVFSCSVMHEALHNHYTQKSLSLSPG

SEQ. ID. NO.: 5

Amino Acid Sequence—Heavy Chain B with Gly-Cys (Fc_CH-B_N-term_GlyCys_T350V_T366L_K392L_T394W_no_C-term_Lys)

GCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYVLPPSRDELTKNQVSLLCL
VKGFYPSDIAVEWESNGQPENNYLTWPPVLDSDGSFFLYSKLTVDKSRWQ
QGNVFSCSVMHEALHNHYTQKSLSLSPG

SEQ. ID. NO.: 6

Amino Acid Sequence—Heavy Chain B with Cys (Fc_CH-B_N-term_Cys_T350V_T366L_K392L_T394W_no_C-term_Lys)

CDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYVLPPSRDELTKNQVSLLCLV
KGFYPSDIAVEWESNGQPENNYLTWPPVLDSDGSFFLYSKLTVDKSRWQQ
GNVFSCSVMHEALHNHYTQKSLSLSPG

SEQ. ID. NO.: 7

### Amino Acid Sequence—Heavy Chain B—Fc Region

GEPKSSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYVLPPSRDELTKNQVS
LLCLVKGFYPSDIAVEWESNGQPENNYLTWPPVLDSDGSFFLYSKLTVDK
SRWQQGNVFSCSVMHEALHNHYTQKSLSLSPG

In various embodiments, in which one or more domain of trastuzumab is incorporated into a conjugate of the invention, the conjugate binds to the HER2/neu receptor. In an exemplary embodiment, the conjugate binds to Domain IV of the HER2/neu receptor. In various embodiments, the conjugate of the invention binds to the HER2/neu receptor with a K_{d} essentially identical to the K_{d} of a structurally identical construct in which one or both of (B) or (C) is absent. In various embodiments, the K_{d} of the binding between HER2/neu and a conjugate of the invention is not less than about 95%, about 90%, about 85%, about 80%, about 75%, about 70%, about 65%, or not less than about 60% of the K_{d} of a structurally identical construcing in which one or both of (B) or (C) is absent. In various embodiments, the conjugate of the invention binds to the HER2/neu receptor of a cell with sufficient avidity that cell development is arrested at the G1 phase of the cell cycle.

In various embodiments, the conjugate contains the trastuzumab Fab and the sequence is based on the parent sequence of the approved anti-Her2 antibody trastuzumab. Trastuzumab is a recombinant IgG1 kappa, humanized monoclonal antibody that selectively binds with high affinity in a cell-based assay (Kd=5 nM) to the extracellular domain of the human epidermal growth factor receptor protein. The amino acid sequence of trastuzumab is know (See, e.g., http://www.drugbank.ca/drugs/DB00072). Produced in CHO cell cultureln an exemplary embodiment, the heavy chain CH3 domain is modified to promote the formation of a heterodimeric Fc domain with an increased stability as compared to a CH3 domain that does not include amino acid modiciations. In an exemplary embodiment, the wild type amino acid sequence is modified with the following added modifications in the heavy chain CH3 domain introduced in order to promote the formation of a heterodimer Fc domain with increased stability as compared to a CH3 domain that does not comprise amino acid mutations. At least one of Chain A and Chain B includes at least one of the following amino acid modifications: Chain A is modified with 0, 1, 2, 3, or 4 modifications, in any combination, selected from T350V, L351Y, F405A, and Y407V; and Chain B is modified with 0, 1, 2, 3, or 4 modification, in any combination, selected from T350V, T366L, K392L, and T394W. In an exemplary embodiment, Chain A is modified with T350V/L351Y/F405A/Y407V. In an exemplary embodiment, Chain B is modified with T350V/T366L/K392L/T394W. In an exemplary embodiment, Chain A is modified with: T350V/L351Y/F405A/Y407V, and Chain B is modified with: T350V/T366L/K392L/T394W.

In various embodiments, Domain III (or the polypeptide module B) is attached to the N-terminal end of the heavy chain of the antibody. In some embodiments, such as the one armed antibody, the heavy chain to which the polypeptide module B is fused could comprise of the Fc portion (CH3 and CH2 domains as well as certain residues of the hinge) of the antibody. In various embodiments, Domain III (or the polypeptide module B) is attached to the N-terminal end of the light chain of the antibody. In various embodiments, Domain III (or the polypeptide module B) is attached to the C-terminus of the heavy chain of the antibody. In various embodiments, Domain III (or the polypeptide module B) is attached to the C-terminus of the light chain of the antibody. In various embodiments, Domain III of albumin is attached via the residue at the N-terminal end. In various embodiments, Domain III is attached via the residue at the C-terminal end. In various embodiments, Domain III is a fragment of the human serum albumin sequence and the fragmentation site is at or within 1 to 10 residues of residue position 381 in human serum albumin.

In various embodiments, the invention provides a conjugate between an antibody binding specifically to HER2/neu and a therapeutic agent. The conjugate comprises, (a) a polypeptide therapeutic agent conjugation module (B) to which one or more of the therapeutic agent (A) is covalently bound. The conjugate also includes module (B), comprising a member selected from an albumin fragment, e.g., Domain I, Domain II or Domain III of albumin. The conjugate further includes (b) a polypeptide targeting module (C) covalently bound to the therapeutic agent conjugation module. Module (C) specifically binds to HER2/neu. In an exemplary embodiment, module (C) is a one-armed antibody comprising Fc region and Fv region sequences of trastuzumab, e.g., the parent sequence of trastuzumab. In an exemplary embodiment, the Fc region is a heterodimeric Fc region. In various embodiments, module (A) is diptheria toxin, and module (B) is Domain III of albumin. In an exemplary embodiment, module (B) is covalently bound to module (C) through a first amino acid at the C-terminus of module (B) and a second amino acid at the N-terminus of said module (C). In various embodiment, the converse is true and module (B) is covalently bound to module (C) through a first amino acid at the N-terminus of said module (B) and a second amino acid at the C-terminus of module (C). As will be appreciated by those of skill in the art, amino acids may be added, removed or substituted at one or both of the C- and N-terminus to effect the conjugation of module (B) and module (C). In an exemplary embodiment, 1, 2, 3, 4, 5, 6, 7 8, 9, 10 or more amino acids are removed from the C- and or N-terminus of either or both module (B) and module (C). In various embodiments, this number of amino acids is added to either or both the C- and N-terminus of either or both module (B) and module (C). In an exemplary embodiment, 1, 2, 3, 4, 5, 6, 7 8, 9, 10 or more amino acids are substituted relevant to the parent sequence at the C- and or N-terminus of either or both module (B) and module (C). In an exemplary embodiment, the numbering set forth above begins at the C- and/or N-terminus of module (b) and/or module (C) and progresses inwards through consecutive amino acids.

In various embodiments, the first and second amino acids are bound through a covalent bond or through a linker interposed between, and bound to both of, the first amino acid and the second amino acid.

In an exemplary embodiment, the invention provides a conjugate comprising, Heavy Chain A (SEQ. ID. NO.: 12), Heavy Chain B (SEQ. ID. NO.: 14), and light chain (SEQ. ID. NO.: 16).

As will be appreciated by those of skill in the art, the various polypeptide components of the conjugates described above, e.g., Heavy Chain B, Heavy Chain A, Fab, etc., either conform to the parent polypeptide sequence or, optionally, they include 1, 2, 3, 4, 5, 6, 7, 8, 9, 10 or more additions, deletions or substitutions. In various embodiments, the module is at least 90%, at least 92%, at least 94%, at least 96%, at least 98% or at least 99% homologous to the parent polypeptide sequence.

### Generation of Novel Polypeptides

The polypeptide component of the conjugates of the invention may be derived from a primary sequence of a native polypeptide, or may be engineered using any of the many means known to those of skill in the art. Such engineered polypeptides can be designed and/or selected because of enhanced or novel properties as compared with the native polypeptide. For example, polypeptides may be engineered to have increased enzyme reaction rates, increased or decreased binding affinity to a substrate or ligand, increased or decreased binding affinity to a receptor, altered specificity for a substrate, ligand, receptor or other binding partner, increased or decreased stability in vitro and/or in vivo, or increased or decreased immunogenicity in an animal.

The polypeptide components of the conjugates of the invention may be mutated to enhance a desired biological activity or function, to diminish an undesirable property of the polypeptide, and/or to add novel activities or functions to the polypeptide. “Rational polypeptide design” may be used to generate such altered polypeptides. Once the amino acid sequence and structure of the polypeptide is known and a desired mutation planned, the mutations can be made most conveniently to the corresponding nucleic acid codon which encodes the amino acid residue that is desired to be mutated. One of skill in the art can easily determine how the nucleic acid sequence should be altered based on the universal genetic code, and knowledge of codon preferences in the expression system of choice. A mutation in a codon may be made to change the amino acid residue that will be polymerized into the polypeptide during translation. Alternatively, a codon may be mutated so that the corresponding encoded amino acid residue is the same, but the codon choice is better suited to the desired polypeptide expression system. For example, cys-residues may be replaced with other amino acids to remove disulfide bonds from the mature polypeptide, catalytic domains may be mutated to alter biological activity, and in general, isoforms of the polypeptide can be engineered. Such mutations can be point mutations, deletions, insertions and truncations, among others.

Techniques to mutate specific amino acids in a polypeptide are well known in the art. The technique of site-directed mutagenesis, discussed above, is well suited for the directed mutation of codons. The oligonucleotide-mediated mutagenesis method is also discussed in detail in Sambrook et al. (2001, Molecular Cloning: A Laboratory Manual, Cold Spring Harbor Laboratory, New York, starting at page 15.51). Systematic deletions, insertions and truncations can be made using linker insertion mutagenesis, digestion with nuclease Bal31, and linker-scanning mutagenesis, among other method well known to those in the art (Sambrook et al., 2001, Molecular Cloning: A Laboratory Manual, Cold Spring Harbor Laboratory, New York).

Rational polypeptide design has been successfully used to increase the stability of enzymes with respect to thermoinactivation and oxidation. For example, the stability of an enzyme was improved by removal of asparagine residues in α-amylase (Declerck et al., 2000, J. Mol. Biol. 301:1041-1057), the introduction of more rigid structural elements such as proline into α-amylase (Igarashi et al., 1999, Biosci. Biotechnol. Biochem. 63:1535-1540) and D-xylose isomerase (Zhu et al., 1999, polypeptide Eng. 12:635-638). Further, the introduction of additional hydrophobic contacts stabilized 3-isopropylmalate dehydrogenase (Akanuma et al., 1999, Eur. J. Biochem. 260:499-504) and formate dehydrogenase obtained from Pseudomonas sp. (Rojkova et al., 1999, FEBS Lett 445:183-188). The mechanisms behind the stabilizing effect of these mutations is generally applicable to many polypeptides. These and similar mutations are contemplated to be useful with respect to the polypeptides components of the conjugates of the present invention.

Novel polypeptides useful in the methods of the invention may be generated using techniques that introduce random mutations in the coding sequence of the nucleic acid. The nucleic acid is then expressed in a desired expression system, and the resulting polypeptide is assessed for properties of interest. Techniques to introduce random mutations into DNA sequences are well known in the art, and include PCR mutagenesis, saturation mutagenesis, and degenerate oligonucleotide approaches. See Sambrook and Russell (2001, Molecular Cloning, A Laboratory Approach, Cold Spring Harbor Press, Cold Spring Harbor, N.Y.) and Ausubel et al. (2002, Current Protocols in Molecular Biology, John Wiley & Sons, NY).

In PCR mutagenesis, reduced Taq polymerase fidelity is used to introduce random mutations into a cloned fragment of DNA (Leung et al., 1989, Technique 1:11-15). This is a very powerful and relatively rapid method of introducing random mutations into a DNA sequence. The DNA region to be mutagenized is amplified using the polymerase chain reaction (PCR) under conditions that reduce the fidelity of DNA synthesis by Taq DNA polymerase, e.g., by using an altered dGTP/dATP ratio and by adding Mn²⁺ to the PCR reaction. The pool of amplified DNA fragments are inserted into appropriate cloning vectors to provide random mutant libraries.

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complementary DNA strand. The mutation frequency can be modulated by modulating the severity of the treatment, and essentially all possible base substitutions can be obtained. Because this procedure does not involve a genetic selection for mutant fragments, both neutral substitutions as well as those that alter function, are obtained. The distribution of point mutations is not biased toward conserved sequence elements.

A library of nucleic acid homologs can also be generated from a set of degenerate oligonucleotide sequences. Chemical synthesis of a degenerate oligonucleotide sequences can be carried out in an automatic DNA synthesizer, and the synthetic genes may then be ligated into an appropriate expression vector. The synthesis of degenerate oligonucleotides is known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other polypeptides (see, for example, Scott et al. (1990) Science 249:386-390; Roberts et al. (1992) PNAS 89:2429-2433; Devlin et al. (1990) Science 249:404-406; Cwirla et al. (1990) PNAS 87:6378-6382; as well as U.S. Pat. Nos. 5,223,409, 5,198,346, and 5,096,815).

Peptides useful in the conjugates of the invention may also be generated using “directed evolution” techniques. In contrast to site directed mutagenesis techniques where knowledge of the structure of the polypeptide is required, there now exist strategies to generate libraries of mutations from which to obtain polypeptides with improved properties without knowledge of the structural features of the polypeptide. These strategies are generally known as “directed evolution” technologies and are different from traditional random mutagenesis procedures in that they involve subjecting the nucleic acid sequence encoding the polypeptide of interest to recursive rounds of mutation, screening and amplification.

In some “directed evolution” techniques, the diversity in the nucleic acids obtained is generated by mutation methods that randomly create point mutations in the nucleic acid sequence. The point mutation techniques include, but are not limited to, “error-prone PCR™” (Caldwell and Joyce, 1994; PCR Methods Appl. 2:28-33; and Ke and Madison, 1997, Nucleic Acids Res. 25:3371-3372), repeated oligonucleotide-directed mutagenesis (Reidhaar-Olson et al., 1991, Methods Enzymol. 208:564-586), and any of the aforementioned methods of random mutagenesis.

Another method of creating diversity upon which directed evolution can act is the use of mutator genes. The nucleic acid of interest is cultured in a mutator cell strain the genome of which typically encodes defective DNA repair genes (U.S. Pat. No. 6,365,410; Selifonova et al., 2001, Appl. Environ. Microbiol. 67:3645-3649; Long-McGie et al., 2000, Biotech. Bioeng. 68:121-125; see, Genencor International Inc, Palo Alto Calif.).

Achieving diversity using directed evolution techniques may also be accomplished using saturation mutagenesis along with degenerate primers (Gene Site Saturation Mutagenesis™, Diversa Corp., San Diego, Calif.). In this type of saturation mutagenesis, degenerate primers designed to cover the length of the nucleic acid sequence to be diversified are used to prime the polymerase in PCR reactions. In this manner, each codon of a coding sequence for an amino acid may be mutated to encode each of the remaining common nineteen amino acids. This technique may also be used to introduce mutations, deletions and insertions to specific regions of a nucleic acid coding sequence while leaving the rest of the nucleic acid molecule untouched. Procedures for the gene saturation technique are well known in the art, and can be found in U.S. Pat. No. 6,171,820.

Novel polypeptides useful in the conjugates of the invention may also be generated using the techniques of gene-shuffling, motif-shuffling, exon-shuffling, and/or codon-shuffling (collectively referred to as “DNA shuffling”). DNA shuffling techniques are may be employed to modulate the activities of polypeptides useful in the invention and may be used to generate polypeptides having altered activity. See, generally, U.S. Pat. Nos. 5,605,793; 5,811,238; 5,830,721; 5,834,252; and 5,837,458, and Stemmer et al. (1994, Nature 370(6488):389-391); Crameri et al. (1998, Nature 391 (6664):288-291); Zhang et al. (1997, Proc. Natl. Acad. Sci. USA 94(9):4504-4509); Stemmer et al. (1994, Proc. Natl. Acad. Sci USA 91(22):10747-10751), Patten et al. (1997, Curr. Opinion Biotechnol. 8:724-33); Harayama, (1998, Trends Biotechnol. 16(2):76-82); Hansson, et al., (1999, J Mol. Biol. 287:265-76); and Lorenzo and Blasco (1998, Biotechniques 24(2):308-13) (each of these patents are hereby incorporated by reference in its entirety).

DNA shuffling involves the assembly of two or more DNA segments by homologous or site-specific recombination to generate variation in the polynucleotide sequence. DNA shuffling has been used to generate novel variations of human immunodeficiency virus type 1 proteins (Pekrun et al., 2002, J. Virol. 76(6):2924-35), triazine hydrolases (Raillard et al. 2001, Chem Biol 8(9):891-898), murine leukemia virus (MLV) proteins (Powell et al. 2000, Nat Biotechnol 18(12):1279-1282), and indoleglycerol phosphate synthase (Merz et al. 2000, Biochemistry 39(5): 880-889).

The technique of DNA shuffling was developed to generate biomolecular diversity by mimicking natural recombination by allowing in vitro homologous recombination of DNA (Stemmler, 1994, Nature 370: 389-391; and Stemmler, 1994, PNAS 91:10747-10751). Generally, in this method a population of related genes is fragmented and subjected to recursive cycles of denaturation, rehybridization, followed by the extension of the 5′ overhangs by Taq polymerase. With each cycle, the length of the fragments increases, and DNA recombination occurs when fragments originating from different genes hybridize to each other. The initial fragmentation of the DNA is usually accomplished by nuclease digestion, typically using DNase (see Stemmler references, above), but may also be accomplished by interrupted PCR synthesis (U.S. Pat. No. 5,965,408, incorporated herein by reference in its entirety; see, Diversa Corp., San Diego, Calif.). DNA shuffling methods have advantages over random point mutation methods in that direct recombination of beneficial mutations generated by each round of shuffling is achieved and there is therefore a self selection for improved phenotypes of polypeptides.

The techniques of DNA shuffling are well known to those in art. Detailed explanations of such technology is found in Stemmler, 1994, Nature 370:389-391 and Stemmler, 1994, PNAS 91:10747-10751. The DNA shuffling technique is also described in U.S. Pat. Nos. 6,180,406, 6,165,793, 6,132,970, 6,117,679, 6,096,548, 5,837,458, 5,834,252, 5,830,721, 5,811,238, and 5,605,793 (all of which are incorporated by reference herein in their entirety).

The art also provides even more recent modifications of the basic technique of DNA shuffling. In one example, exon shuffling, exons or combinations of exons that encode specific domains of polypeptides are amplified using chimeric oligonucleotides. The amplified molecules are then recombined by self-priming PCR assembly (Kolkman and Stemmler, 2001, Nat. Biotech. 19:423-428). In another example, using the technique of random chimeragenesis on transient templates (RACHITT) library construction, single stranded parental DNA fragments are annealed onto a full-length single-stranded template (Coco et al., 2001, Nat. Biotechnol. 19:354-359). In yet another example, staggered extension process (StEP), thermocycling with very abbreviated annealing/extension cycles is employed to repeatedly interrupt DNA polymerization from flanking primers (Zhao et al., 1998, Nat. Biotechnol. 16:258-261). In the technique known as CLERY, in vitro family shuffling is combined with in vivo homologous recombination in yeast (Abecassis et al., 2000, Nucleic Acids Res. 28:E88). To maximize intergenic recombination, single stranded DNA from complementary strands of each of the nucleic acids is digested with DNase and annealed (Kikuchi et al., 2000, Gene 243:133-137). The blunt ends of two truncated nucleic acids of variable lengths that are linked by a cleavable sequence are then ligated to generate gene fusion without homologous recombination (Sieber et al., 2001, Nat Biotechnol. 19:456-460; Lutz et al., 2001, Nucleic Acids Res. 29:E16; Ostermeier et al., 1999, Nat. Biotechnol. 17:1205-1209; Lutz and Benkovic, 2000, Curr. Opin. Biotechnol. 11:319-324). Recombination between nucleic acids with little sequence homology in common has also been enhanced using exonuclease-mediated blunt-ending of DNA fragments and ligating the fragments together to recombine them (U.S. Pat. No. 6,361,974, incorporated herein by reference in its entirety). The invention contemplates the use of each and every variation described above as a means of enhancing the biological properties of any of the polypeptides and/or enzymes useful in the methods of the invention.

Following each recursive round of “evolution,” the desired polypeptides expressed by mutated genes are screened for characteristics of interest. The “candidate” genes are then amplified and pooled for the next round of DNA shuffling. The screening procedure used is highly dependant on the polypeptide that is being “evolved” and the characteristic of interest. Characteristics such as polypeptide stability, biological activity, antigenicity, among others can be selected using procedures that are well known in the art. Individual assays for the biological activity of preferred polypeptides useful in the methods of the invention are described elsewhere herein.

It will be appreciated by the skilled artisan that the above techniques of mutation and selection can be combined with each other and with additional procedures to generate the best possible polypeptide molecule useful in the methods of the invention. Thus, the invention is not limited to any one method for the generation of polypeptides, and should be construed to encompass any and all of the methodology described herein. For example, a procedure for introducing point mutations into a nucleic acid sequence may be performed initially, followed by recursive rounds of DNA shuffling, selection and amplification. The initial introduction of point mutations may be used to introduce diversity into a gene population where it is lacking, and the following round of DNA shuffling and screening will select and recombine advantageous point mutations.

In various embodiments, the fusion between the antibody polypeptide or albumin Domain III polypeptide happens via the residues at the N and C-terminal ends of the two polypeptide chains. In various embodiments, the fusion to the antibody polypeptide happens to one or more of the multiple polypeptide chains present in the heteromultimeric antibody molecule. In various embodiments, the fusion between the antibody and albumin Domain III polypeptides involves a C-terminal to N-terminal genetic fusion of the two polypeptide chains. In another embodiment, the order of polypeptides is reversed. In various embodiments, the fusion between the antibody and albumin Domain III polypeptides involves a chemical functionalization and fusion of the two polypeptide chains. In various embodiments, the chemical functionalization and subsequent chemical fusion reaction involves in the N-terminal ends of the two polypeptide chains. In various embodiments, the chemical fusion involves the introduction of a Cys residue at the N-terminal end of the polypeptide. In various embodiments, the chemical fusion could involve a biomimetic transamination step at the N-terminus of the polypeptide chain (e.g. Witus LS and Francis MB (2010) Site-specific protein bioconjugation via a pyridoxal 5′-phospahte-mediated N-terminal transamination reaction. Curr Protoc Chem Biol 2, 125-134).

### Expression of Polypeptides

In general to express a polypeptide from a nucleic acid encoding it, the nucleic acid must be incorporated into an expression cassette, comprising a promoter element, a terminator element, and the coding sequence of the polypeptide operably linked between the two. The expression cassette is then operably linked into a vector. Toward this end, adapters or linkers may be employed to join the nucleotide fragments or other manipulations may be involved to provide for convenient restriction sites, removal of superfluous nucleotides, removal of restriction sites, or the like. For this purpose, in vitro mutagenesis, primer repair, restriction, annealing, resubstitutions, e.g., transitions and transversions, may be involved. A shuttle vector has the genetic elements necessary for replication in a cell. Some vectors may be replicated only in prokaryotes, or may be replicated in both prokaryotes and eukaryotes. Such a plasmid expression vector will be maintained in one or more replication systems, preferably two replication systems, allowing for stable maintenance within a yeast host cell for expression purposes, and within a prokaryotic host for cloning purposes. Many vectors with diverse characteristics are now available commercially. Vectors are usually plasmids or phages, but may also be cosmids or mini-chromosomes. Conveniently, many commercially available vectors will have the promoter and terminator of the expression cassette already present, and a multi-linker site where the coding sequence for the polypeptide of interest can be inserted. The shuttle vector containing the expression cassette is then transformed in E. coli where it is replicated during cell division to generate a preparation of vector that is sufficient to transform the host cells of the chosen expression system. The above methodology is well know to those in the art, and protocols by which to accomplish can be found Sambrook et al. (2001, Molecular Cloning: A Laboratory Manual, Cold Spring Harbor Laboratory, New York).

The vector, once purified from the cells in which it is amplified, is then transformed into the cells of the expression system. The protocol for transformation depended on the kind of the cell and the nature of the vector. Transformants are grown in an appropriate nutrient medium, and, where appropriate, maintained under selective pressure to insure retention of endogenous DNA. Where expression is inducible, growth can be permitted of the yeast host to yield a high density of cells, and then expression is induced. The secreted, mature heterologous polypeptide can be harvested by any conventional means, and purified by chromatography, electrophoresis, dialysis, solvent-solvent extraction, and the like.

The techniques of molecular cloning are well-known in the art. Further, techniques for the procedures of molecular cloning can be found in Sambrook et al. (2001, Molecular Cloning: A Laboratory Manual, Cold Spring Harbor Laboratory Press, Cold Spring Harbor, N.Y.); Glover et al., (1985, DNA Cloning: A Practical Approach, Volumes I and II); Gait et al., (1985, Oligonucleotide Synthesis); Hames and Higgins (1985, Nucleic Acid Hybridization); Hames and Higgins (1984, Transcription And Translation); Freshney et al., (1986, Animal Cell Culture); Perbal, (1986, Immobilized Cells And Enzymes, IRL Press); Perbal (1984, A Practical Guide To Molecular Cloning); Ausubel et al. (2002, Current Protocols in Molecular Biology, John Wiley & Sons, Inc.).

### Purification of Polypeptides and Conjugates

If the modified polypeptide is produced intracellularly or secreted, as a first step, the particulate debris, either host cells, lysed fragments, is removed, for example, by centrifugation or ultrafiltration; optionally, the protein may be concentrated with a commercially available protein concentration filter, followed by separating the polypeptide variant from other impurities by one or more steps selected from immunoaffinity chromatography, ion-exchange column fractionation (e.g., on diethylaminoethyl (DEAE) or matrices containing carboxymethyl or sulfopropyl groups), chromatography on Blue-Sepharose, CM Blue-Sepharose, MONO-Q, MONO-S, lentil lectin-Sepharose, WGA-Sepharose, Con A-Sepharose, Ether Toyopearl, Butyl Toyopearl, Phenyl Toyopearl, or protein A Sepharose, SDS-PAGE chromatography, silica chromatography, chromatofocusing, reverse phase HPLC (RP-HPLC), gel filtration using, e.g., Sephadex molecular sieve or size-exclusion chromatography, chromatography on columns that selectively bind the polypeptide, and ethanol, pH or ammonium sulfate precipitation, membrane filtration and various techniques.

Peptides produced in culture are usually isolated by initial extraction from cells, enzymes, etc., followed by one or more concentration, salting-out, aqueous ion-exchange, or size-exclusion chromatography steps. Additionally, the modified glycoprotein may be purified by affinity chromatography. Then, HPLC may be employed for final purification steps.

A protease inhibitor, e.g., phenylmethylsulfonylfluoride (PMSF) may be included in any of the foregoing steps to inhibit proteolysis and antibiotics may be included to prevent the growth of adventitious contaminants.

Within another embodiment, supernatants from systems which produce the modified polypeptide of the invention are first concentrated using a commercially available protein concentration filter, for example, an Amicon or Millipore Pellicon ultrafiltration unit. Following the concentration step, the concentrate may be applied to a suitable purification matrix. For example, a suitable affinity matrix may comprise a ligand for the polypeptide, a lectin or antibody molecule bound to a suitable support. Alternatively, an anion-exchange resin may be employed, for example, a matrix or substrate having pendant DEAE groups. Suitable matrices include acrylamide, agarose, dextran, cellulose, or other types commonly employed in protein purification. Alternatively, a cation-exchange step may be employed. Suitable cation exchangers include various insoluble matrices comprising sulfopropyl or carboxymethyl groups. Sulfopropyl groups are particularly preferred.

Then, one or more RP-HPLC steps employing hydrophobic RP-HPLC media, e.g., silica gel having pendant methyl or other aliphatic groups, may be employed to further purify a polypeptide variant composition. Some or all of the foregoing purification steps, in various combinations, can also be employed to provide a homogeneous modified glycoprotein.

The polypeptide of use in the conjugates of the invention resulting from a large-scale fermentation may be purified by methods analogous to those disclosed by Urdal et al., J. Chromatog. 296:171 (1984).

### Pharmaceutical Compositions

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980).

Pharmaceutical compositions or formulations of the present invention include combinations of a conjugate of the invention, a chemotherapeutic agent, and one or more pharmaceutically acceptable carrier, glidant, diluent, or excipient.

A conjugate of the invention and chemotherapeutic agents may exist in unsolvated as well as solvated forms with pharmaceutically acceptable solvents such as water, ethanol, and the like, and it is intended that the invention embrace both solvated and unsolvated forms.

A conjugate of the invention and chemotherapeutic agents may also exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Pharmaceutical compositions encompass both the bulk composition and individual dosage units comprised of more than one (e.g., two) pharmaceutically active agents including a conjugate of the invention and a chemotherapeutic agent selected from the lists of the additional agents described herein, along with any pharmaceutically inactive excipients, diluents, carriers, or glidants. The bulk composition and each individual dosage unit can contain fixed amounts of the aforesaid pharmaceutically active agents. The bulk composition is material that has not yet been formed into individual dosage units. An illustrative dosage unit is an oral dosage unit such as tablets, pills, capsules, and the like. Similarly, the herein-described method of treating a patient by administering a pharmaceutical composition of the present invention is also intended to encompass the administration of the bulk composition and individual dosage units.

Pharmaceutical compositions also embrace isotopically-labeled compounds of the present invention which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine and iodine such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically-labeled compounds of the present invention (e.g., those labeled with ³H and ¹⁴C) are useful in compound and/or substrate tissue distribution assays. Tritiated (³H) and carbon-14 (¹⁴C) isotopes are useful for their ease of preparation and detectability. Further, substitution with heavier isotopes such as deuterium (²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

A conjugate of the invention and chemotherapeutic agents may be formulated in accordance with standard pharmaceutical practice for use in a therapeutic combination for therapeutic treatment (including prophylactic treatment) of hyperproliferative disorders in mammals including humans. The invention provides a pharmaceutical composition comprising a conjugate of the invention in association with one or more pharmaceutically acceptable carrier, glidant, diluent, or excipient.

Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration with pharmaceutically acceptable diluents, carriers, excipients or stabilizers (Remington's Pharmaceutical Sciences (1995) 18th edition, Mack Publ. Co., Easton, Pa.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8.

The pharmaceutical formulation is preferably sterile. In particular, formulations to be used for in vivo administration must be sterile. Such sterilization is readily accomplished by filtration through sterile filtration membranes.

The pharmaceutical formulation ordinarily can be stored as a solid composition, a lyophilized formulation or as an aqueous solution.

The pharmaceutical formulations of the invention will be dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

Dosages and administration protocols for the treatment of cancers using the foregoing methods will vary with the method and the target cancer, and will generally depend on a number of other factors appreciated in the art. As a general proposition, the initial pharmaceutically effective amount of a conjugate of the invention administered per dose will be in the range of about 0.01-100 mg/kg, namely about 0.1 to 20 mg/kg of patient body weight per day, with the typical initial range of compound used being 0.3 to 15 mg/kg/day.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, ethanol, or benzylalcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, including Tween 80, PLURONICS™ or polyethylene glycol (PEG), including PEG400. The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition, (1995) Mack Publ. Co., Easton, Pa.

The pharmaceutical formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences 18^{th} Ed. (1995) Mack Publishing Co., Easton, Pa. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a chemotherapeutic agent suitable for oral administration may be prepared as discrete units such as pills, hard or soft e.g., gelatin capsules, cachets, troches, lozenges, aqueous or oil suspensions, dispersible powders or granules, emulsions, syrups or elixirs each containing a predetermined amount of a compound of a conjugate of the invention and/or a chemotherapeutic agent. Such formulations may be prepared according to any method known to the art for the manufacture of pharmaceutical compositions and such compositions may contain one or more agents including sweetening agents, flavoring agents, coloring agents and preserving agents, in order to provide a palatable preparation. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablet excipients of a pharmaceutical formulation of the invention may include: Filler (or diluent) to increase the bulk volume of the powdered drug making up the tablet; Disintegrants to encourage the tablet to break down into small fragments, ideally individual drug particles, when it is ingested and promote the rapid dissolution and absorption of drug; Binder to ensure that granules and tablets can be formed with the required mechanical strength and hold a tablet together after it has been compressed, preventing it from breaking down into its component powders during packaging, shipping and routine handling; Glidant to improve the flowability of the powder making up the tablet during production; Lubricant to ensure that the tableting powder does not adhere to the equipment used to press the tablet during manufacture. They improve the flow of the powder mixes through the presses and minimize friction and breakage as the finished tablets are ejected from the equipment; Antiadherent with function similar to that of the glidant, reducing adhesion between the powder making up the tablet and the machine that is used to punch out the shape of the tablet during manufacture; Flavor incorporated into tablets to give them a more pleasant taste or to mask an unpleasant one, and Colorant to aid identification and patient compliance.

Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions of this invention may be constituted from known ingredients in a known manner, including a mixture of at least one emulsifier with a fat or an oil, or with both a fat and an oil. Preferably, a hydrophilic emulsifier is included together with a lipophilic emulsifier which acts as a stabilizer. Together, the emulsifier(s) with or without stabilizer(s) make up an emulsifying wax, and the wax together with the oil and fat comprise an emulsifying ointment base which forms the oily dispersed phase of cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions of the pharmaceutical formulations of the invention contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

Pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may be a solution or a suspension in a non-toxic parenterally acceptable diluent or solvent, such as a solution in 1,3-butanediol or prepared from a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 .mu.g of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

In an exemplary embodiment, the conjugate of the invention is formulated for subcutaneous administration and the formulating includes hyalouronidase (See, e.g., U.S. Pat. Pub. No 20110044977).

### Combination Therapy

A conjugate of the invention may be employed in combination with other chemotherapeutic agents for the treatment of a hyperproliferative disease or disorder, including tumors, cancers, and neoplastic tissue, along with pre-malignant and non-neoplastic or non-malignant hyperproliferative disorders. In certain embodiments, a conjugate of the invention is combined in a pharmaceutical combination formulation, or dosing regimen as combination therapy, with a second compound that has anti-hyperproliferative properties or that is useful for treating the hyperproliferative disorder. The second compound of the pharmaceutical combination formulation or dosing regimen preferably has complementary activities to a conjugate of the invention, and such that they do not adversely affect each other. Such compounds are suitably present in combination in amounts that are effective for the purpose intended. In one embodiment, a composition of this invention comprises a conjugate of the invention in combination with a chemotherapeutic agent such as described herein.

Therapeutic combinations of the invention include a formulation, dosing regimen, or other course of treatment comprising the administration of a conjugate of the invention, and a chemotherapeutic agent selected from a HER2 dimerization inhibitor antibody, an anti-VEGF antibody, 5-FU, carboplatin, lapatinib, ABT-869, and docetaxel, as a combined preparation for separate, simultaneous or sequential use in the treatment of a hyperproliferative disorder.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other chemotherapeutic agents or treatments.

In a particular embodiment of anti-cancer therapy, a conjugate of the invention may be combined with a chemotherapeutic agent, including hormonal or antibody agents such as those described herein, as well as combined with surgical therapy and radiotherapy. The amounts of a conjugate of the invention and the other pharmaceutically active chemotherapeutic agent(s) and the relative timings of administration will be selected in order to achieve the desired combined therapeutic effect.

### Administration of Pharmaceutical Compositions

The compounds of the invention may be administered by any route appropriate to the condition to be treated. Suitable routes include oral, parenteral (including subcutaneous, intramuscular, intravenous, intraarterial, inhalation, intradermal, intrathecal, epidural, and infusion techniques), transdermal, rectal, nasal, topical (including buccal and sublingual), vaginal, intraperitoneal, intrapulmonary and intranasal. Topical administration can also involve the use of transdermal administration such as transdermal patches or iontophoresis devices. Formulation of drugs is discussed in Remington's Pharmaceutical Sciences, 18.sup.th Ed., (1995) Mack Publishing Co., Easton, Pa. Other examples of drug formulations can be found in Liberman, H. A. and Lachman, L., Eds., Pharmaceutical Dosage Forms, Marcel Decker, Vol 3, 2.sup.nd Ed., New York, N.Y. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound is administered orally, it may be formulated as a pill, capsule, tablet, etc. with a pharmaceutically acceptable carrier, glidant, or excipient. Where the compound is administered parenterally, it may be formulated with a pharmaceutically acceptable parenteral vehicle or diluent, and in a unit dosage injectable form, as detailed below.

A dose of a conjugate of the invention to treat human patients may range from about 100 mg to about 500 mg. The dose of a conjugate of the invention may be administered once every six weeks, once every three weeks, weekly, or more frequently, depending on the pharmacokinetic (PK) and pharmacodynamic (PD) properties, including absorption, distribution, metabolism, and excretion. A dose of the chemotherapeutic agent, used in combination with a conjugate of the invention, may range from about 10 mg to about 1000 mg. The chemotherapeutic agent may be administered once every six weeks, once every three weeks, weekly, or more frequently, such as once or twice per day. In addition, toxicity factors may influence the dosage and administration regimen. When administered orally, the pill, capsule, or tablet may be ingested daily or less frequently for a specified period of time. The regimen may be repeated for a number of cycles of therapy.

### Methods

In an exemplary embodiment, the invention provides a method of treating a disease in a subject in need of such treatment. An exemplary method comprises: administering to the subject a therapeutically effective amount of a conjugate of the invention.

Therapeutic combinations of: (1) a conjugate of the invention and (2) a chemotherapeutic agent are useful for treating diseases, conditions and/or disorders including, but not limited to, those characterized by activation of the HER2 pathway. Accordingly, another aspect of this invention includes methods of treating diseases or conditions that can be treated by targeting HER2 or the VEGFR receptor 1. Therapeutic combinations of: (1) a conjugate of the invention and (2) a chemotherapeutic agent may be employed for the treatment of a hyperproliferative disease or disorder, including tumors, cancers, and neoplastic tissue, along with pre-malignant and non-neoplastic or non-malignant hyperproliferative disorders.

In various embodiments, the invention provides a method of diagnosing a disease by detecting a disease marker in a sample, said method comprising: contacting said sample with a conjugate between an antibody and a detectable agent, said conjugate comprising: (a) a polypeptide detectable agent conjugation module (B) to which one or more of detectable agent (A) molecule is covalently bound; and (b) a polypeptide targeting module (C) covalently bound to the detectable agent conjugation module, and determining whether the antibody binds to the marker in the sample by detecting the detectable agent.

Diseases that can be detected and treated by methods of the invention include, without limitation, Cancers which can be treated according to the methods of this invention include, but are not limited to, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

The following examples are provided to illustrate certain embodiments of the invention. The invention is not limited to these examples and the full scope of the invention is reflected in the claims appended hereto.

### EXAMPLES

### Example 1

### Preparation and Expression of Constructs

A number of constructs were prepared as described in Table A1 below. Constructs containing anti-Her2 Fab were based on the sequence of the wild-type trastuzumab antibody (Table A2, SEQ ID NO:8 for Heavy Chain amino acid sequence, SEQ ID NO:9 for Light Chain sequence) with the following added modifications in the heavy chain CH3 domain introduced in order to promote the formation of a heterodimer Fc domain with increased stability as compared to a CH3 domain that does not comprise amino acid mutations:

Chain A: T350V/L351Y/F405A/Y407V, and

Chain B: T350V/T366L/K392L/T394W

The amino acid positions for antibody sequences referenced herein are numbered using the EU numbering system. All constructs containing the Human Serum Albumin (HSA) domain III were based on the sequence of recombinant fragment derived wild-type human HSA (see Table A2, SEQ ID NO:10, SEQ ID NO:11 is an exemplary DNA seq) as described in Dockal et al. (J Biol Chem (1999) 274, 29303-10). Isolated HSA Domain III (v9992) was purchased from Albumin Bioscience (cat. no. 9903, see Table A2, SEQ ID NO:24).

TABLE A1
List of constructs

| | | SEQ ID NOs: | SEQ ID NOs: |
|---|---|---|---|
| Variant | Description | (polypeptide) | (DNA) |
| 5110 | polypeptide-targeting module | Heavy chain A: | Heavy chain A: |
| | fused to polypeptide | SEQ ID NO: 12 | SEQ ID NO: 13 |
| | therapeutic agent conjugation | Heavy chain B: | Heavy chain B: |
| | module (antibody-domain III | SEQ ID NO: 14 | SEQ ID NO: 15 |
| | fusion protein)-Heterodimeric | Light chain: | Light chain: |
| | Fc fused to Her2 binding | SEQ ID NO: 16 | SEQ ID NO: 17 |
| | Fab on one heavy chain and | | |
| | domain III of albumin on | | |
| | the other heavy chain | | |
| | (see FIG. 17 for schematic | | |
| | representation) | | |
| 1040 | Control polypeptide targeting | Heavy chain A: | Heavy chain A: |
| | module-Heterodimeric Fc | SEQ ID NO: 18 | SEQ ID NO: 19 |
| | fused to Her2 binding Fab on | Heavy chain B: | Heavy chain B: |
| | one heavy chain (see FIG. 17 | SEQ ID NO: 20 | SEQ ID NO: 21 |
| | for schematic representation) | Light chain: | Light chain: |
| | | SEQ ID NO: 16 | SEQ ID NO: 17 |
| 6265 | polypeptide targeting module- | Heavy chain A: | Heavy chain A: |
| | Heterodimeric Fc fused to | SEQ ID NO: 12 | SEQ ID NO: 13 |
| | Her2 binding Fab on one | Heavy chain B: | Heavy chain B: |
| | heavy chain and N-terminal | SEQ ID NO: 22 | SEQ ID NO: 23 |
| | Cys on the other chain | Light chain: | Light chain: |
| | | SEQ ID NO: 16 | SEQ ID NO: 17 |
| 9992 | polypeptide-therapeutic agent | SEQ ID NO: 24 | SEQ ID NO: 25 |
| | conjugation module- | | |
| | Recombinant domain III of | | |
| | human serum albumin (see | | |
| | FIG. 17 for schematic | | |
| | representation) | | |
| 506 | Control-trastuzumab | Heavy chain: | Heavy chain: |
| | | SEQ ID NO: 8 | SEQ ID NO: 26 |
| | | Light chain: | Light chain: |
| | | SEQ ID NO: 9 | SEQ ID NO: 27 |

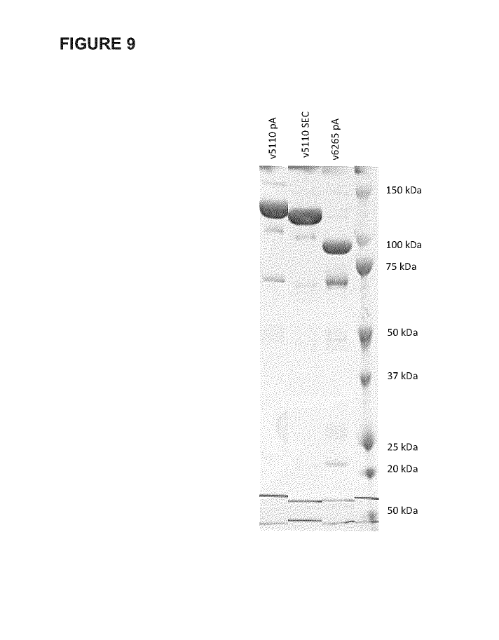

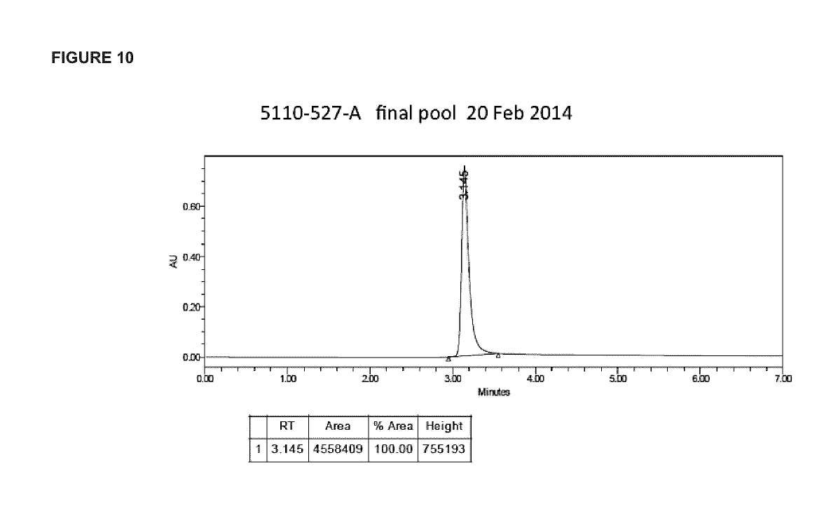

The antibody variants and controls were cloned and expressed as follows. DNA was produced by gene synthesis using standard methods. The final DNA was sub-cloned into the vector pTT5 (see International Patent Publication No. WO 2009/137911). Expression was carried out in either 2 mL or 3 L CHO 3E7 cells. CHO cells were transfected in exponential growth phase (1.5 to 2 million cells/mL) with aqueous 1 mg/mL 25 kDa polyethylenimine (PEI, Polysciences) at a PEI:DNA ratio of 2.5:1. (Raymond C. et al. A simplified polyethylenimine-mediated transfection process for large-scale and high-throughput applications. Methods. 55(1):44-51 (2011)). Transfected cells were harvested after 5-6 days with the culture medium collected after centrifugation at 4000 rpm and clarified using a 0.45 μm filter. The sample was purified using the typical protein-A purification approach for antibodies. The purity of the samples was confirmed using UPLC SEC chromatography, SDS-PAGE and LCMS. FIG. 9 shows the SDS-PAGE of 5110 before and after SEC gel filtration and FIG. 10 shows the SEC gel filtration profile.

As shown in FIG. 9, V5110, the antibody—domain III fusion protein, is intact and expressed stably. The antibody-domain III fusion protein was prepared to a high degree of purity using standard methods for purification of antibodies, as shown in FIG. 10.

### Example 2

### Antibody Domain III Fusion Protein is Internalized by and Accumulates in Cells

The ability of the antibody domain III fusion protein to be internalized and to accumulate in cells was assessed and compared to the control trastuzumab antibody. The internalization assay was performed to determine the level of antibody uptake in JIMT-1 and SK-OV3 cancer cell lines. The experiment also assessed changes in cell surface binding, which could result from changes in the level of cell surface receptor. The experiment was based on the methods reported by Schmidt, M. et al., Kinetics of anti-carcinoembryonic antigen antibody internalization: effects of affinity, bivalency, and stability. Cancer Immunol Immunother (2008) 57:1879-1890, which involved directly labeling the antibody and antibody fusion proteins using the AlexaFluor® 488 Protein Labeling Kit (Invitrogen, cat. no. A10235), following the manufacturer's instructions. This method allows for labelling of the polypeptides with the AlexaFluor® label via lysine residues in the polypeptides. The mean fluorophore label level (DAR) on the antibody sample was in the range of 3.0 to 4.5.

In brief, 12-well plates were seeded with 1×10⁵ cells/well and incubated overnight at 37° C./5% CO₂. The following day, the labeled antibodies were added to the desired final concentration (e.g. 200 nM) in DMEM+10% FBS and the plates were incubated for 24 hours at 37° C./5% CO₂. In the dark, media was aspirated and wells were washed twice with 500 μL PBS. To harvest cells, cell dissociation solution (Sigma) was added (250 μL) at 37° C. Cells were pelleted and resuspended in 100 μL DMEM+10% FBS without or with anti-Alexa Fluor 488, rabbit IgG fraction (Molecular Probes, A11094, lot 1214711) at 50 μg/mL, and incubated on ice for 30 min. Prior to analysis, 300 μL of the cell suspension was filtered, and 4 μl propidium iodide was added. Samples were analyzed using the LSRII flow cytometer. A parallel cell binding experiment was performed at 4° C.

For each antibody or antibody fusion, 4 data points were recorded from the flow cytometer (Q₄, Q₃₇, U₄, U₃₇), corresponding to the MFI of cells bound by the labeled antibody at either 4° C. or 37° C., being either quenched or unquenched by the anti-Alexa Fluor 488 antibody. The initial receptor level (Sᵢ), final receptor level (Sₑ) and amount of antibody internalized (I) were calculated as follows:

quenching efficiency=QE=1−(Q₄/U₄)

initial surface receptor level (Surface 4° C.)=Sᵢ=U₄

final surface receptor level (Surface 37° C.)=S_{f}=(U₃₇−Q₃₇)/QE

antibody internalization/accumulation (Internal 37° C.)=I=U₃₇−S_{f}

Values were normalized by dividing Sᵢ, S_{f} and I by the DAR of the sample. The difference between base-line cell surface fluorescence at 4° C. and surface fluorescence measured after a 37° C. incubation period was an indication of receptor upregulation or down-regulation.

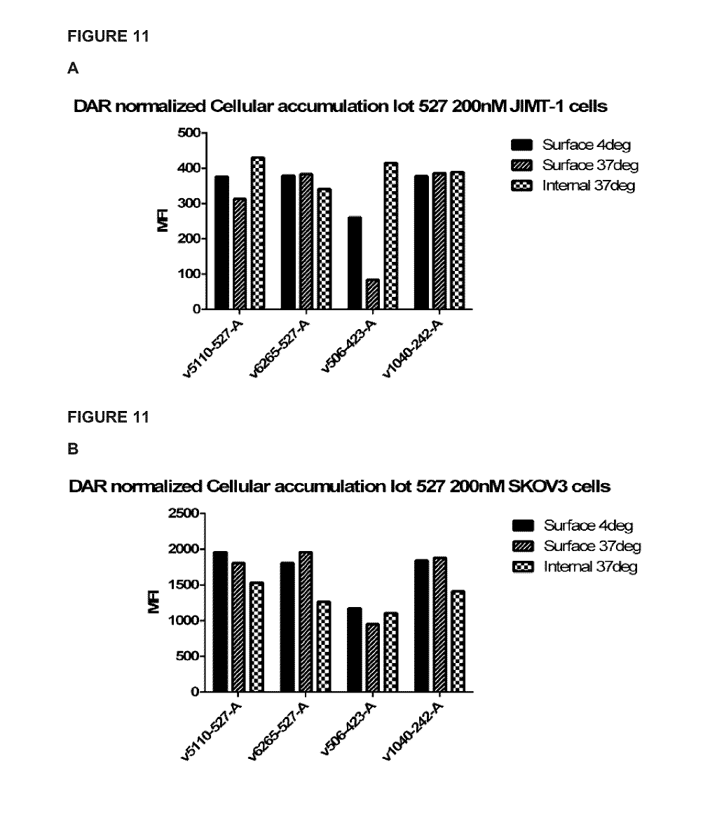

The results of this experiment are shown in FIG. 11 and in the following table.

TABLE A3
DAR-normalized MFI values:

| | JIMT-1 | | | SKOV3 | | |
|---|---|---|---|---|---|---|
| | Surface | Surface | Internal | Surface | Surface | Internal |
| | 4° C. | 37° C. | 37° C. | 4° C. | 37° C. | 37° C. |
| v1040 | 378 | 386 | 390 | 1842 | 1879 | 1409 |
| v506 | 261 | 84 | 415 | 1177 | 950 | 1107 |
| v5110 | 377 | 313 | 430 | 1960 | 1804 | 1533 |
| v6265 | 379 | 384 | 341 | 1804 | 1960 | 1264 |

The extent of cell surface and internal accumulation of antibody fused to domain III of antibody is comparable to the one armed antibody v1040 and greater than reference trastuzumab (v506).

### Example 3

### Conjugation of DM-1 to Antibodies

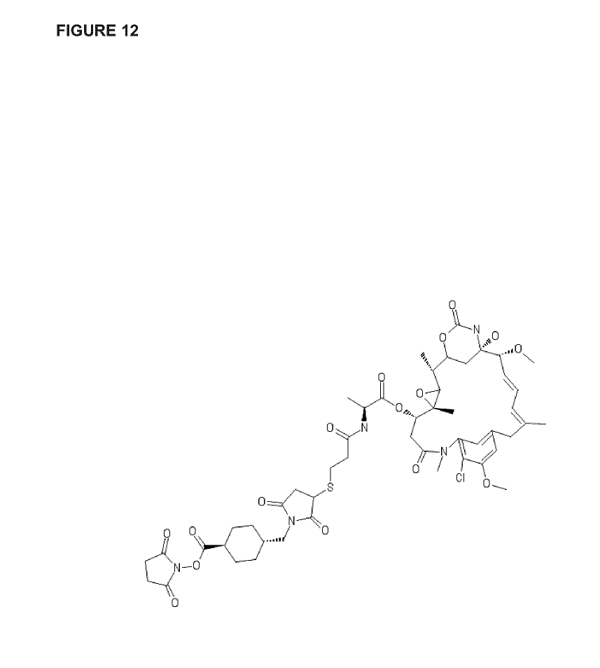

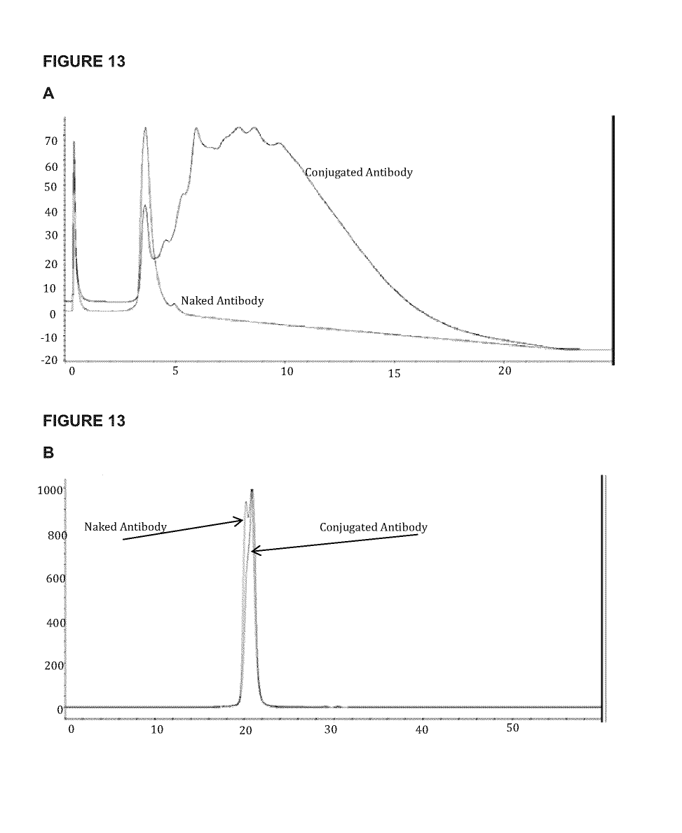

Antibody-drug conjugates (ADC) were generated as follows. The starting protein sample (antibody v1040, antibody DIII fusion v5110, or DIII v9992) was first buffer exchanged into 50 mM potassium phosphate pH 6.5, 50 mM NaCl and 2 mM EDTA using a PD-10 column, and adjusted to 10 mg/ml. A 10 mM solution of SMCC-DM1 (greater than 95% pure) (structure provided in FIG. 12) dissolved in dimethylacetamide (DMA) was then added to 7.0 molar equivalents of the protein sample. Alternate molar ratios were also employed to achieve different drug/protein conjugation ratios. DMA was further added to a final concentration of 10% v/v and the sample was mixed briefly. The reaction solution was incubated at 25° C. overnight with mixing. The reaction was monitored by determining the proportion of unconjugated protein sample by (hydrophobic interaction chromatography-high performance liquid chromatography) HIC-HPLC, and SMCC-DM1 was added in small increments until the amount of unconjugated sample was less than 5%. The product was then buffer exchanged into 20 mM sodium succinate pH 5.0 using a PD-10 column, and the protein concentration and drug-to-antibody ratio (DAR) were determined based on the absorbance at 252 and 280 nm. The buffer was adjusted to a final composition of 20 mM sodium succinate, 6% w/v trehalose and 0.02% w/v polysorbate 20, pH 5.0. High performance liquid chromatography-size exclusion chromatography (HPLC-SEC) was performed to identify any high molecular weight aggregate (FIG. 13A and FIG. 13B), which was purified out by SEC if it constituted more than 5% of the total protein content.

Table A4 provides the Drug/Protein Ratio upon conjugation of v5110 and v1040 at different molar ratios as determined using UV and LC-MS techniques. It is clear that under comparable conditions v5110 can achieve higher drug to protein ratio relative to v1040.

TABLE A4
Drug/Protein Ratio upon conjugation of v5110 and
v1040 at different molar ratios

| | Reaction Ratio | UV252 | |
|---|---|---|---|
| | (Drug:Protein, | and | |
| Variant | Molar equivalents) | UV280 | LC-MS |
| v5110 | 6.5:1 | 3.2 | 2.5 |
| v5110 | 7:1 | 3.7 | 2.6 |
| v5110 | 9:1 | 4.5 | 3.4 |
| v5110 | 11:1 | 5.4 | 4.2 |
| v1040 | 7:1 | 2.5 | 1.9 |
| v1040 | 8:1 | 3.3 | 3.3 |

V9992 was conjugated at three different molar equivalents (4:1, 6:1 and 8:1). The drug to protein ratio in the conjugated product evaluated by LC-MS indicated that the mean protein to drug ratio was 5.1, 5.5 and 5.4 respectively at the three reaction conditions with about 1% of the protein remaining unconjugated indicating a favorable conjugation profile. The drug to protein ratio is noticeably higher compared to typical antibody, especially under milder reaction conditions that has employed lower toxin to protein molar equivalents. Antibodies such as Trastuzumab at a 7.5:1 drug:protein reaction ratio yields a conjugated product with an average drug to protein ratio of about 3.5. T his indicates that v9992 (Domain III of human serum albumin) is better able to conjugate to toxin.

The ability of the ADCs to bind to the target antigen HER2 was assessed using SPR (surface plasmon resonance) binding. Briefly, binding by SPR was measured using the Bio-Rad ProteOn XPR36 instrument. Runs were carried out using 1×PBS running buffer at a temperature of 25 C. The target surface is generated using a GLC sensorchip via amine coupling kit (EDC/sNHS) activation followed by reaction with the purified antigen in 10 mM NaOAc at pH 4.5 until approximately 1000 RU signal is observed indicative of antigen immobilization. Remaining active groups are quenched by injection of enthanolamine. A 3-fold dilution series of the ADC with a blank buffer control is simultaneously injected at 50 mL/min for 120 s with a 20-minute dissociation, resulting in a set of binding sensograms with a buffer reference for each. A pulse of 0.85% phosphoric acid regenerates the antigen surface on the SPR chip.

The conjugation of the samples did not affect binding significantly. The following table summarizes the SPR data.

TABLE A5

| | KD | KD | | |
|---|---|---|---|---|
| | (Ave) | (STDEV) | STDEV/AVE | n |
| v5110 | 6.0E−10 | 8.4E−11 | 14% | 4 |
| v10135 (Conjugated v5110) | 7.8E−10 | 1.4E−10 | 18% | 4 |
| v1040 | 7.0E−10 | 9.0E−11 | 13% | 5 |
| v6247 (Conjugated v1040) | 8.0E−10 | 7.9E−11 | 10% | 4 |

These results confirm that domain III does not interfere with antigen binding.

### Example 4

### LC-MS Analysis of Antibody-Protein Drug Conjugates to Determine Regional DAR

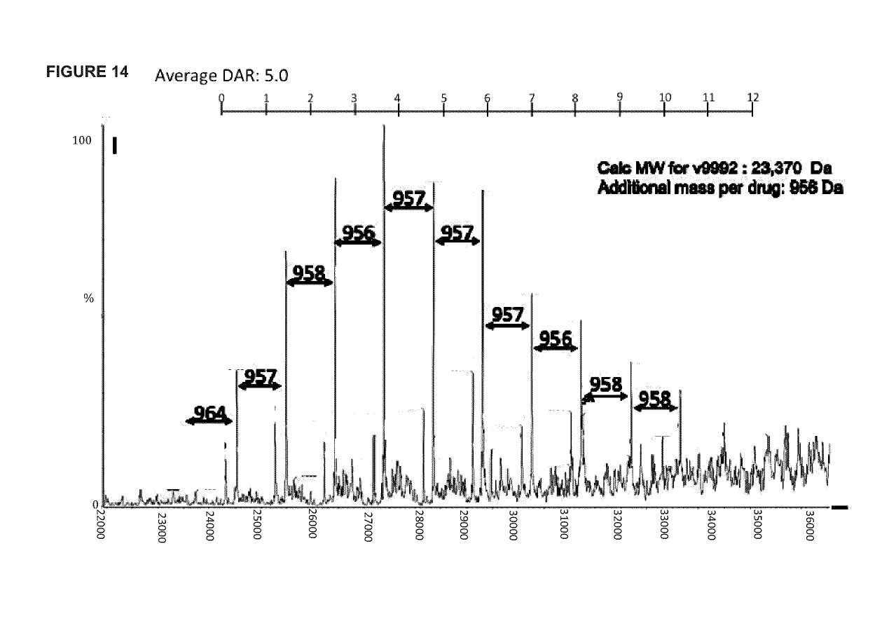

The regional DAR of the ADCs was assessed as described herein. The conjugated antibody and antibody-DIII fusion proteins were first deglycosylated using PNGase F treatment. The deglycosylated sample was incubated with 1 U IdeS (from Genovis) per ug of antibody in 150 mM NaCl, 50 mM sodium phosphate pH 6.6 for 30 minutes at 37° C. 0.1 M Tris-HCL pH 8 was added at 1:1 ratio. The sample was reduced with 200 mM DTT added at 1:10 ratio and heated at 56° C. for 30 minutes. 2.5 μg of the treated antibody sample in 10 μL 0.1% formic acid was analyzed on Agilent-PorosR2-Orbitrap LC-MS instrument at a Cone voltage of 10 V and FT resolution of 15000. A gradient of 10-75% of the solvent mixture (75% acetonitrile and 25% THF) was applied over 12 minutes. The relative peak intensities of deconvoluted mass spectra were employed to estimate the amount of drug conjugation to each fragment of the untreated parent antibody (FIG. 14).

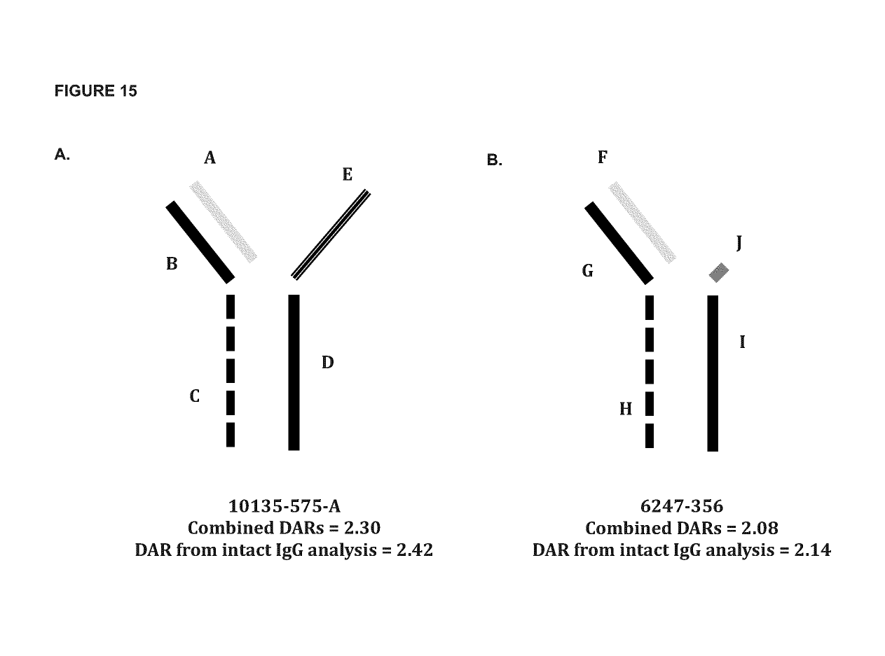

The results indicate that there was a reduction in the drug conjugation ratio for the Fc chains in the molecule comprising of the HSA-DIII (FIG. 15). Notably, about 51% of the net conjugation in the control v1040 antibody occurred on the Fc and in contrast the conjugation of Fc was down to about 29%, in the case of v5110. The fused HSA DIII component of v5110 had the maximum conjugation among all the domains in this molecule.

### Example 5

### Growth Inhibition

Growth inhibition assays were performed to determine the ability of the exemplary ADC's to inhibit the growth of cancer cell lines. The experiments were carried out as follows. Each well of a 96-well plate was seeded with 5000 JIMT-1 cells. Antibodies were added to final concentrations of up to 300 nM. The experiment was performed in triplicate. The final assay volume of the growth medium was 110 μL, and the 96-well plate was incubated at 37° C. for 5 days. The biomass within each well was then detected by measuring total protein with Sulforhodamine B following the manufacturers' instructions. The absorbance was read by a plate reader and the percentage of cell growth relative to the untreated control was calculated by:

% cell growth=100%×(RLUₛₐₘₚₗₑ)/(RLU_{untreated})

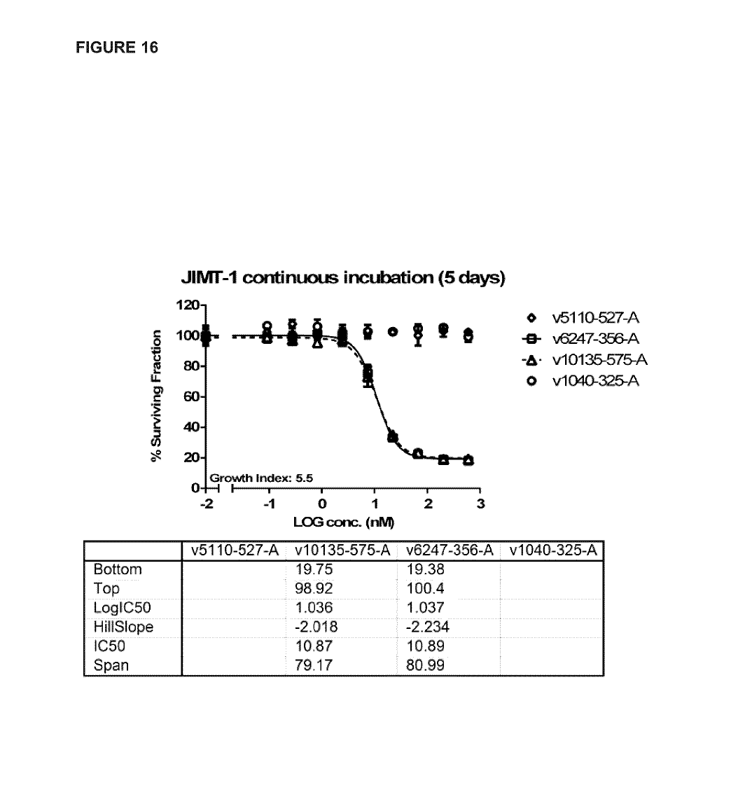

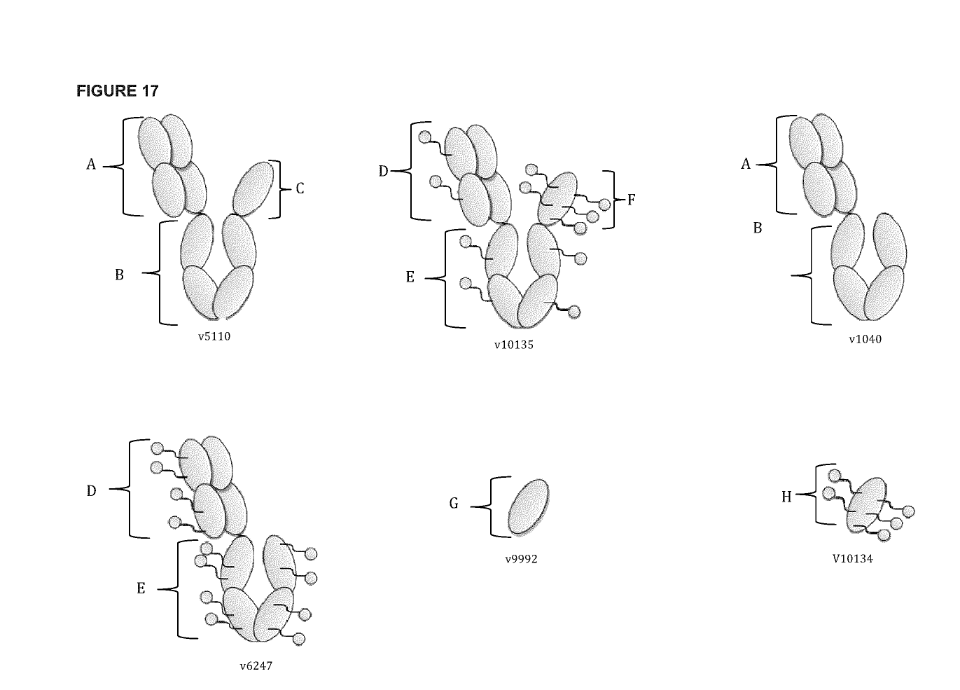

FIG. 16 shows the inhibition of JIMT-1 growth by conjugated v1040 (v6247) and v5110 (v10135) antibodies. The y-axis corresponds to the % growth relative to the untreated control. The x-axis corresponds to antibody concentration in nM. With a comparable DAR of about 2.3, both v6247 and v10135 show comparable level of growth inhibition and cell killing.

### Example 6

### Differential Scanning Calorimetry

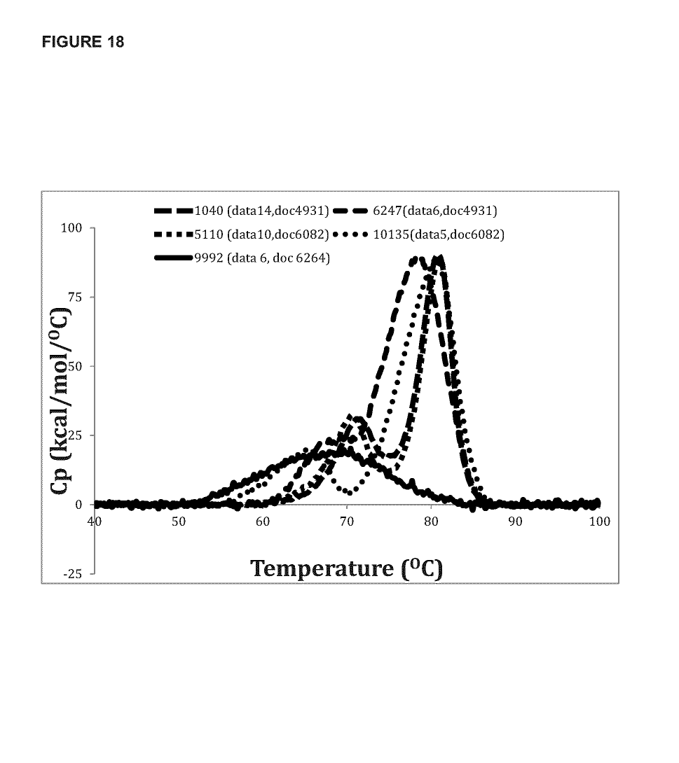

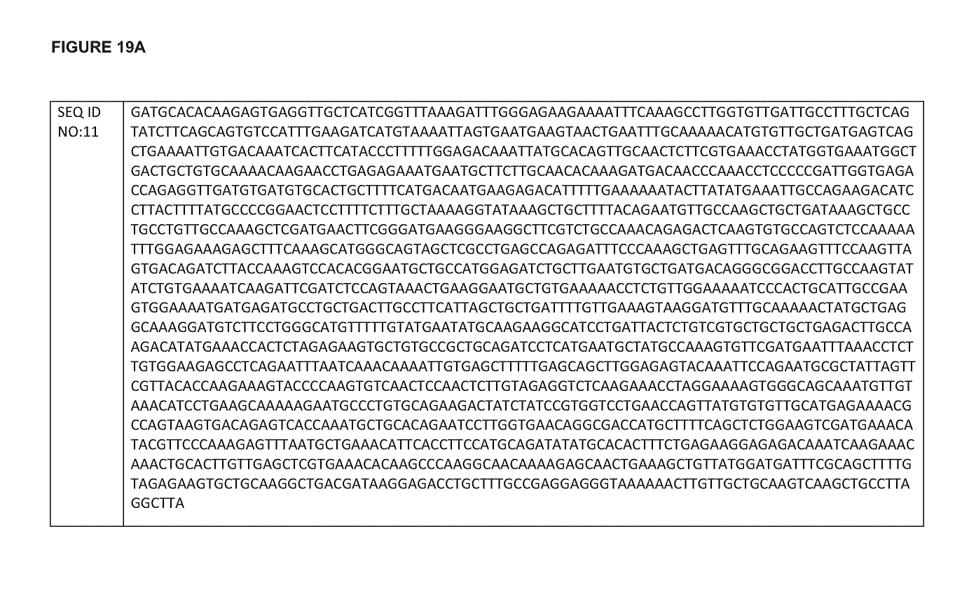

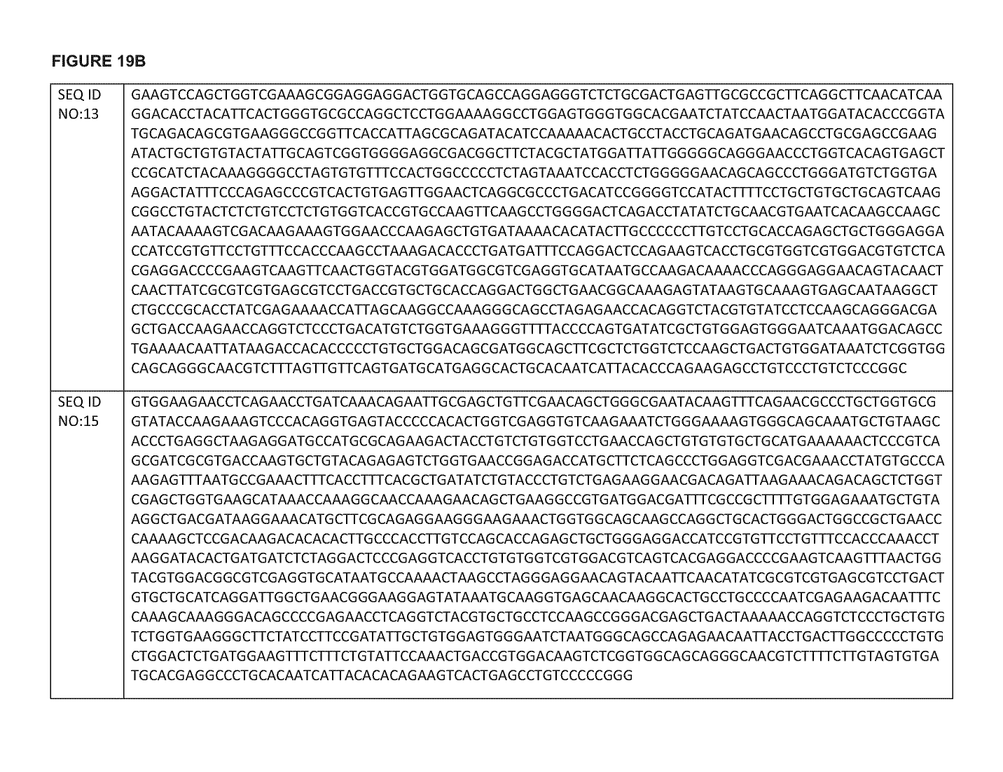

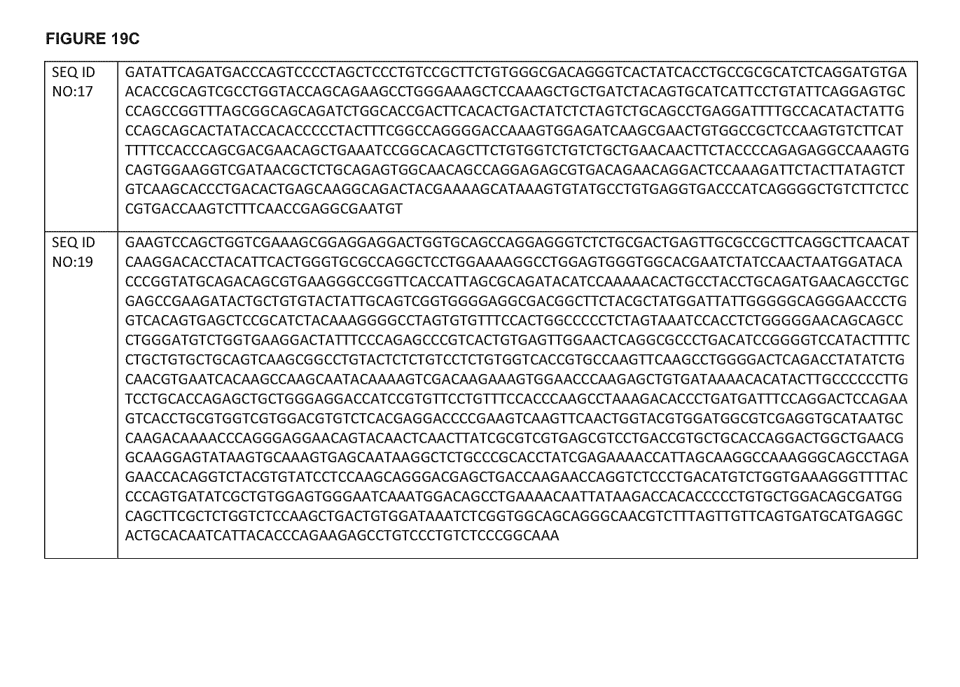

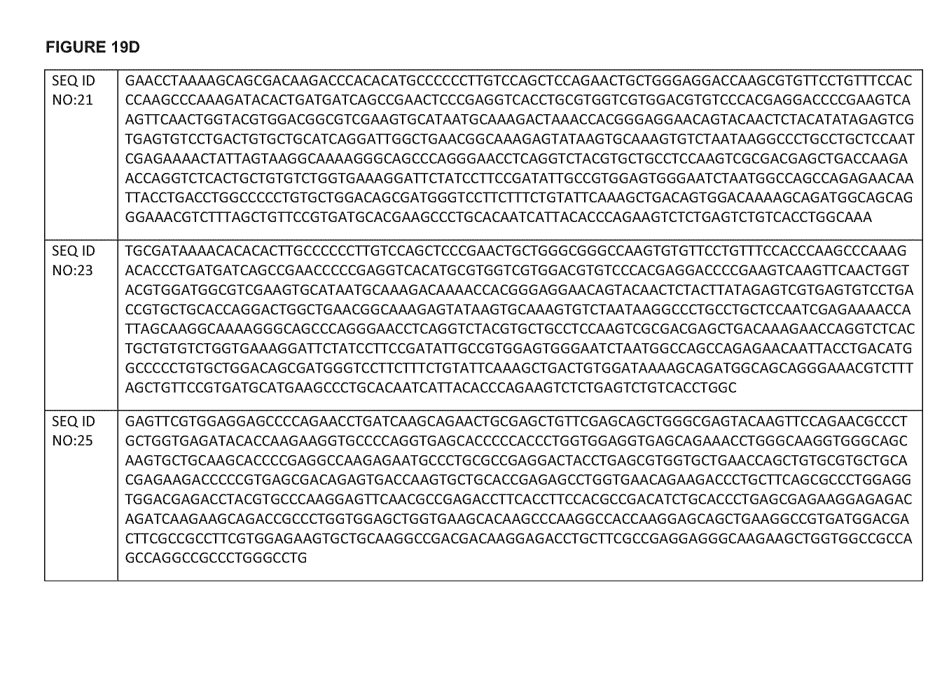

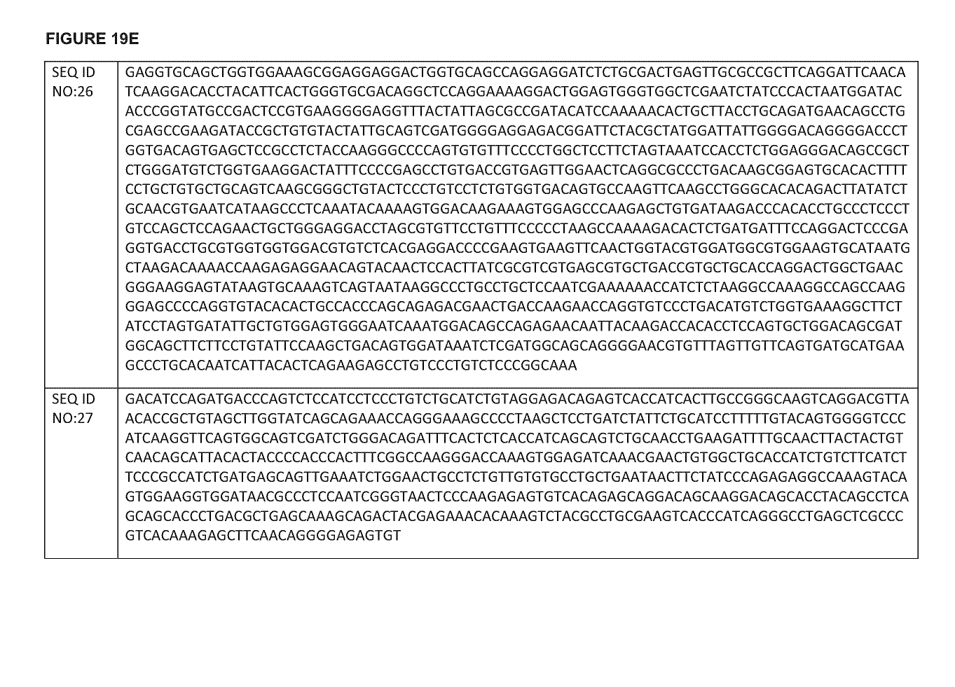

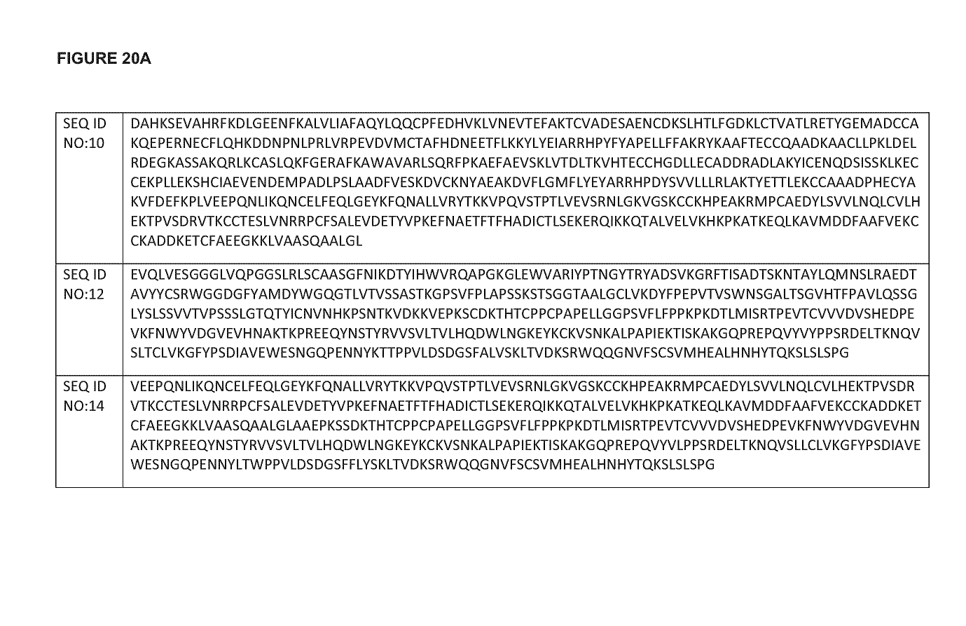

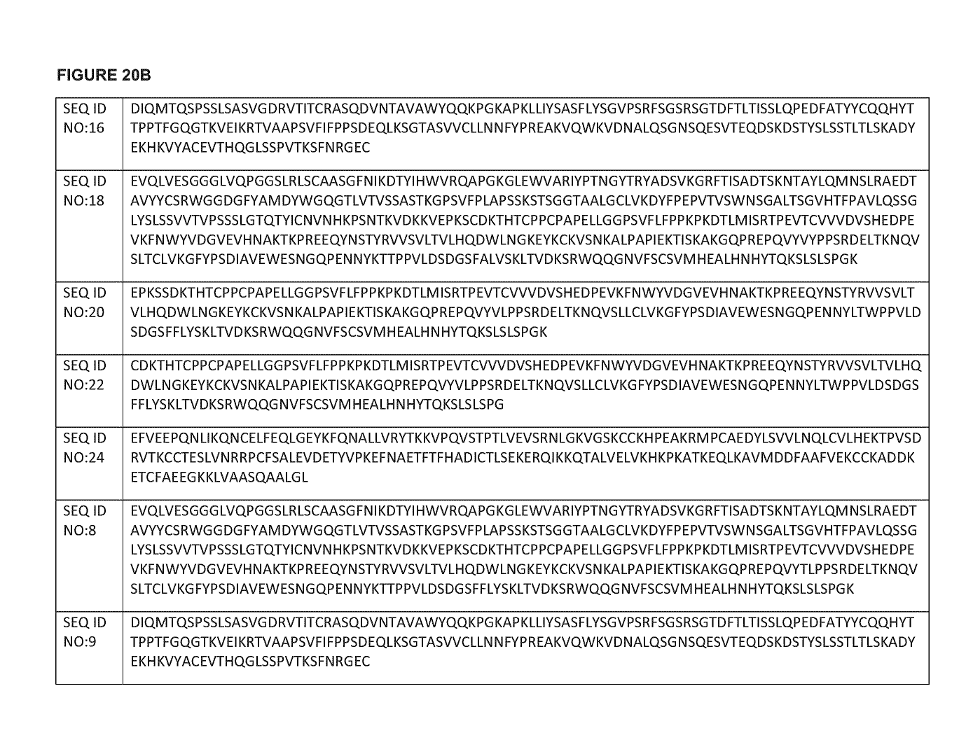

The thermal stability of the constructs was determined using differential scanning calorimetry as follows. Each antibody construct was diluted to ˜0.2 mg/mL in PBS, and a total of 400 μL was used for DSC analysis with a VP-Capillary DSC (GE Healthcare). At the start of each DSC run, five buffer blank injections were performed to stabilize the baseline, and a buffer injection was placed before each construct injection for referencing. Each sample was scanned from 20 to 100° C. at a 60° C./hr rate, with low feedback, 8 sec filter, 5 min preTstat, and 70 psi nitrogen pressure. The resulting thermograms were referenced and analyzed using Origin 7 software. The thermograms for variants 1040, 6247, 5110 and 10135 were normalized so that the peak height under the CH3+Fab transition was comparable. The results are presented in FIG. 18 and summarized in Table A6.

The broad unfolding transition observed for the isolated domain 3 of albumin (v9992) is not observed in the fusion sample (v5110), suggesting that the domain is more stable when fused. Upon conjugation, the first Tₘ of v5110 (composed of the CH2 domain and D3 of albumin) dropped from 70.6 to 65.4° C., but the second Tₘ (composed of the Fab and CH3 domain) only changed from 81.2 to 80.8° C. In contrast, for v1040 the first Tₘ (composed of the CH2 domain) dropped from 71.1 to −68.5° C. and the second Tₘ (composed of the Fab and CH3 domain) dropped from 80.7 to 78.2° C. The smaller drop in Tₘ for the Fab+CH3 transition in the fusion protein v5110 suggests that the presence of domain 3 of albumin decreased the destabilization undergone by the Fab+CH3 upon conjugation.

TABLE A6

| Variant | Tm1 | Tm2 |
|---|---|---|
| 1040 | 71.1 | 80.7 |
| 6247 | 68.5 | 78.2 |
| | (broad) | |
| 5110 | 70.6 | 81.2 |
| 10135 | 65.4 | 80.8 |

### Example 7

### Preparation of a Modular Protein Drug Conjugate by Ligation of a Toxin-Conjugated Polypeptide Therapeutic Agent Conjugation Module to an Antibody

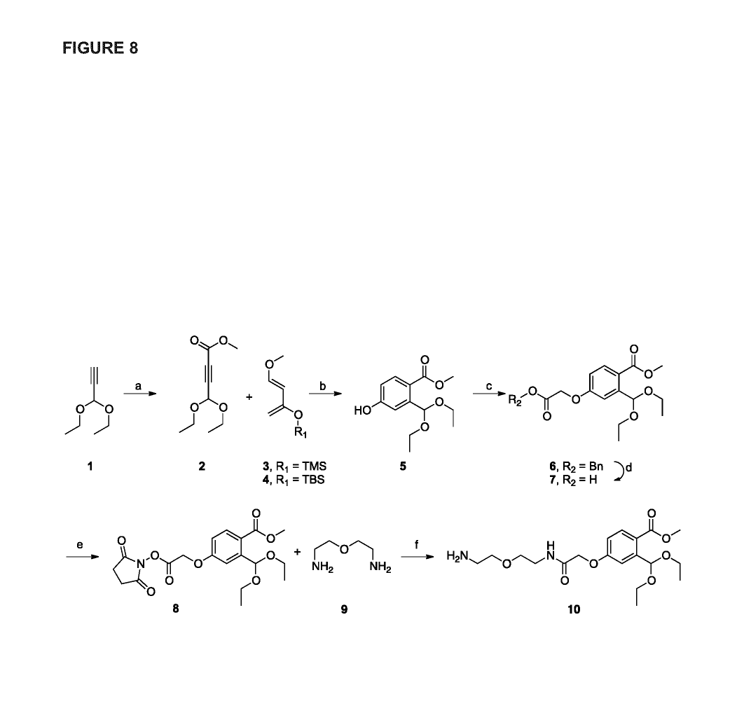

The following steps provide a reaction scheme to chemically link the conjugation module (protein B) that is selectively conjugated to the toxins to the targeting module (protein C) and presented schematically in FIGS. 5, 6, 7 and 8. Briefly, it comprises of the following steps:
(1) Bifunctional linker: Produce a bifunctional linker, compound 13, utilizing reaction scheme presented in FIG. 8 and steps a and b of FIG. 5. Compound 13 can be utilized for linking the toxin conjugation module and the targeting module.
(2) Functionalization of protein B: Functionalize the N-terminal residue of protein B (Domain III of human serum albumin) utilizing compound 13 in a transamination reaction in the presence of pyridoxyl-5′-phosphate (PLP). The functionalized protein B, depicted as compound 15, has an active functional group capable of capturing active cysteine residues. This reaction is presented in step c of FIG. 5.
(3) Toxin Conjugation: FIG. 6 presents the reaction of a maytansine derivative (compound 17) with compound 15 derived above. The number (n) of lysine residues in albumin domain III displace the NHS leaving group to yield a maytansine conjugated domain III of albumin depicted as compound 18.
(4) Conjugation—Targeting Module Conjugation: The reaction schematic in FIG. 7 presents the conjugation reaction of toxin loaded, functionalized domain III of albumin (protein B) (compound 18) with the N-terminal Cysteine residue of the antibody (protein C) (compound 19) to yield compound 20, the PDC of interest. The present invention provides, inter alia, novel antibody conjugates, and methods of using the conjugates. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

We claim:

1. A conjugate comprising:
(a) a polypeptide active agent conjugation module to which one or more active agents are covalently bound, the one or more active agents selected from therapeutic agents and diagnostic agents; and
(b) a polypeptide targeting module comprising two Fc polypeptides and one or more antigen-binding domains, said targeting module covalently bound to said active agent conjugation module.

2. The conjugate according to claim 1, wherein said one or more active agents are therapeutic agents.

3. The conjugate according to claim 2, wherein said one or more therapeutic agents are selected from Maytansinoids, Auristatins, Dolastatins, Calicheaminicin, Vinca Alkaloids, kinase inhibitors, alkylating agents and purine analogs.

4. (canceled)

5. The conjugate according to claim 1, wherein said targeting module is free from covalently bound active agent.

6. (canceled)

7. (canceled)

8. (canceled)

9. The conjugate according to claim 1, wherein said active agent is bound to a cysteine, lysine, or an analog or modified version thereof.

10. The conjugate according to claim 1, wherein said active agent conjugation module comprises albumin or an albumin fragment.

11. The conjugate according to claim 10, wherein said albumin fragment comprises amino acids 381 to 585 of wild type Albumin.

12. (canceled)

13. The conjugate according to claim 1, wherein a linker is interposed in said conjugate between said active agent and said active agent conjugation module; between said active agent conjugation module and said targeting module, or a combination thereof.

14. (canceled)

15. The conjugate according to claim 1, wherein said targeting module is a one-armed antibody.

16. The conjugate according to claim 15, wherein said one-armed antibody comprises a heterodimeric Fc domain fused to a single Fab arm.

17. (canceled)

18. A method of treating a disease in a subject in need of such treatment, said method comprising: administering to said subject therapeutically effective amount of a conjugate according to claim 2.

19. (canceled)

20. (canceled)

21. A conjugate comprising:
(a) a polypeptide therapeutic agent conjugation module to which one or more therapeutic agents are covalently bound, said therapeutic agent conjugation module comprising albumin or an albumin fragment; and
(b) a polypeptide targeting module comprising two Fc polypeptides and one or more antigen-binding domains, said targeting module covalently bound to said therapeutic agent conjugation module, wherein at least one of said antigen-binding domains specifically binds to HER2/neu.

22. The conjugate according to claim 21, wherein said targeting module is a one-armed antibody comprising antigen-binding domain sequences of trastuzumab.

23. The conjugate according to claim 21, wherein said two Fc polypeptides form a heterodimeric Fc domain.

24. The conjugate according to claim 21, wherein said one or more therapeutic agents comprise a maytansoid, and said therapeutic agent conjugation module is an albumin fragment.

25. The conjugate according to claim 21, wherein said therapeutic agent conjugation module is covalently bound to the C-terminus or the N-terminus of said targeting module.

26. (canceled)

27. The conjugate according to claim 21, wherein a linker is interposed in said conjugate between said therapeutic agent and said therapeutic agent conjugation module; between said therapeutic agent conjugation module and said targeting module, or a combination thereof.

28. The conjugate according to claim 21, wherein said conjugate comprises:
Heavy Chain A (SEQ. ID. NO.: 12), Heavy Chain B (SEQ. ID. NO.: 14), and light chain (SEQ. ID. NO.: 16).

29. (canceled)

30. (canceled)

31. The conjugate according to claim 1, wherein said active agent conjugation module is covalently bound to the C-terminus or the N-terminus of said targeting module.

32. The conjugate according to claim 1, wherein said active agent conjugation module comprises a lysine rich polypeptide.

33. The conjugate according to claim 1, wherein said active agent conjugation module comprises a fragment of albumin, a ubiquitin or a cell penetrating peptide.

34. The conjugate according to claim 1, wherein said targeting module comprises two Fc polypeptides and one antigen-binding domain.

35. The conjugate according to claim 34, wherein said antigen-binding domain is a Fab fragment or scFv.

36. The conjugate according to claim 1, wherein said two Fc polypeptides form a heterodimeric Fc domain.

37. The conjugate according to claim 36, wherein said heterodimeric Fc domain comprises one or more amino acid modifications in at least one CH3 sequence compared to a wild-type CH3 sequence.

38. The conjugate according to claim 37, wherein the heterodimeric Fc domain comprises the amino acid modifications:
(a) A:L231Y_F405A_Y407V, B:T366L_K392M_T394W;
(b) A:L231Y_F405A_Y407V, B:T366L_K392L_T394W;
(c) A:T350V_L351Y_F405A_Y407V, B:T350V_T366L_K392L_T394W;
(d) A:T350V_L351Y_F405A_Y407V, B:T350V_T366L_K392M_T394W, or
(e) A:T350V_L351Y_S400E_F405A_Y407V, B:T350V_T366L_N390R_K392M_T394W,
wherein the amino acid numbering is EU-numbering.

39. The conjugate according to claim 1, wherein the targeting module binds to a tumor-associated antigen.

40. The conjugate according to claim 39, wherein the tumor-associated antigen is an ErbB receptor associated antigen.

41. The conjugate according to claim 21, wherein said targeting module comprises two Fc polypeptides and one antigen-binding domain.

42. The conjugate according to claim 41, wherein said antigen-binding domain is a Fab fragment or scFv.

43. The conjugate according to claim 23, wherein said heterodimeric Fc domain comprises one or more amino acid modifications in at least one CH3 sequence compared to a wild-type CH3 sequence.

44. The conjugate according to claim 43, wherein the heterodimeric Fc domain comprises the amino acid modifications:
(a) A:L351Y_F405A_Y407V, B:T366L_K392M_T394W;
(b) A:L531Y_F405A_Y407V, B:T366L_K392L_T394W;
(c) A:T350V_L351Y_F405A_Y407V, B:T350V_T366L_K392L_T394W;
(d) A:T350V_L351Y_F405A_Y407V, B:T350V_T366L_K392M_T394W, or
(e) A:T350V_L351Y_S400E_F405A_Y407V, B:T350V_T366L_N390R_K392M_T394W,
wherein the amino acid numbering is EU-numbering.

45. A method of treating cancer in a subject in need of such treatment, said method comprising: administering to said subject a therapeutically effective amount of a conjugate according to claim 21.
